(19) United States
(12) Patent Application Publication
(10) Pub. No.: US 2025/0034185 A1
(43) Pub. Date: Jan. 30, 2025

(54) PYRIDOXAL-5-PHOSPHATE (P5P) ANALOGS

(71) Applicant: CanAm Bioresearch Inc., Winnipeg (CA)

(72) Inventors: Albert D. Friesen, Winnipeg (CA); Jean-d'amour K. Twibanire, Winnipeg (CA); Kevin P. Carlin, Winnipeg (CA); Matinder Kaur, Winnipeg (CA); Rafiq A. Taj, Winnipeg (CA); Syed Mohammed A. Hussani, Winnipeg (CA); Vaneet Chehal, Winnipeg (CA); Ramkrishna Reddy Vakiti, Winnipeg (CA); Bhavin Pipaliya, Winnipeg (CA); Cyril Cook, Winnipeg (CA); Qasim Khan, Winnipeg (CA); Farman Ullah, Winnipeg (CA)

(21) Appl. No.: 18/715,506

(22) PCT Filed: Dec. 15, 2022
(86) PCT No.: PCT/CA2022/051832

§ 371 Date: May 31, 2024

Related U.S. Application Data
(60) Provisional application No. 63/290,050, filed on Dec. 15, 2021.

(51) Int. Cl. C07F 9/58; A61K 31/675; C07F 9/6558

(52) U.S. Cl. CPC C07F 9/58; A61K 31/675; C07F 9/65583

(57) ABSTRACT

Disclosed are compound, its stereoisomer, salt, hydrate, solvate, isotope or crystalline form thereof, represented by Formula (I), with X, R¹, R², R³, R⁴ and R⁵ as disclosed herein. Also, disclosed is a process for their preparation, compositions containing such compounds, methods of medical treatment or prophylaxis of a disease by such compounds and uses thereof. The compounds of Formula (I) are pyridoxal-5-phosphate (P5P) analogs, and can act as prodrugs.

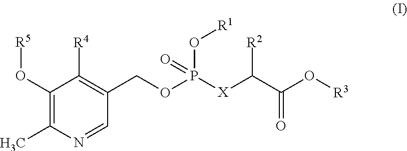

### FIELD

This specification relates to pyridoxal-5-phosphate (P5P) analogs, process for their preparation, compositions containing pyridoxal-5-phosphate (P5P) analogs, methods of medical treatment containing pyridoxal-5-phosphate (P5P) analogs and uses of the same.

### BACKGROUND

Vitamin B₆ is necessary for normal functioning of the human body. Deficiency in this vitamin leads to dysfunction and disease (Stach, et al. (2021) Vitamin B6 in Health and Disease. Nutrients, 13, 3229, incorporated herein by reference). Vitamin B₆ is not a single molecule, but rather a group of related molecules—vitamers. These molecules include pyridoxal, pyridoxamine, pyridoxine and their respective 5′-monophosphorylated forms. The human body cannot synthesize these molecules de novo and therefore some of the B₆ forms need to be obtained through dietary means. Once in the human physiological system, the various vitamers can be interconverted (FIG. 1; Stach, et al. (2021)).

Pyridoxal-5-phosphate (P5P) is a physiologically important form of vitamin B₆. It is in this form that vitamin B₆ is a co-factor in more than 150 biochemical reactions (Stach, et al. (2021)). Therefore, by manipulating the amount of P5P in the body, one can affect wellness and mitigate disease. However, due to the fact that this form is phosphorylated, and therefore charged at physiological pH, it cannot readily pass-through biological membranes (Alghamdi, et al. (2021) Phenotypic and molecular spectrum of pyridoxamine-5′-phosphate oxidase deficiency: A scoping review of 87 cases of pyridoxamine-5′-phosphate oxidase deficiency. Clin Genet 99, 99-110, incorporated herein by reference). As such, P5P has poor oral bioavailability (Vrolijk et al., (2020) Inter-individual differences in pharmacokinetics of vitamin B6: A possible explanation of different sensitivity to its neuropathic effects. PharmaNutrition 12, 1-7; incorporated herein by reference) with limited ability to be absorbed across the gastrointestinal membranes. To circumvent this issue, two approaches have been taken, wherein the first approach vitamin B₆ is given orally as pyridoxine which is better absorbed. The body then converts a portion of the pyridoxine to P5P over time (Vrolijk et al., 2020; Coburn et al., (1991) Response of vitamin B-6 content of muscle to changes in vitamin B-6 intake in men. Am J Clin Nutr 53, 1436-42; all incorporated herein by reference). Unfortunately, oral pyridoxine (unlike oral P5P) leads to increased plasma levels of pyridoxine which has shown evidence of neurotoxicity (Vrolijk et al., (2017) The vitamin B₆ paradox: Supplementation with high concentrations of pyridoxine leads to decreased vitamin B6 function. Toxicol In Vitro 44, 206-212; and Hadstein (2021) Vitamin B-6-Induced Neuropathy: Exploring the Mechanisms of Pyridoxine Toxicity. Adv Nutr 12, 1911-1929; all incorporated herein by reference). In the second approach, large amounts of P5P are given orally in order to achieve a therapeutic level in the biological system (Gibaud et al., et al. (2021) West Syndrome Is an Exceptional Presentation of Pyridoxine- and Pyridoxal Phosphate-Dependent Epilepsy: Data From a French Cohort and Review of the Literature. Front Pediatr 9, 621200; and Sudarsanam et al., (2014) Cirrhosis associated with pyridoxal 5′-phosphate treatment of pyridoxamine 5′-phosphate oxidase deficiency. JIMD Rep 17, 67-70; all incorporated herein by reference). Unfortunately, this can cause gastrointestinal issues and may contribute to hepatotoxicity (Sudarsanam et al., (2014); Alghamdi et al., (2021) Phenotypic and molecular spectrum of pyridoxamine-5′-phosphate oxidase deficiency: A scoping review of 87 cases of pyridoxamine-5′-phosphate oxidase deficiency. Clin Genet 99, 99-110; all incorporated herein by reference).

ProTide technology was developed by Chris McGuigan at Cardiff University in the early 1990's as a method to deliver phosphorylated nucleosides (nucleotides) into cells. This technology has mainly been used to deliver monophosphorylated antiviral agents in cells (Mehellou et al., (2018) The ProTide Prodrug Technology: From the Concept to the Clinic. J Med Chem 61, 2211-2226; Thornton et al., (2016) Nucleoside Phosphate and Phosphonate Prodrug Clinical Candidates. J Med Chem 59, 10400-10410; Markovic et al., (2020) Prodrugs for Improved Drug Delivery: Lessons Learned from Recently Developed and Marketed Products. Pharmaceutics 12, 1031; all incorporated herein by reference) and to improve the oral bioavailability of some of these same agents (such as, sofosbuvir). Schwarz et al. (Schwarz et al., (2020) A Phosphoramidate Strategy Enables Membrane Permeability of a Non-nucleotide Inhibitor of the Prolyl Isomerase Pin1. ACS Med. Chem. Lett. 11, 1704-1711, incorporated herein by reference) have disclosed membrane permeability of a non-nucleotide inhibitor of the prolyl isomerase Pint.

The basic premise of the ProTide technology is the use of various chemical groups to hide the charges that exist on a phosphate group at physiological pH. These groups are specialized, in that one group is an amino acid ester and the other group is chemically a good leaving group, usually an aryl group or the like. The resultant phosphoramidate created by the amino acid ester group provides stability to the molecule while the ester portion is the substrate for enzymatic esterase action. It is cleavage of this ester that starts the sequence of chemical rearrangements that ultimately releases the drug of interest (FIG. 2; taken from Schwarz et al., (2020) A Phosphoramidate Strategy Enables Membrane Permeability of a Non-nucleotide Inhibitor of the Prolyl Isomerase Pin1. ACS Med. Chem. Lett. 11, 1704-1711, incorporated herein by reference). This release of drug can occur in the plasma or intracellularly.

There is a need in the art to develop analogs of vitamin B₆ (P5P) with improved bioavailability that can more readily enter the biological system, as such analogs can be useful for treating diseases and maintaining health. Such analogs could permit discontinued use of the potentially neurotoxic pyridoxine form and circumvent the use of large oral doses of P5P. In addition, there is a need in the art for a process for preparation of such P5P analogs. Further, there is a need in the art for compositions containing such P5P analogs. Moreover, there is a need in the art for use of such P5P analogs for treatment of a disease, or a method of medical treatment of a disease by administration of such P5P analogs.

### SUMMARY OF THE SPECIFICATION

The specification relates to prodrugs of pyridoxal-5-phosphate (P5P) and prodrugs of analogs of P5P. In addition, the specification relates to compounds of formula (I) including its pharmaceutically-acceptable salts, hydrates, solvates, stereoisomers, and pharmaceutical compositions of these compounds which can be useful for preventive and therapeutic use in human and in veterinary medicine. Another aspect is an isotopically labeled compound of any of the formulae delineated herein. Such compounds have one or more isotope atoms which may or may not be radioactive (e.g., ³H, ²H, ¹⁴C, ¹³C, ¹⁸F, ³²P) introduced into the compound. Such compounds can be useful for drug metabolism studies and diagnostics, as well as therapeutic applications.

In a first aspect, the specification relates to a compound, its stereoisomer, salt, hydrate, solvate, isotope or crystalline form thereof, represented by formula I:

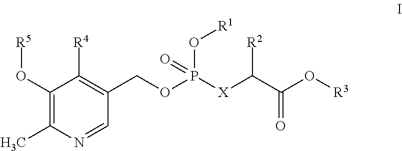

wherein X, R¹, R², R³, R⁴ and R⁵ are as described herein.

In a second aspect, the specification relates to a process for preparation of a compound, its stereoisomer, salt, hydrate, solvate, isotope or crystalline form thereof, of Formula I:

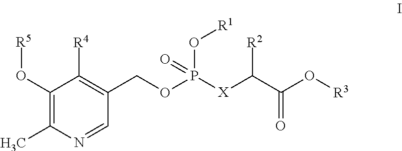

the process comprising the step of reacting the compound of Formula II with a compound of Formula III, in the presence of a base, to form the compound of Formula I

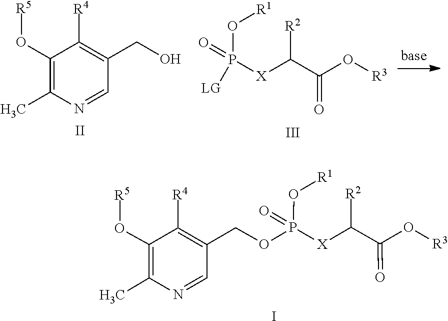

wherein X, LG, R¹, R², R³, R⁴ and R⁵ are as described herein.

In a third aspect, the specification relates to a composition comprising:
a carrier, diluent or excipient, and
a compound, its stereoisomer, salt, hydrate, solvate, isotope or crystalline form thereof, represented by formula I:

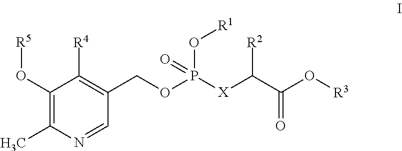

wherein X, R¹, R², R³, R⁴ and R⁵ are as described herein.

In a fourth aspect, the specification relates to a use of a compound, its stereoisomer, salt, hydrate, solvate, isotope or crystalline form thereof, represented by formula I, for treatment or prophylaxis of a disease:

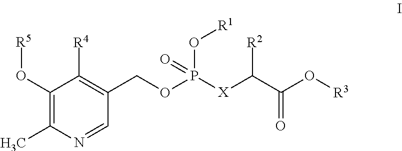

wherein X, R¹, R², R³, R⁴ and R⁵ are as described herein.

In a fifth aspect, the specification relates to a method of medical treatment or prophylaxis of a disease, comprising administering, to a subject in need thereof, a compound, its stereoisomer, salt, hydrate, solvate, isotope or crystalline form thereof, represented by formula I:

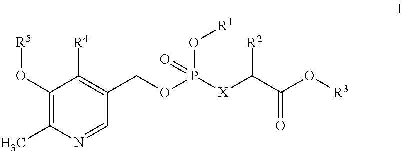

wherein X, R¹, R², R³, R⁴ and R⁵ are as described herein.

### BRIEF DESCRIPTION OF THE DRAWINGS

Reference will now be made, by way of example, to the accompanying drawings which show example embodiments of the present application, and in which:

FIG. 1 shows the various vitamers that can be interconverted once in the human physiological system;

FIG. 2 shows a schematic representation of the ability of the phosphoramidate to enter the cytosol;

FIGS. 3 a-h show the results of the maximum tolerability of the test article in rats under 4 different doses under one single administration via PO route;

FIGS. 4 a & b show the dose-dependent increases in plasma P5P levels upon administration of compound of formula 16i, confirm effectiveness of the prodrug;

FIGS. 5 a & b show the results of the linearity of pharmacokinetics of the compound of formula 16 under multiple doses;

FIGS. 6 a-d show the difference in pharmacokinetic profile of prodrug 16 (given IV) compared to PLP given IV;

FIGS. 7 a-d show the improved ability of prodrug 16 (given PO) to increase plasma PLP compared to of PLP itself;

FIGS. 8 a-b show individual rat data demonstrating the difference in PLP levels when PLP is orally administered as PLP compared to using a protide prodrug. Average data is also shown;

FIGS. 9 a-d compares the effect of a different prodrug 16v (a & b) to 16 (c & d) at 1 mg/kg administered intravenously;

FIGS. 10 a-d compares the effect of a different prodrug 16v (a and b) to 16 (c and d) at 10 mg/kg administered orally;

FIGS. 11 a-d show the results of the average plasma concentration of PL, P5P and the compound not encompassed by Formula I at 1 mg/kg and 10 mg/kg;

FIGS. 12 a-c show the results of the average plasma concentration of PL, P5P and the compound of Formula 16vi at 1 mg/kg and 10 mg/kg;

FIGS. 13 a-h show the results of the average plasma concentration of PL, P5P and the compounds of Formula 16i (single isomer; a & b), 16ii (single isomer; c & d), and 16 (racemate, two samples; e & h);

FIGS. 14 a-h show the results of the average plasma concentration of PL, P5P and the compounds S-isomer (16i) and the R-isomer (16ii) at the phosphorus center; results of both IV and PO administration shown;

FIGS. 15 a-f show the results of the average plasma concentration of PL, P5P and the compounds S-isomer (16i) and the R-isomer (16ii) and 16 (racemate);

FIGS. 16 a-d show the results of the average plasma concentration of PL, P5P and PLP prodrug 16iii (racemate with D amino acid) at 1 mg/kg (IV administered, a & b) and at 10 mg/kg (PO administered—c & d); and

FIGS. 17 a-d show the results of the average plasma concentration of PL, P5P and PLP prodrug 16iii (racemate with D amino acid) in comparison to 16 (racemate) at 10 mg/kg via PO administration.

Similar reference numerals may have been used in different figures to denote similar components.

### DESCRIPTION

The specification includes headings and sub-headings solely to assist with review and understanding of the specification, and disclosure under any heading or sub-heading is not limited to the particular section of the specification, but rather should be considered in light of the entire disclosure of the specification.

### Compounds

In an aspect, the specification relates to a compound, its stereoisomer, salt, hydrate, solvate, isotope or crystalline form thereof, represented by formula I:

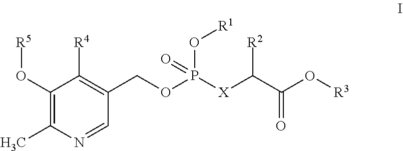

Wherein
X is NH or N;
R¹ is H or a C₁₋₁₅ substituent having one or more heteroatoms;
R² is H or a C₁₋₁₅ substituent having one or more heteroatoms, or when X is N, R² and N form a cyclic structure having C₂₋₁₅ atoms having one or more heteroatoms;
R³ is H or a C₁₋₁₅ substituent having one or more heteroatoms;
R⁴ is

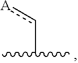

wherein [symbol] is a single or a double bond, and wherein
when [symbol] is a single bond,
A is NH₂, NH-PG_{N},

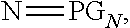

OH, O-PG_{O1}, where PG_{N} is a protecting group bonded to a nitrogen, and PG_{O1} is a first protecting group bonded to an oxygen;
when [symbol] is a double bond
A is O; and
R⁵ is H or PG_{O2}, wherein PG_{O2} is a second protecting group bonded to oxygen, or PG_{O1} and PG_{O2} together form a diol protecting group.

The term ‘stereoisomer’ as used herein is not particularly limited and should be known to a person of skill in the art. Stereoisomers, stereoisomerism, or spatial isomerism, is a form of isomerism in which molecules have the same molecular formula and sequence of bonded atoms (constitution), but differ in the three-dimensional orientations of their atoms in space. Enantiomers and diastereomers are two types of stereoisomers. Enantiomers, also known as optical isomers, are two stereoisomers that are related to each other by a reflection: they are mirror images of each other that are non-superposable. Diastereomers are stereoisomers not related through a reflection operation. They are not mirror images of each other. In stereochemistry, a stereocenter of a molecule is an atom, axis or plane that is the focus of stereoisomerism; that is, when having at least three different groups bound to the stereocenter, interchanging any two different groups creates a new stereoisomer.

The compounds disclosed herein have a number of stereocenters, including the P (phosphorous), which can be chiral center, and that it has a corresponding Cahn-Ingold-Prelog designation of “R” or “S” which have their accepted plain meanings. It is contemplated that compounds of the formula I can be racemic, stereochemically pure or have one stereoisomer present predominantly because of the chirality at phosphorous. Applicants contemplate use of the racemate and/or the resolved enantiomers.

The present application is not particularly limited, and includes all possible stereoisomers, and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art (See, for example, “Stereochemistry of Organic Compounds” by E. L. Eliel, S. H. Wilen, and L. N. Mander (Wiley30 Interscience, 1994), incorporated herein by reference).

The term ‘salt’ as disclosed herein is not particularly limited and should be known to a person of skill in the art, or can be determined. The salt of the compound of Formula I formed can depend upon the application, and includes pharmaceutically acceptable salts. A “pharmaceutically acceptable salt” form of the compound of Formula I may also initially confer a desirable pharmacokinetic property on the compound which were absent in the non-salt form, and may even positively affect the pharmacodynamics of the compound of Formula I with respect to its therapeutic activity in the body. The phrase “pharmaceutically acceptable salt” of a compound as used herein means a salt that is pharmaceutically acceptable and that possesses the desired pharmacological activity of the parent compound. Such salts include, for example and without limitation: (1) acid addition salts, formed with inorganic acids such as hydrochloric acid, hydrobromic acid, sulfuric acid, nitric acid, phosphoric acid, and the like; or formed with organic acids such as glycolic acid, pyruvic acid, lactic acid, malonic acid, malic acid, maleic acid, fumaric acid, tartaric acid, citric acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulfuric acid, gluconic acid, glutamic acid, salicylic acid, muconic acid, and the like or (2) basic addition salts formed with the conjugate bases of any of the inorganic acids listed above, wherein the conjugate bases comprise a cationic component selected from among Na⁺, K⁺, Mg²⁺, Ca²⁺, and NH_{g}R′_{4-g}⁺, in which R′ is a C₁₋₃ alkyl and g is a number selected from among 0, 1, 2, 3, or 4. It should be understood that all references to pharmaceutically acceptable salts include solvent addition forms (solvates) or crystal forms (polymorphs) as described herein, of the same acid addition salt.

The term ‘hydrate’ as used herein is not particularly limited and should be known to a person of skill in the art. In general, a hydrate is a substance that contains water or its constituent elements, and can be formed by the addition of water. In addition, the compound of Formula I can form crystals that can incorporate water into the crystalline structure without chemical alteration of the compound (water of crystallization). It should be known that hydrate formation is common for many active ingredients. Many manufacturing processes provide an opportunity for hydrates to form and the state of hydration can be changed with environmental humidity and time. The state of hydration of an active pharmaceutical ingredient can significantly affect the solubility and dissolution rate and therefore its bioavailability.

The term ‘solvate’ as used herein is not particularly limited and should be known to a person of skill in the art. A solvate is similar to a hydrate, however, rather than water, a solvent is present. In broad terms, a solvate is an aggregate that consists of a solute ion or molecule with one or more solvent molecules. The specification encompasses solvates and hydrates of the compounds disclosed herein.

The term ‘isotope’ as used herein is not particularly limited and should be known to a person of skill in the art. Isotopes are two or more types of atoms that have the same atomic number (number of protons in their nuclei) and position in the periodic table (and hence belong to the same chemical element), and that differ in nucleon numbers (mass numbers) due to different numbers of neutrons in their nuclei. The specification encompasses compounds disclosed herein having a varying number and type of isotopes. For example and without limitation, the compound of Formula I can have 1, 2, 3, 4, 5, 6, 7, 8, 9, 10, 11, 12, 13, 14, 15 or more isotopes present. In addition, for example and without limitation, the compound of Formula I includes compounds where ¹H is replaced with ²D or ³T, ¹²C is replaced with ¹³C or ¹⁴C, and/or ³¹P is replaced with ³²P or ³³P. In preparing isotopes, appropriate starting material or intermediate can be used that is isotopically enriched and having the desired isotope.

The term ‘crystalline form’ as used herein is not particularly limited and should be known to a person of skill in the art. A crystal or crystalline form is a solid material whose constituents (such as atoms, molecules, or ions) are arranged in a highly ordered microscopic structure, forming a crystal lattice that extends in all directions.

The term ‘C₁₋₁₅ substituent’ and the like is not particularly limited and should be known to a person of skill in the art. A C₁₋₁₅ substituent relates to an organic moiety having one to fifteen carbon atoms, and can include, for example and without limitation, alkyl, alkenyl, alkynyl, cycloalkyl, aryl, heteroaryl, cycloalkyl alkyl, cycloheteroalkyl, alkyl aryl, alkyl heteroaryl and like, which can have one or more heteroatoms. In one embodiment, for example and without limitation, the number of carbon atoms present can be 1, 2, 3, 4, 5, 6, 7, 8, 9, 10, 11, 12, 13, 14 or 15, including a range based on any combination of the number of carbon atoms noted herein.

In one embodiment, for example and without limitation, R¹ in the compound of Formula I is H or a C₁₋₁₅ substituent having one or more heteroatoms. In a second embodiment, for example and without limitation, R¹ in the compound of Formula I is an organic moiety having 1, 2, 3, 4, 5, 6, 7, 8, 9, 10, 11, 12, 13, 14 or 15 carbon atoms, including a range based on any combination of the number of carbon atoms noted herein (for example and without limitation, C₁₋₁₅, C₁₋₁₀, C₁₋₇, C₃₋₁₅, C₃₋₁₀, C₃₋₇, C₅₋₁₅, C₅₋₁₀, C₅₋₇, and the like), which have one or more heteroatoms. In a third embodiment, for example and without limitation, R¹ in the compound of Formula I is —C₆H₅, —C₆H₄Cl, —C₆H₃BrF, —C₆H₃Cl₂, —C₆D₅, —C₇H₇, —C₇H₄OF₃, —C₇H₇O, —C₉H₉, —C₁₀H₁₁ or —C₁₀H₇.

In one embodiment, for example and without limitation, R² in the compound of Formula I is H or a C₁₋₁₅ substituent having one or more heteroatoms, or when X is N, R² and N form a cyclic structure having C₃₋₁₅ atoms having one or more heteroatoms. In a second embodiment, for example and without limitation, when R² and N do not form a cyclic structure, R² in the compound of Formula I is an organic moiety having 1, 2, 3, 4, 5, 6, 7, 8, 9, 10, 11, 12, 13, 14 or 15 carbon atoms, including a range based on any combination of the number of carbon atoms noted herein (for example and without limitation, C₁₋₁₅, C₁₋₁₀, C₁₋₇, C₃₋₁₅, C₃₋₁₀, C₃₋₇, C₅₋₁₅, C₅₋₁₀, C₅₋₇, and the like), which have one or more heteroatoms. In a third embodiment, for example and without limitation, when R² and N form a cyclic structure, R² in the compound of Formula I is an organic moiety having 3, 4, 5, 6, 7, 8, 9, 10, 11, 12, 13, 14 or 15 carbon atoms, including a range based on any combination of the number of carbon atoms noted herein (for example and without limitation, C₃₋₁₅, C₃₋₁₀, C₃₋₇, C₃₋₁₅, C₅₋₁₀, C₅₋₇, C₆₋₁₅, C₆₋₁₀, C₆₋₇, and the like), which have one or more heteroatoms. In a fourth embodiment, for example and without limitation, R² in the compound of Formula I is —H, -D, —CH₃, —C₂H₂F₃, —CH₂CH₂CH₂—, —C₄H₉, —C₅H₉, —C₆H₅, —C₇H₇, —C₇H₇O, —C₁₄H₁₂O or —C₁₄H₁₆NO₂.

In one embodiment, for example and without limitation, R³ in the compound of Formula I is H or a C₁₋₁₅ substituent having one or more heteroatoms. In a second embodiment, for example and without limitation, R³ in the compound of Formula I is an organic moiety having 1, 2, 3, 4, 5, 6, 7, 8, 9, 10, 11, 12, 13, 14 or 15 carbon atoms, including a range based on any combination of the number of carbon atoms noted herein (for example and without limitation, C₁₋₁₅, C₁₋₁₀, C₁₋₇, C₃₋₁₅, C₃₋₁₀, C₃₋₇, C₅₋₁₅, C₅₋₁₀, C₅₋₇, and the like), which have one or more heteroatoms. In a third embodiment, for example and without limitation, R³ in the compound of Formula I is —CH₃, —C₃H₇, —C₃HD₆, —C₃D₇, —C₅H₉, —C₆H₄Cl, —C₆H₇, —C₆H₁₁, —C₆H₁₂N, —C₆H₁₃, —C₇H₇, —C₁₀H₇ or —C₁₀H₁₁.

The term ‘heteroatoms’ as used herein is not particularly limited and should be known to a person of skill in the art. Heteroatoms include any atom that is not carbon or hydrogen. The term is usually used more specifically to indicate that non-carbon atoms have replaced carbon in the backbone of the molecular structure. In one embodiment, for example and without limitation, the heteroatoms is nitrogen (N), oxygen (O), sulfur (S), phosphorus (P), chlorine (Cl), bromine (Br), iodine (I), or any combination of these.

The term ‘C₁₋₁₅ substituent having one or more heteroatoms’ and like should be clear to a person of skill in the art. In view of the above, the term includes, for example and without limitation, alkyl, alkenyl, alkynyl, cycloalkyl, aryl, heteroaryl, cycloalkyl alkyl, cycloheteroalkyl, alkyl aryl, alkyl heteroaryl and like, that have one or more nitrogen (N), oxygen (O), sulfur (S), phosphorus (P), chlorine (Cl), bromine (Br), iodine (I), or any combination of these.

The term ‘alkyl’ as used herein is not particularly limited and should be known to a person of skill in the art. The term ‘alkyl’ includes an unbranched or branched chain, saturated hydrocarbon residue containing 1 to 15 carbon atoms. The term “C_{1-M} alkyl” refers to an alkyl comprising 1 to M carbon atoms, where M is an integer having the following values: 2, 3, 4, 5, 6, 7, 8, 9, 10, 11, 12, 13, 14, or 15. For example, the term “C₁₋₄ alkyl” refers to an alkyl containing 1 to 4 carbon atoms. Examples of alkyl groups include, but are not limited to, methyl, ethyl, propyl, iso-propyl, butyl, isobutyl, tert-butyl, pentyl, isopentyl, neopentyl, hexyl, heptyl, octyl and like. The term (aryl)alkyl or (heteroaryl)alkyl indicate the alkyl group is optionally substituted by an aryl or a heteroaryl group respectively. Embodiments of an alkyl group having one or more heteroatoms can include, for example and without limitation, —OCH₃, —OCH₂CH₂CH₃, —CH₂CHClCH₃, —CH₂CH₂CH₂NH₂, and the like.

The term ‘alkenyl’ as used herein is not particularly limited and should be known to a person of skill in the art. The term ‘alkenyl’ includes an unbranched or branched chain, hydrocarbon residue containing 1 to 15 carbon atoms having one or more olefinic double bonds. The term “C_{1-N} alkyl” refers to an alkenyl comprising 1 to N carbon atoms, where N is an integer having the following values: 2, 3, 4, 5, 6, 7, 8, 9, 10, 11, 12, 13, 14, or 15. For example, the term “C₂₋₄ alkenyl” refers to an alkenyl moiety containing 2 to 4 carbon atoms. Examples of alkenyl groups include, but are not limited to, vinyl, 1-propenyl, 2-propenyl (allyl) or 2-butenyl (crotyl) and the like. The term (aryl)alkenyl or (heteroaryl)alkenyl indicate the alkenyl group is optionally substituted by an aryl or a heteroaryl group respectively. Embodiments of an alkenyl group having one or more heteroatoms can include, for example and without limitation, —OCH₂CH═CH₂, —CH₂CCl═CH₂, —CH₂CH═CHCH₂NH₂, and the like.

The term ‘alkynyl’ as used herein is not particularly limited and should be known to a person of skill in the art. The term ‘alkynyl’ includes an unbranched or branched chain, hydrocarbon residue containing 1 to 15 carbon atoms having one or more triple bonds. The term “C_{2-N} alkynyl” refers to an alkynyl comprising 2 to N carbon atoms, where N is an integer having the following values: 2, 3, 4, 5, 6, 7, 8, 9, 10, 11, 12, 13, 14, or 15. For example, the term “C₂₋₄ alkynnyl” refers to an alkynyl moiety containing 2 to 4 carbon atoms. Examples of alkynyl groups include, but are not limited to ethynyl, 1-propynyl, 2-propynyl, 1-butynyl, 2-butynyl or 3-butynyl.

The term ‘cycloalkyl’ as used herein is not particularly limited and should be known to a person of skill in the art. The term “cycloalkyl” refers to a saturated carbocyclic ring having 3 to 8 carbon atoms. Non-limiting examples include cyclopropyl, cyclobutyl, cyclopentyl, cyclohexyl, cycloheptyl or cyclooctyl. The term “C₃₋₇ cycloalkyl” as used herein refers to a cycloalkyl having 3 to 7 carbons in the carbocyclic ring. In addition, terms, such as ‘cycloalkyl alkyl’, ‘cycloalkyl alkenyl’ or ‘cycloalkyl alkynyl’ refer to a cycloalkyl having a alkyl, alkenyl or alkynyl substituent.

The term ‘aryl’ as used herein is not particularly limited and should be known to a person of skill in the art. The term “aryl,” as used herein, and unless otherwise specified, refers to substituted or unsubstituted phenyl (Ph), biphenyl, or naphthyl. The aryl group can be substituted with one or more moieties, and include, for example and without limitation, hydroxyl, F, Cl, Br, I, amino, alkylamino, arylamino, alkoxy, aryloxy, nitro, cyano, sulfonic acid, sulfate, phosphonic acid, phosphate, and phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999 (incorporated herein by reference).

The terms “alkaryl” or “alkylaryl” as used herein are not particularly limited and should be known to a person of skill in the art. The terms “alkaryl” or “alkylaryl” refer to an alkyl group with an aryl substituent, such as benzyl. The terms “aralkyl” or “arylalkyl” refer to an aryl group with an alkyl substituent.

The term ‘heterocycle’ as used herein is not particularly limited and should be known to a person of skill in the art. The term “heterocycle” refers to an unsubstituted or substituted heterocycle containing carbon, hydrogen, and at least one of N, O, and S, where the C and N can be trivalent or tetravalent, i.e., sp² or sp³-hybridized. Examples of heterocycles include, for example and without limitation, aziridine, azetidine, pyrrolidine, piperidine, imidazole, oxazole, piperazine, and the like.

The term ‘heteroaryl’ as used herein is not particularly limited and should be known to a person of skill in the art. The ‘heteroaryl’ relates to an aryl group having at least one of N, O, and S. Examples of heteroaryls include, for example and without limitation, furan, oxazole, thiophene, 1,2,3-triazole, 1,2,4-triazine, 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, and the like.

The term ‘PG’ stands for protecting group. Protecting groups used with the compounds disclosed herein are not particularly limited, and should be known to a person of skill in the art, or can be determined. A protecting group is introduced into a molecule by chemical modification of a functional group to obtain chemoselectivity in a subsequent chemical reaction.

The term ‘PG_{N}’ as used herein relates to a protecting group bonded to a nitrogen, for example, an amine protecting group. Amine protecting groups as disclosed herein are not particularly limited and should be known to a person of skill in the art. Non-limiting examples of an amine protecting group include fluorenylmethoxycarbonyl protecting group (Fmoc), tert-butoxycarbonyl (BOC), carbobenzyloxy (Cbz), trifluoroacetamide, phthalimide, trityl (Tr), monomethoxytrityl (MMT), dimethoxytrityl (DMT), or benzylideneamine.

The term ‘PG_{O}’ as used herein relates to a protecting group bonded to an oxygen, for example, an alcohol protecting group, while ‘PG_{O1}’ relates to a first protecting group boned to oxygen and ‘PG_{O2}’ relates to a second protecting group bonded to oxygen. The selection of ‘PG_{O1}’ and ‘PG_{O2}’ can vary depending upon design and synthetic requirements. Further ‘PG_{O1}’ and ‘PG_{O2}’ can be the same or different. Alcohol protecting groups as disclosed herein are not particularly limited and should be known to a person of skill in the art. Non-limiting examples of an alcohol protecting group include benzyl (Bn), t-butyldimethylsilyl (TBDMS), t-butyldiphenylsilyl (TBDPS), acetyl (Ac), pivaloyl (Piv), or Benzyl (Bz), trityl (Tr), monomethoxytrityl (MMT), dimethoxytrityl (DMT). In addition, ‘PG_{O1}’ and ‘PG_{O2}’ can together form, for example and without limitation,

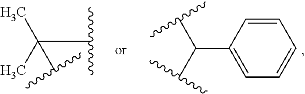

which can be used to protect a diol.

The term ‘prodrug’ as used herein is not particularly limited and should be known to a person of skill in the art. A prodrug is a compound that undergoes biotransformation before exhibiting pharmacological effects. A prodrug is a compound that, after intake, is metabolized (i.e., converted within the body) into a pharmacologically active drug. Instead of administering a drug directly, a corresponding prodrug can be used to improve how the drug is absorbed, distributed, metabolized, and excreted (ADME). Prodrugs are often designed to improve bioavailability when a drug itself is poorly absorbed from the gastrointestinal tract. A prodrug may be used to improve how selectively the drug interacts with cells or processes that are not its intended target. This reduces adverse or unintended effects of a drug. The compounds of Formula I disclosed herein are analogs of P5P that can be prodrugs for treatment or prevention of diseases associated with P5P. Studies, as described herein, show improved bioavailability of P5P using compounds of Formula I, and as such can function as potential prodrugs.

Using the method disclosed herein, a number of compounds encompassed by the compound of Formula I have been prepared. Embodiments of pyridoxamine prodrugs encompassed by the compound of Formula I are shown in Table 1.

TABLE 1
Embodiments of pyridoxamine prodrugs
14

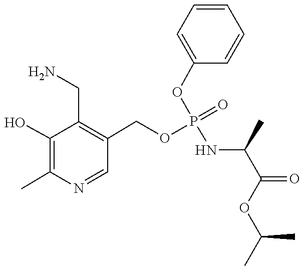

Pyridoxamine prodrug 14

| | |
|---|---|
| 14i | 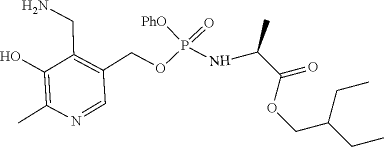 [SMILES: CCC(CC)COC(=O)[C@H](C)NP(=O)(OCc1cnc(C)c(O)c1CN)Oc1ccccc1] |
| 14ii | 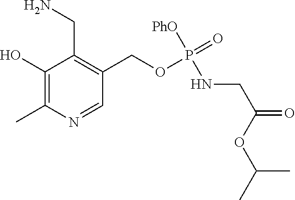 [SMILES: Cc1ncc(COP(=O)(NCC(=O)OC(C)C)Oc2ccccc2)c(CN)c1O] |
| 14iii | 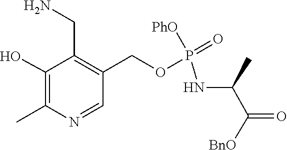 [SMILES: Cc1ncc(COP(=O)(N[C@@H](C)C(=O)OCc2ccccc2)Oc2ccccc2)c(CN)c1O] |
| 14iv | 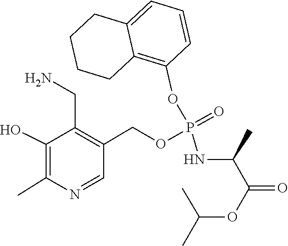 [SMILES: Cc1ncc(COP(=O)(N[C@@H](C)C(=O)OC(C)C)Oc2cccc3c2CCCC3)c(CN)c1O] |
| 14v | 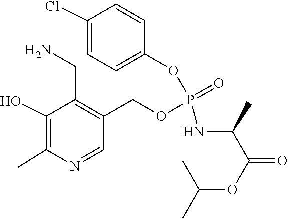 [SMILES: Cc1ncc(COP(=O)(N[C@@H](C)C(=O)OC(C)C)Oc2ccc(Cl)cc2)c(CN)c1O] |
| 14vi | 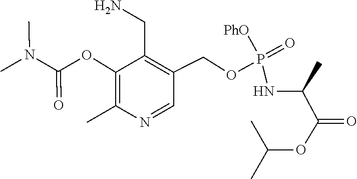 [SMILES: Cc1ncc(COP(=O)(N[C@@H](C)C(=O)OC(C)C)Oc2ccccc2)c(CN)c1OC(=O)N(C)C] |
| 14vii | 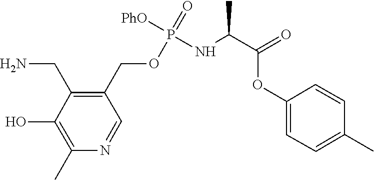 [SMILES: Cc1ccc(OC(=O)[C@H](C)NP(=O)(OCc2cnc(C)c(O)c2CN)Oc2ccccc2)cc1] |
| 14viii | 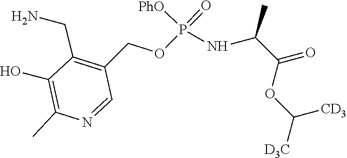 [SMILES: [2H]C([2H])([2H])C(OC(=O)[C@H](C)NP(=O)(OCc1cnc(C)c(O)c1CN)Oc1ccccc1)C([2H])([2H])[2H]] |
| 14ix | 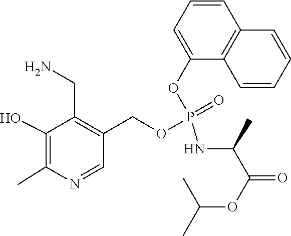 [SMILES: Cc1ncc(COP(=O)(N[C@@H](C)C(=O)OC(C)C)Oc2cccc3ccccc23)c(CN)c1O] |
| 14x | 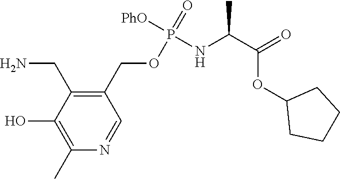 [SMILES: Cc1ncc(COP(=O)(N[C@@H](C)C(=O)OC2CCCC2)Oc2ccccc2)c(CN)c1O] |
| 14xi | 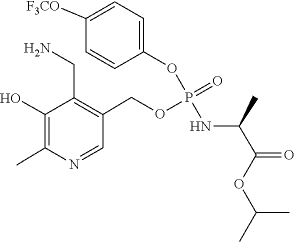 [SMILES: Cc1ncc(COP(=O)(N[C@@H](C)C(=O)OC(C)C)Oc2ccc(OC(F)(F)F)cc2)c(CN)c1O] |
| 14xii | 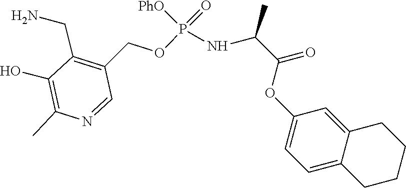 [SMILES: Cc1ncc(COP(=O)(N[C@@H](C)C(=O)Oc2ccc3c(c2)CCCC3)Oc2ccccc2)c(CN)c1O] |
| 14xiii | 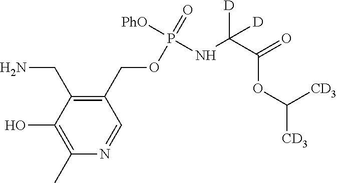 [SMILES: [2H]C([2H])(NP(=O)(OCc1cnc(C)c(O)c1CN)Oc1ccccc1)C(=O)OC(C([2H])([2H])[2H])C([2H])([2H])[2H]] |
| 14xiv | 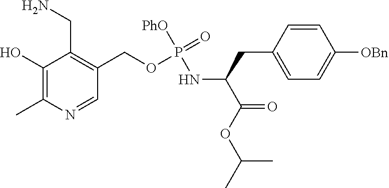 [SMILES: Cc1ncc(COP(=O)(N[C@@H](Cc2ccc(OCc3ccccc3)cc2)C(=O)OC(C)C)Oc2ccccc2)c(CN)c1O] |
| 14v | 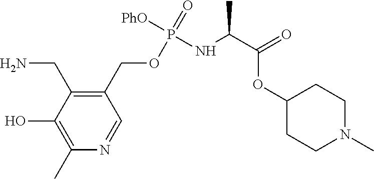 [SMILES: Cc1ncc(COP(=O)(N[C@@H](C)C(=O)OC2CCN(C)CC2)Oc2ccccc2)c(CN)c1O] |
| 14vi | 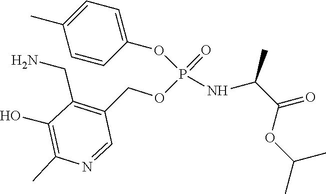 [SMILES: Cc1ccc(OP(=O)(N[C@@H](C)C(=O)OC(C)C)OCc2cnc(C)c(O)c2CN)cc1] |
| 14vii | 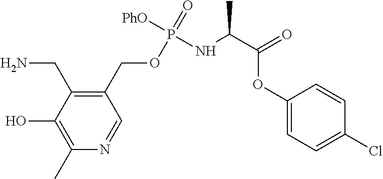 [SMILES: Cc1ncc(COP(=O)(N[C@@H](C)C(=O)Oc2ccc(Cl)cc2)Oc2ccccc2)c(CN)c1O] |
| 14viii | 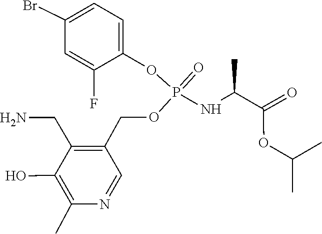 [SMILES: Cc1ncc(COP(=O)(N[C@@H](C)C(=O)OC(C)C)Oc2ccc(Br)cc2F)c(CN)c1O] |

Embodiments of pyridoxine prodrugs encompassed by the compound of Formula I are shown in Table 2.

TABLE 2
Embodiments of pyridoxine prodrugs

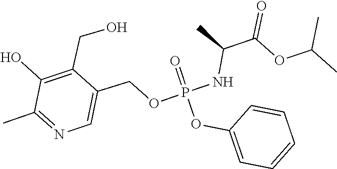 [SMILES: Cc1ncc(COP(=O)(N[C@@H](C)C(=O)OC(C)C)Oc2ccccc2)c(CO)c1O]

Pyridoxine prodrug 15

| | |
|---|---|
| 15i | 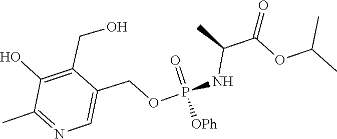 [SMILES: Cc1ncc(CO[P@@](=O)(N[C@@H](C)C(=O)OC(C)C)Oc2ccccc2)c(CO)c1O] |
| 15ii | 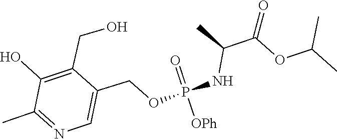 |
| 15iii | 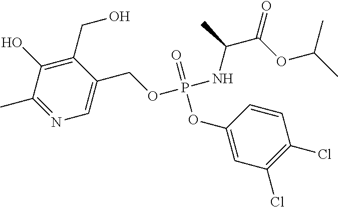 [SMILES: Cc1ncc(COP(=O)(N[C@@H](C)C(=O)OC(C)C)Oc2ccc(Cl)c(Cl)c2)c(CO)c1O] |
| 15iv | 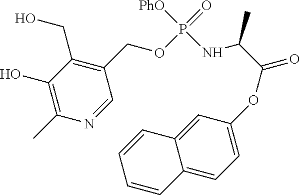 [SMILES: Cc1ncc(COP(=O)(N[C@@H](C)C(=O)Oc2ccc3ccccc3c2)Oc2ccccc2)c(CO)c1O] |
| 15v | 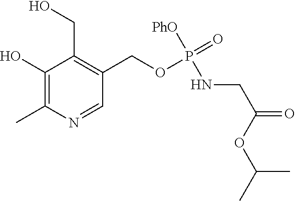 [SMILES: Cc1ncc(COP(=O)(NCC(=O)OC(C)C)Oc2ccccc2)c(CO)c1O] |
| 15vi | 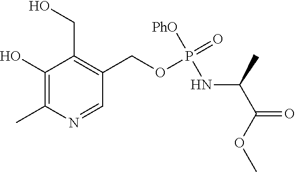 [SMILES: COC(=O)[C@H](C)NP(=O)(OCc1cnc(C)c(O)c1CO)Oc1ccccc1] |
| 15vii | 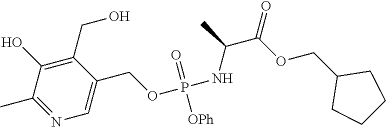 [SMILES: Cc1ncc(COP(=O)(N[C@@H](C)C(=O)OCC2CCCC2)Oc2ccccc2)c(CO)c1O] |
| 15viii | 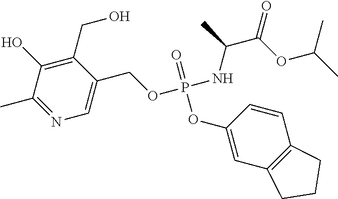 [SMILES: Cc1ncc(COP(=O)(N[C@@H](C)C(=O)OC(C)C)Oc2ccc3c(c2)CCC3)c(CO)c1O] |
| 15ix | 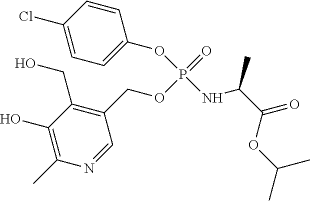 [SMILES: Cc1ncc(COP(=O)(N[C@@H](C)C(=O)OC(C)C)Oc2ccc(Cl)cc2)c(CO)c1O] |
| 15x | 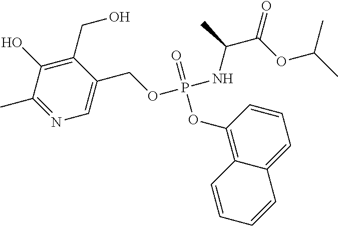 [SMILES: Cc1ncc(COP(=O)(N[C@@H](C)C(=O)OC(C)C)Oc2cccc3ccccc23)c(CO)c1O] |
| 15xi | 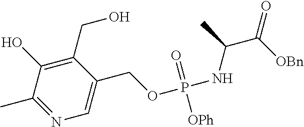 [SMILES: Cc1ncc(COP(=O)(N[C@@H](C)C(=O)OCc2ccccc2)Oc2ccccc2)c(CO)c1O] |
| 15xii | 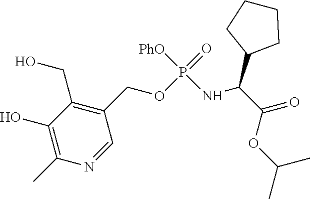 [SMILES: Cc1ncc(COP(=O)(N[C@H](C(=O)OC(C)C)C2CCCC2)Oc2ccccc2)c(CO)c1O] |
| 15xiii | 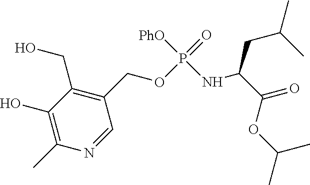 [SMILES: Cc1ncc(COP(=O)(N[C@@H](CC(C)C)C(=O)OC(C)C)Oc2ccccc2)c(CO)c1O] |
| 15xiv | 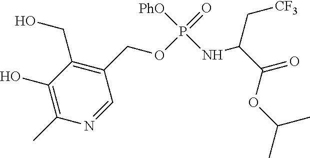 [SMILES: Cc1ncc(COP(=O)(NC(CC(F)(F)F)C(=O)OC(C)C)Oc2ccccc2)c(CO)c1O] |
| 15xv | 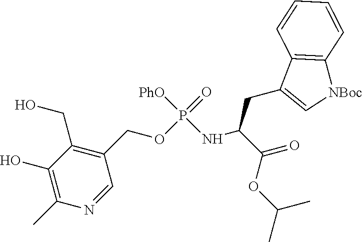 [SMILES: Cc1ncc(COP(=O)(N[C@@H](Cc2cn(C(=O)OC(C)(C)C)c3ccccc23)C(=O)OC(C)C)Oc2ccccc2)c(CO)c1O] |
| 15xvi | 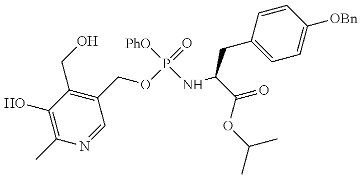 [SMILES: Cc1ncc(COP(=O)(N[C@@H](Cc2ccc(OCc3ccccc3)cc2)C(=O)OC(C)C)Oc2ccccc2)c(CO)c1O] |
| 15xvii | 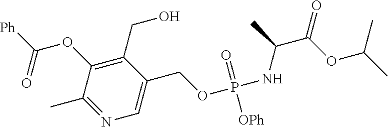 [SMILES: Cc1ncc(COP(=O)(N[C@@H](C)C(=O)OC(C)C)Oc2ccccc2)c(CO)c1OC(=O)c1ccccc1] |
| 15xviii | 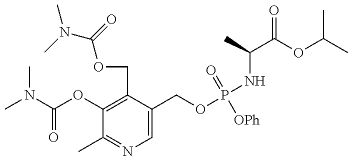 [SMILES: Cc1ncc(COP(=O)(N[C@@H](C)C(=O)OC(C)C)Oc2ccccc2)c(COC(=O)N(C)C)c1OC(=O)N(C)C] |
| 15xix | 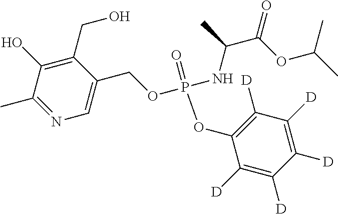 [SMILES: [2H]c1c([2H])c([2H])c(OP(=O)(N[C@@H](C)C(=O)OC(C)C)OCc2cnc(C)c(O)c2CO)c([2H])c1[2H]] |
| 15xx | 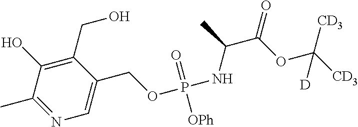 [SMILES: [2H]C([2H])([2H])C([2H])(OC(=O)[C@H](C)NP(=O)(OCc1cnc(C)c(O)c1CO)Oc1ccccc1)C([2H])([2H])[2H]] |
| 15xxi | 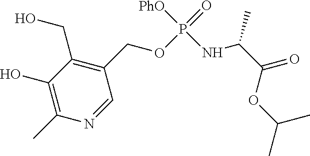 [SMILES: Cc1ncc(COP(=O)(N[C@H](C)C(=O)OC(C)C)Oc2ccccc2)c(CO)c1O] |
| 15xxii | 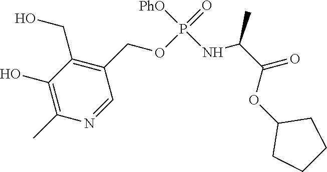 [SMILES: Cc1ncc(COP(=O)(N[C@@H](C)C(=O)OC2CCCC2)Oc2ccccc2)c(CO)c1O] |
| 15xxiii | 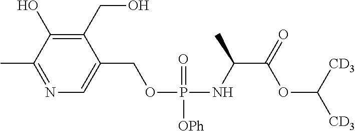 [SMILES: [2H]C([2H])([2H])C(OC(=O)[C@H](C)NP(=O)(OCc1cnc(C)c(O)c1CO)Oc1ccccc1)C([2H])([2H])[2H]] |
| 15xxiv | 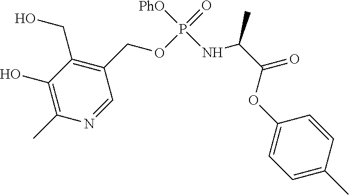 [SMILES: Cc1ccc(OC(=O)[C@H](C)NP(=O)(OCc2cnc(C)c(O)c2CO)Oc2ccccc2)cc1] |
| 15xxv | 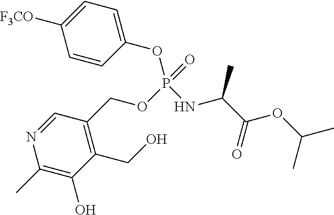 [SMILES: Cc1ncc(COP(=O)(N[C@@H](C)C(=O)OC(C)C)Oc2ccc(OC(F)(F)F)cc2)c(CO)c1O] |
| 15xxvi | 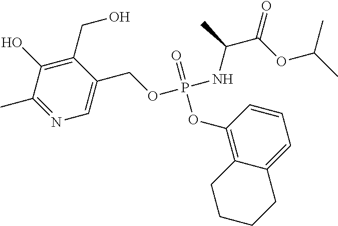 [SMILES: Cc1ncc(COP(=O)(N[C@@H](C)C(=O)OC(C)C)Oc2cccc3c2CCCC3)c(CO)c1O] |
| 15xxvii | 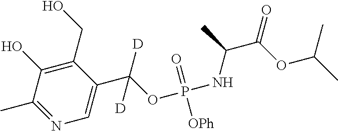 [SMILES: [2H]C([2H])(OP(=O)(N[C@@H](C)C(=O)OC(C)C)Oc1ccccc1)c1cnc(C)c(O)c1CO] |
| 15xxviii | 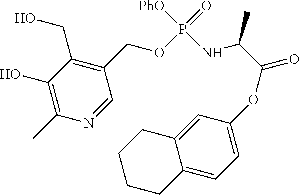 [SMILES: Cc1ncc(COP(=O)(N[C@@H](C)C(=O)Oc2ccc3c(c2)CCCC3)Oc2ccccc2)c(CO)c1O] |
| 15xxix | 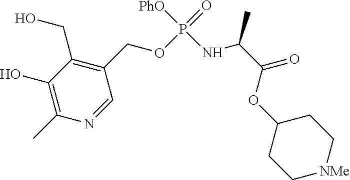 [SMILES: Cc1ncc(COP(=O)(N[C@@H](C)C(=O)OC2CCN(C)CC2)Oc2ccccc2)c(CO)c1O] |
| 15xxx | 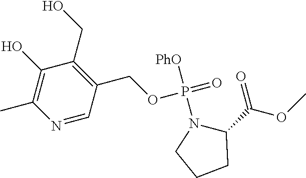 |
| 15xxxi | 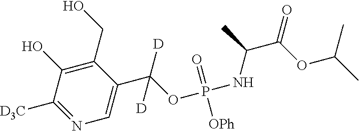 [SMILES: [2H]C([2H])([2H])c1ncc(C([2H])([2H])OP(=O)(N[C@@H](C)C(=O)OC(C)C)Oc2ccccc2)c(CO)c1O] |
| 15xxxii | 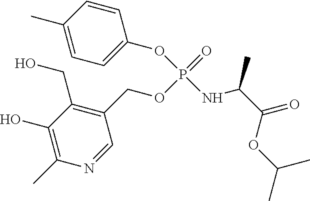 [SMILES: Cc1ccc(OP(=O)(N[C@@H](C)C(=O)OC(C)C)OCc2cnc(C)c(O)c2CO)cc1] |
| 15xxxiii | 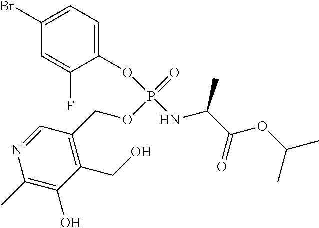 [SMILES: Cc1ncc(COP(=O)(N[C@@H](C)C(=O)OC(C)C)Oc2ccc(Br)cc2F)c(CO)c1O] |
| 15xxxiv | 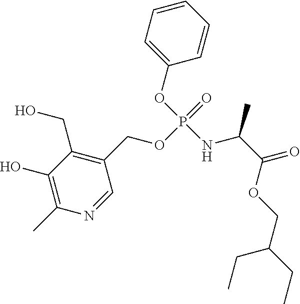 [SMILES: CCC(CC)COC(=O)[C@H](C)NP(=O)(OCc1cnc(C)c(O)c1CO)Oc1ccccc1] |

Embodiments of pyridoxal prodrugs encompassed by the compound of Formula I are shown in Table 3.

TABLE 3
Embodiments of pyridoxal prodrugs

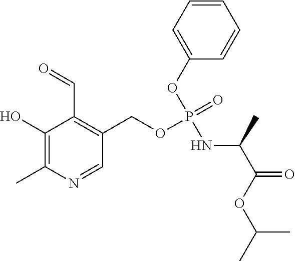 [SMILES: Cc1ncc(COP(=O)(N[C@@H](C)C(=O)OC(C)C)Oc2ccccc2)c(C=O)c1O]

Pyridoxal prodrug 16

| | |
|---|---|
| 16i | 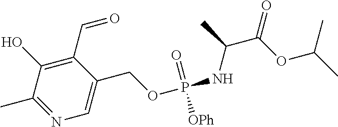 [SMILES: Cc1ncc(CO[P@@](=O)(N[C@@H](C)C(=O)OC(C)C)Oc2ccccc2)c(C=O)c1O] |
| 16ii | 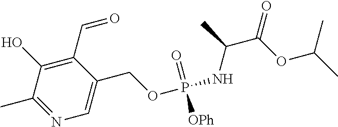 [SMILES: Cc1ncc(CO[P@](=O)(N[C@@H](C)C(=O)OC(C)C)Oc2ccccc2)c(C=O)c1O] |
| 16iii | 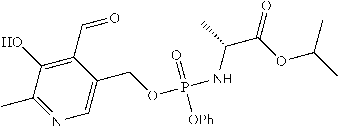 [SMILES: Cc1ncc(COP(=O)(N[C@H](C)C(=O)OC(C)C)Oc2ccccc2)c(C=O)c1O] |
| 16iv | 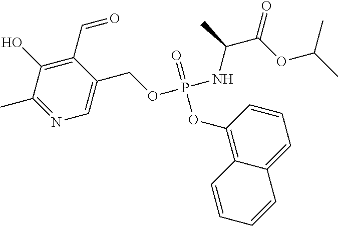 [SMILES: Cc1ncc(COP(=O)(N[C@@H](C)C(=O)OC(C)C)Oc2cccc3ccccc23)c(C=O)c1O] |
| 16v | 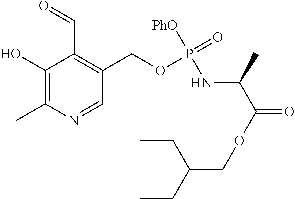 [SMILES: CCC(CC)COC(=O)[C@H](C)NP(=O)(OCc1cnc(C)c(O)c1C=O)Oc1ccccc1] |
| 16vi | 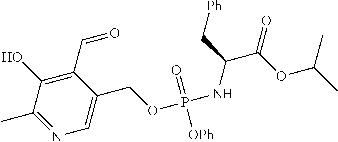 [SMILES: Cc1ncc(COP(=O)(N[C@@H](Cc2ccccc2)C(=O)OC(C)C)Oc2ccccc2)c(C=O)c1O] |
| 16vii | 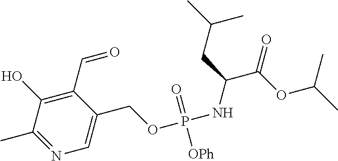 [SMILES: Cc1ncc(COP(=O)(N[C@@H](CC(C)C)C(=O)OC(C)C)Oc2ccccc2)c(C=O)c1O] |
| 16viii | 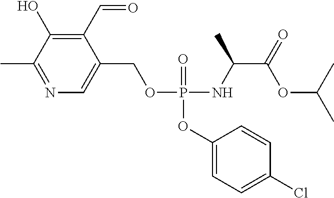 [SMILES: Cc1ncc(COP(=O)(N[C@@H](C)C(=O)OC(C)C)Oc2ccc(Cl)cc2)c(C=O)c1O] |
| 16ix | 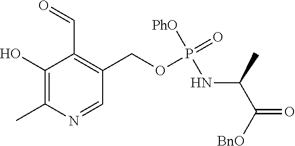 [SMILES: Cc1ncc(COP(=O)(N[C@@H](C)C(=O)OCc2ccccc2)Oc2ccccc2)c(C=O)c1O] |
| 16x | 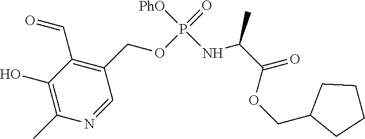 [SMILES: Cc1ncc(COP(=O)(N[C@@H](C)C(=O)OCC2CCCC2)Oc2ccccc2)c(C=O)c1O] |
| 16xi | 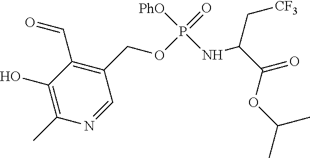 [SMILES: Cc1ncc(COP(=O)(NC(CC(F)(F)F)C(=O)OC(C)C)Oc2ccccc2)c(C=O)c1O] |
| 16xii | 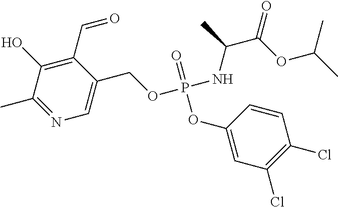 [SMILES: Cc1ncc(COP(=O)(N[C@@H](C)C(=O)OC(C)C)Oc2ccc(Cl)c(Cl)c2)c(C=O)c1O] |
| 16xiii | 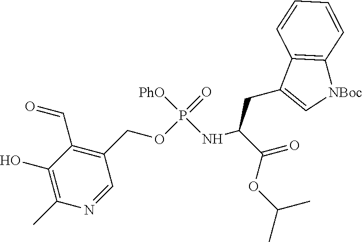 [SMILES: Cc1ncc(COP(=O)(N[C@@H](Cc2cn(C(=O)OC(C)(C)C)c3ccccc23)C(=O)OC(C)C)Oc2ccccc2)c(C=O)c1O] |
| 16xiv | 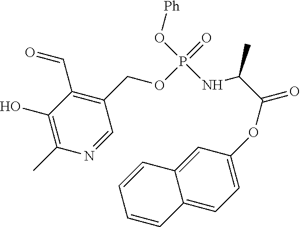 [SMILES: Cc1ncc(COP(=O)(N[C@@H](C)C(=O)Oc2ccc3ccccc3c2)Oc2ccccc2)c(C=O)c1O] |
| 16xv | 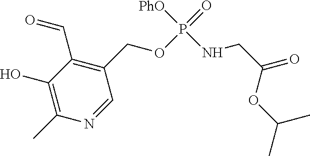 [SMILES: Cc1ncc(COP(=O)(NCC(=O)OC(C)C)Oc2ccccc2)c(C=O)c1O] |
| 16xvi | 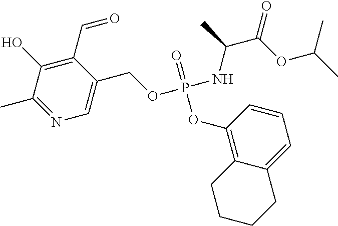 [SMILES: Cc1ncc(COP(=O)(N[C@@H](C)C(=O)OC(C)C)Oc2cccc3c2CCCC3)c(C=O)c1O] |
| 16xvii | 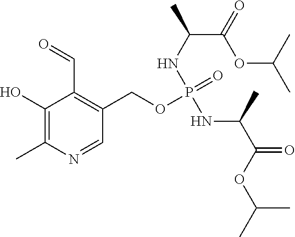 [SMILES: Cc1ncc(COP(=O)(N[C@@H](C)C(=O)OC(C)C)N[C@@H](C)C(=O)OC(C)C)c(C=O)c1O] |
| 16xviii | 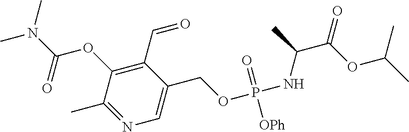 [SMILES: Cc1ncc(COP(=O)(N[C@@H](C)C(=O)OC(C)C)Oc2ccccc2)c(C=O)c1OC(=O)N(C)C] |
| 16xix | 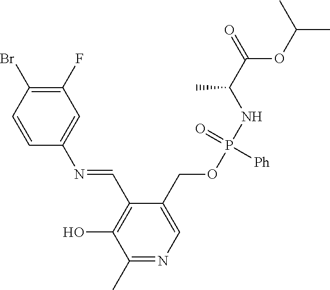 [SMILES: Cc1ncc(COP(=O)(N[C@H](C)C(=O)OC(C)C)c2ccccc2)c(/C=N/c2ccc(Br)c(F)c2)c1O] |
| 16xx | 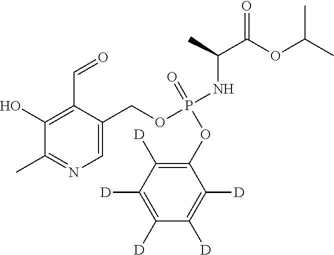 [SMILES: [2H]c1c([2H])c([2H])c(OP(=O)(N[C@@H](C)C(=O)OC(C)C)OCc2cnc(C)c(O)c2C=O)c([2H])c1[2H]] |
| 16xxi | 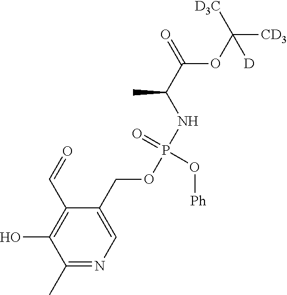 [SMILES: [2H]C([2H])([2H])C([2H])(OC(=O)[C@H](C)NP(=O)(OCc1cnc(C)c(O)c1C=O)Oc1ccccc1)C([2H])([2H])[2H]] |
| 16xxii | 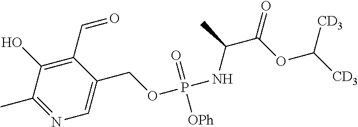 [SMILES: [2H]C([2H])([2H])C(OC(=O)[C@H](C)NP(=O)(OCc1cnc(C)c(O)c1C=O)Oc1ccccc1)C([2H])([2H])[2H]] |
| 16xxiii | 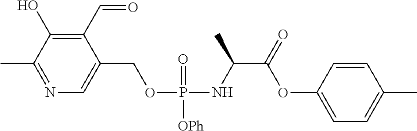 |
| 16xxiv | 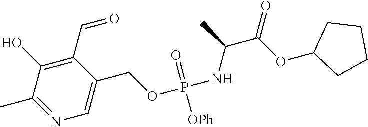 [SMILES: Cc1ncc(COP(=O)(N[C@@H](C)C(=O)OC2CCCC2)Oc2ccccc2)c(C=O)c1O] |
| 16xxv | 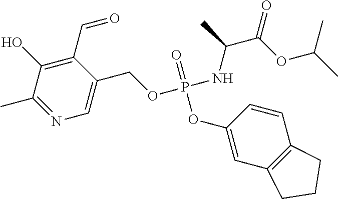 [SMILES: Cc1ncc(COP(=O)(N[C@@H](C)C(=O)OC(C)C)Oc2ccc3c(c2)CCC3)c(C=O)c1O] |
| 16xxvi | 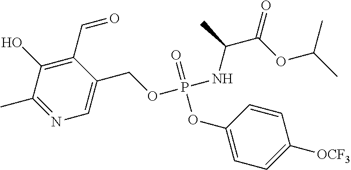 [SMILES: Cc1ncc(COP(=O)(N[C@@H](C)C(=O)OC(C)C)Oc2ccc(OC(F)(F)F)cc2)c(C=O)c1O] |
| 16xxvii | 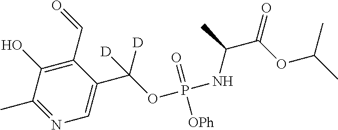 [SMILES: [2H]C([2H])(OP(=O)(N[C@@H](C)C(=O)OC(C)C)Oc1ccccc1)c1cnc(C)c(O)c1C=O] |
| 16xxviii | 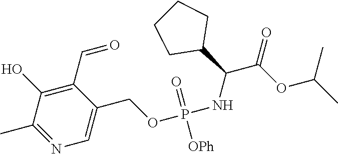 [SMILES: Cc1ncc(COP(=O)(N[C@H](C(=O)OC(C)C)C2CCCC2)Oc2ccccc2)c(C=O)c1O] |
| 16xxix | 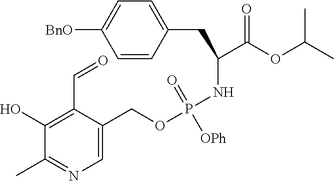 [SMILES: Cc1ncc(COP(=O)(N[C@@H](Cc2ccc(OCc3ccccc3)cc2)C(=O)OC(C)C)Oc2ccccc2)c(C=O)c1O] |
| 16xxx | 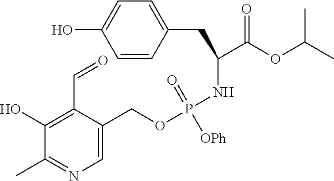 [SMILES: Cc1ncc(COP(=O)(N[C@@H](Cc2ccc(O)cc2)C(=O)OC(C)C)Oc2ccccc2)c(C=O)c1O] |
| 16xxxxi | 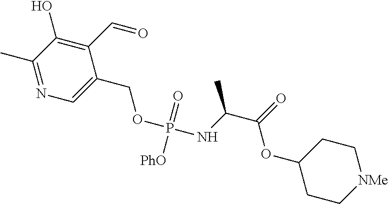 [SMILES: Cc1ncc(COP(=O)(N[C@@H](C)C(=O)OC2CCN(C)CC2)Oc2ccccc2)c(C=O)c1O] |
| 16xxxii | 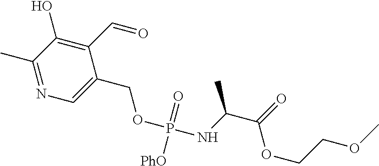 |
| 16xxxiii | 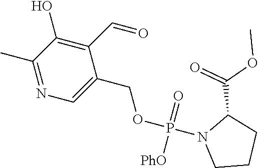 [SMILES: COC(=O)[C@@H]1CCCN1P(=O)(OCc1cnc(C)c(O)c1C=O)Oc1ccccc1] |
| 16xxxiv | 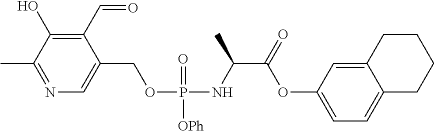 [SMILES: Cc1ncc(COP(=O)(N[C@@H](C)C(=O)Oc2ccc3c(c2)CCCC3)Oc2ccccc2)c(C=O)c1O] |
| 16xxxv | 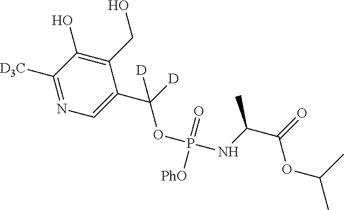 [SMILES: [2H]C([2H])([2H])c1ncc(C([2H])([2H])OP(=O)(N[C@@H](C)C(=O)OC(C)C)Oc2ccccc2)c(CO)c1O] |
| 16xxxvi | 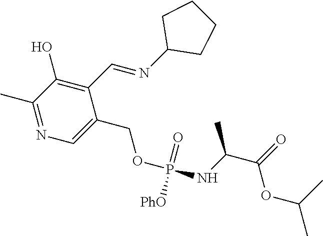 [SMILES: Cc1ncc(CO[P@@](=O)(N[C@@H](C)C(=O)OC(C)C)Oc2ccccc2)c(/C=N/C2CCCC2)c1O] |
| 16xxxvii | 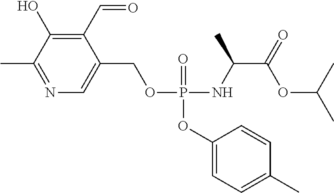 [SMILES: Cc1ccc(OP(=O)(N[C@@H](C)C(=O)OC(C)C)OCc2cnc(C)c(O)c2C=O)cc1] |
| 16xxxviii | 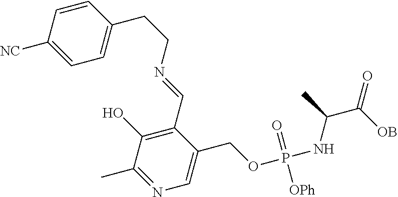 |
| 16xxxix | 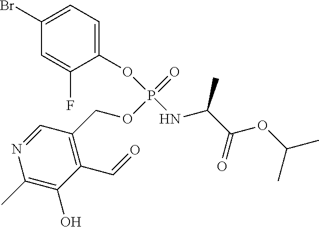 [SMILES: Cc1ncc(COP(=O)(N[C@@H](C)C(=O)OC(C)C)Oc2ccc(Br)cc2F)c(C=O)c1O] |
| 16xl | 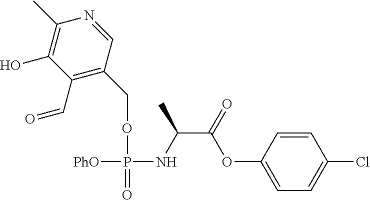 [SMILES: Cc1ncc(COP(=O)(N[C@@H](C)C(=O)Oc2ccc(Cl)cc2)Oc2ccccc2)c(C=O)c1O] |

### Process for Preparation

In an aspect, the specification relates to a process for preparation of a compound, its stereoisomer, salt, hydrate, solvate, isotope or crystalline form thereof, of Formula I:

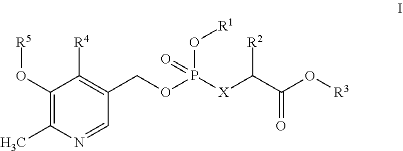

the process containing the step of reacting the compound of Formula II with a compound of Formula III, in the presence of a base, to form the compound of Formula I

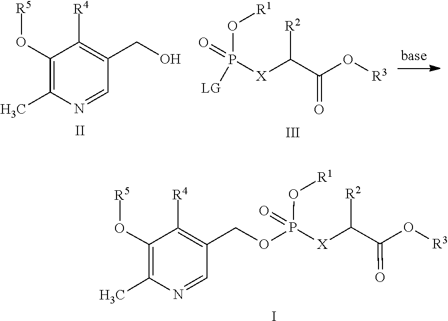

wherein X, R¹, R², R³, R⁴ and R⁵ are as described herein.

The base used for carrying out the reaction is not particularly limited, and should be known to a person of skill in the art, or can be determined. In one embodiment, for example and without limitation, the base is an organic base. In another embodiment, for example and without limitation, the base is diisopropylamine (DIPEA), pyridine, dimethylamine, imidazole, benzimidazole, and the like.

The reaction can be carried out in the presence of a solvent. The solvent used is not particularly limited, and should be known to a person of skill in the art, or can be determined. The solvent selected should avoid reacting with the compounds of Formula II and III. In one embodiment, for example and without limitation, the solvent is an organic solvent. In another embodiment, for example and without limitation, the solvent can be a polar or non-polar solvent. In a further embodiment, for example and without limitation, the solvent can be an aprotic solvent. In a still further embodiment, for example and without limitation, the solvent is dichloromethane, acetone, acetonitrile, dimethylformamide, ethyl acetate, dimethyl sulfoxide, pyridine or tetrahydrofuran (THF).

Based on the disclosure herein, compounds of the present application can be synthesized in a variety of ways using commercially available starting materials, compounds known in the literature, or from readily prepared intermediates. By employing standard synthetic methods and procedures, those skilled in the art of synthesis can successfully prepare the compounds in the present application. Standard synthetic methods and procedures for the preparation of organic molecules and functional group transformations and manipulations can be obtained from relevant literature or from standard textbooks in the field. There exist numerous examples of text books on the subject with Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & 15 Sons: New York, 1999, being a good example (all incorporated herein by reference).

Suitable synthetic routes are depicted in General Schemes 1 and 2 to illustrate the general procedures for the preparation of compounds of the present application. These schemes generally provide the desired final compound but it may be desirable in certain examples to further convert the compound to a pharmaceutically acceptable salt, ester, carbonate, carbamate, or imine. The present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley30 Interscience, 1994) (incorporated herein by reference).

Description of General Scheme 1: The dichlorophosphate species 1 was reacted with the salt (HCl or TFA) of an amine 2 in the presence of a base to form species 3 which is not isolated. To the reaction mixture, 4-nitrophenol and additional base were added. The mixture is then stirred to form compound 5. After the purification of compound 5, it is reacted with compound 6 in the presence of a base to give compound 7. The de-protection of purified compound 7 gives compound 8 which is then oxidized to give compound 9.

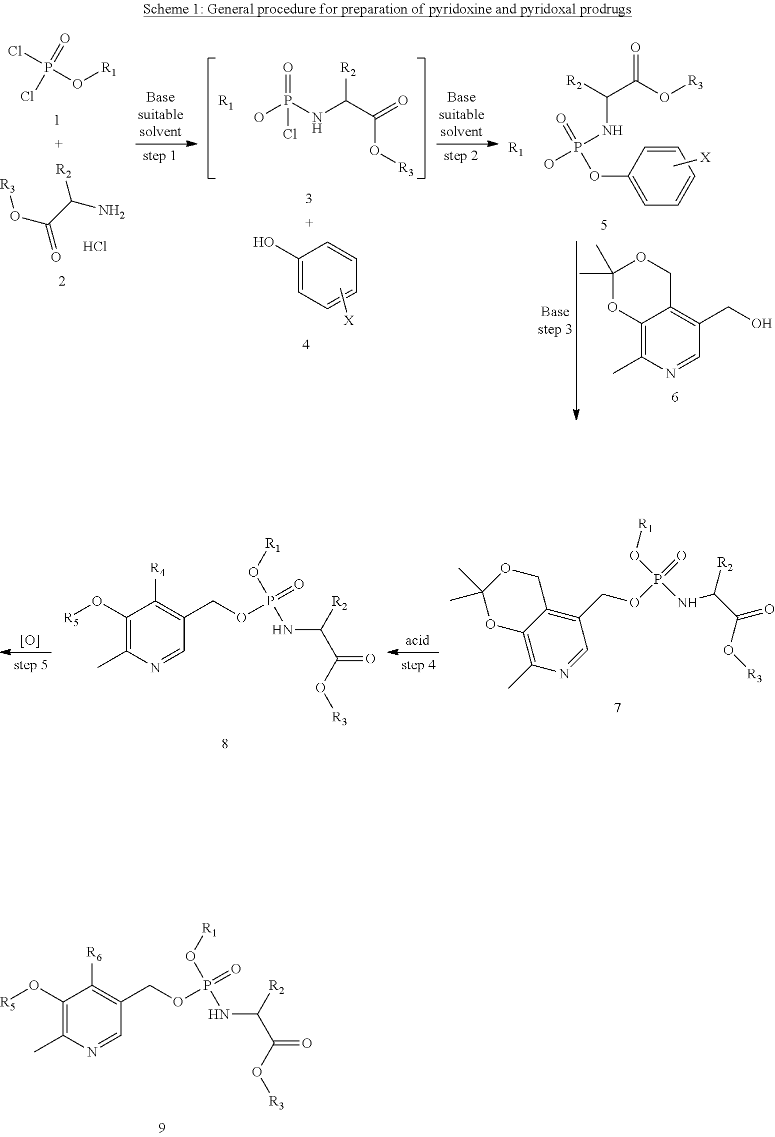

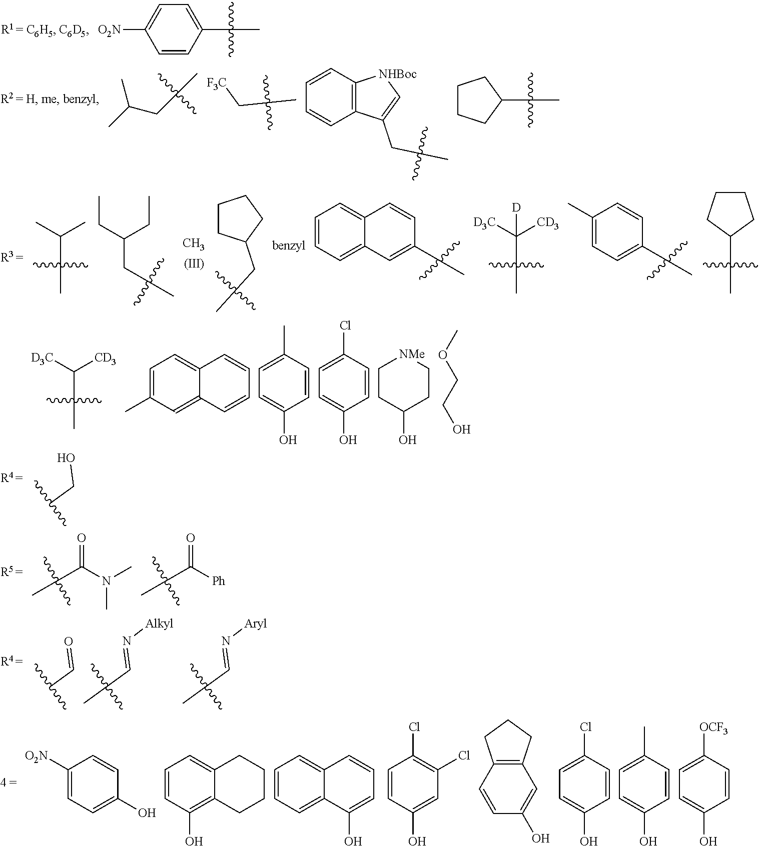

Compounds of formula 7, 8 and 9 shown above in Scheme 1 represent embodiments of the compound of structural formula I, as described herein. Compound of formula 6 shown above in Scheme 1 represents an embodiment of the compound of structural formula II, as described herein. Compounds of formula 3 and 5 shown above in Scheme 1 represent embodiments of the compound of structural formula III or V (as described herein), depending upon the substituent present. Compound of formula 1 shown above in Scheme 1 represents an embodiment of the compound of structural formula VI, as described herein. Compound of formula 2 shown above in Scheme 1 represents an embodiment of the compound of structural formula VII, as described herein. Compound of formula 4 shown above in Scheme 1 represents an embodiment of the compound of structural formula IV, as described herein. In addition, disclosed in Scheme 1 are exemplary non-limiting embodiments of the compound of structural formula IV. Also shown in Scheme 1 are exemplary non-limiting embodiments of substituents R¹, R², R³, R⁴, R⁵ and R⁶.

Description of General Scheme 2: The protected alcohol 11 was reacted with species 5 in the presence of a base and MgCl₂ to give compound 12. The removal of protective groups then furnishes 13. Using these general schemes 1 and 2, some preferred embodiments of the present application relating to the compounds having one of the structures below or being one of the compounds below were prepared.

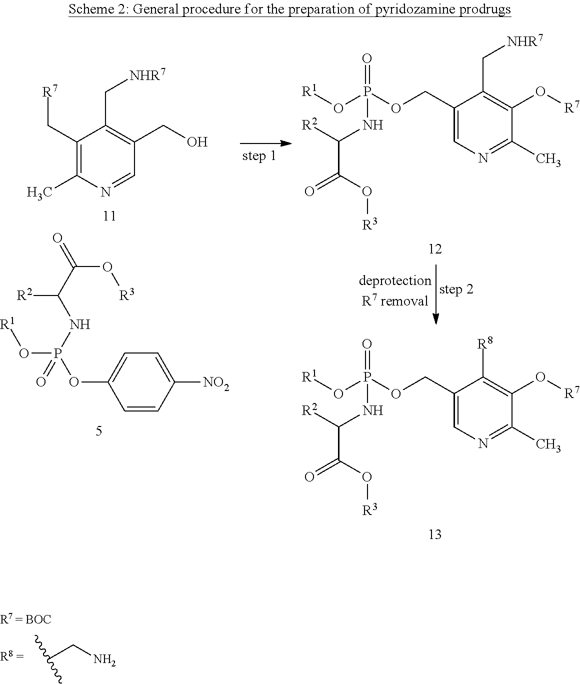

Compound of formula 11 shown above in Scheme 2 represents an embodiment of the compound of structural formula II, as described herein. Compound of formula 5 shown above in Scheme 2 represents an embodiment of the compound of structural formula III or V, as described herein. Compounds of formula 12 and 13 shown above in Scheme 2 represent embodiments of the compound of structural formula I, as described herein.

Based on the disclosure herein, and the general process described in Schemes 1 and 2, a number of compounds were prepared. The compounds of Formula I prepared and tested were of 95% purity or better by HPLC analysis.

### Dosage, Administration and Use

In a third aspect, the specification relates to a composition comprising:
a carrier, diluent or excipient, and
a compound, its stereoisomer, salt, hydrate, solvate, isotope or crystalline form thereof, represented by formula I:

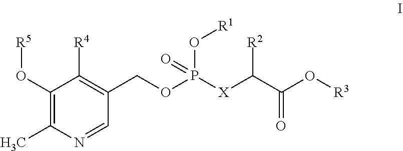

wherein X, R¹, R², R³, R⁴ and R⁵ are as described herein.

The route of administration of the composition (or pharmaceutical composition) is not particularly limited, and should be known to a person of skill in the art, or can be determined. In one embodiment, for example and without limitation, the composition can be formulated for enteral route or parenteral route of administration.

In one embodiment, for example and without limitation, the compounds disclosed herein may be formulated in a wide variety of oral administration dosage forms and carriers. Oral administration can be in the form of tablets, coated tablets, hard and soft gelatin capsules, solutions, emulsions, syrups, or suspensions. A compound of Formula I, as disclosed herein, can be efficacious when administered by suppository administration, among other routes of administration. The most convenient manner of administration is generally oral using a convenient daily dosing regimen which can be adjusted according to the severity of the disease and the patient's response to the medication.

A compound or compounds disclosed herein, as well as their pharmaceutically acceptable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as suspensions, emulsions, or filled capsules for oral use; or in the form of suppositories for rectal or vaginal administration. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the desired dose and pharmacokinetic parameters.

The term “excipient” as used herein is not particularly limited and should be known to a person of skill in the art. The term refers to a compound that is used to prepare a pharmaceutical composition, and is generally safe, non-toxic and neither biologically nor otherwise undesirable, and includes excipients that are acceptable for veterinary use as well as human pharmaceutical use. The compounds disclosed herein can be administered alone but will generally be administered in admixture with one or more suitable pharmaceutical excipients, diluents or carriers selected with regard to the intended route of administration and standard pharmaceutical practice.

The term ‘pharmaceutically acceptable salts’ have been described herein above.

Solid form preparations include powders, tablets, pills, capsules, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs and aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous suspensions can be prepared by dispersing the finely divided active component in water with viscous material, such as natural or synthetic gums, resins, methylcellulose, sodium carboxymethylcellulose, and other well-known suspending agents.

A compound of Formula I, as disclosed herein, may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

A compound of Formula I, as disclosed herein, may be formulated for vaginal administration. Pessaries, tampons, creams, gels, pastes, foams or sprays containing in addition to the active ingredient such carriers should be known to a person of skill in the art, or can be determined, based on the particular application requirements, and as appropriate.

Suitable formulations along with pharmaceutical carriers, diluents and excipients are described in Remington: The Science and Practice of Pharmacy 1995, edited by E. W. Martin, Mack Publishing Company, 19th edition, Easton, Pennsylvania, which is hereby incorporated by reference. A compound of Formula I, as disclosed herein, can also be encapsulated in liposomes, such as those disclosed in U.S. Pat. Nos. 6,180,134, 5,192,549, 5,376,380, 6,060,080, 6,132,763, each of which is incorporated by reference. A skilled formulation scientist may modify the formulations within the teachings of the specification to provide numerous formulations for a particular route of administration without rendering the compositions, as disclosed herein, unstable or compromising their therapeutic activity.

The modification of a compound of Formula I, as disclosed herein, to render them more soluble in water or other vehicle, for example and without limitation, may be easily accomplished by minor modifications (e.g., salt formulation), which are well within the ordinary skill in the art, or can be determined. It is also well within the ordinary skill of the art to modify the route of administration and dosage regimen of a particular compound in order to manage the pharmacokinetics of the present compounds for maximum beneficial effect in patients.

In a fourth aspect, the specification relates to a use of a compound, its stereoisomer, salt, hydrate, solvate, isotope or crystalline form thereof, represented by formula I, for treatment or prophylaxis of a disease:

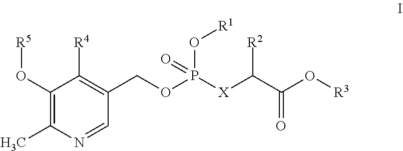

wherein X, R¹, R², R³, R⁴ and R⁵ are as described herein.

In a further aspect, the specification also relates to a use of a compound, its stereoisomer, salt, hydrate, solvate, isotope or crystalline form thereof, represented by formula I (as noted herein), in the preparation of a medicament, for treatment or prophylaxis of a disease

The term “medicament” as used herein is not particularly limited, and should be known to a person of skill in the art. The term relates to a substance used in a method of treatment and/or prophylaxis of a subject in need thereof, wherein the substance includes, but is not limited to, a composition, a formulation, a dosage form, and the like, containing the compound of Formula I. It is contemplated that the compound or the use of the compound represented by Formula I in the manufacture of a medicament for the treatment of any of the conditions disclosed herein.

As noted above, the compound of Formula I, as disclosed herein, can function as prodrugs, and have shown to release the active form pyridoxal 5-phosphate (P5P) both in vivo and in vitro. In addition, these prodrugs have shown to improve the bioavailability of P5P (see dosing data). Further, the compound of Formula I, as disclosed herein, can be used to treat several P5P-related conditions and to play all the roles which P5P is known to play.

Exemplary roles that can be played by the disclosed prodrug compounds (the compound of Formula I, as disclosed herein) include, for example and without limitation, being a cofactor in several enzymatic reactions including but not limited to the metabolism and biosynthesis of histamine, serotonin, and GABA (gama-aminobutyric acid).

In one embodiment, the present specification provides prodrug compounds which can be used to treat epileptic seizures in patients with known PNPO deficiency as well as other clinical or biochemical features associated with impaired vitamin-B6 metabolism. These include, but are not limited to, congenital hypo-phosphatasia, P5P binding protein (PLPBP) deficiency, pyridoxine-dependent epilepsy, hyperprolinaemia type II, and molybdenum cofactor deficiency.

In another embodiment, the present specification provides prodrug compounds which can be used to treat movement disorders including, for example and without limitation, dystonia, homocystinuria (dystonia, parkinsonism), carpal tunnel syndrome, Tourette syndrome, and tardive dyskinesia.

In a further embodiment, the specification provides prodrug compounds which can be used in the treatment of clinical or biochemical issues associated with B6 deficiency including, but not limited to, cardiovascular disease, ischemic heart disease, blood pressure, type 2 diabetes, immunity or chronic inflammation, pneumonia, depression/anxiety, or cancer.

Provided in Table 4 below is a list of potential therapeutic uses of the compound of Formula I, disclosed herein, that are associated with impaired B6 metabolism.

TABLE 4
List of Potential Therapeutic Uses for P5P

| Clinical or Biochemical | |
|---|---|
| feature associated with | |
| Impaired B6 Metabolism | Mechanism |
| B6-responsive seizures | |
| PNPO deficiency | PLP synthesis and recycling |
| Congenital hypo-phosphatasia | PLP import to the brain impaired |
| | due to Alkaline phosphatase |
| | deficiency (TNALP, TNAP, |
| | TNSALP). |
| | Since, TSNALP is GPI anchored |
| | deficiency in GPI anchors |
| | biosynthesis machinery (PIGO, |
| | PIGV) can also result in PLP |
| | deficiency. |
| PLP binding protein (PLPBP) | Abnormal intracellular PLP transport |
| deficiency | and disorders in which metabolites |
| | accumulate that inactive PLP |
| | (PROSC deficiency) |
| Pyridoxine-dependent epilepsy | Metabolite builds up and inactivates |
| | PLP (ALDH7A1 deficiency) |
| Hyperprolinaemia type II | Metabolite builds up and inactivates |
| | PLP (ALDH4A1 deficiency) |
| Molybdenum cofactor | Molybdenum cofactor biosynthesis |
| deficiency | pathway deficiencies (MOSC1, |
| | MOCS2, GPHN) |
| Movement disorders | |
| Aromatic amino acid | Aromatic amino acid decarboxylase |
| decarboxylase deficiency | deficiency |
| (Dystonia) | |
| Homocystinuria (Dystonia, | Cystathione beta synthase (CBS) |
| Parkinsonism) | deficiency |
| Carpal Tunnel Syndrome | B6 treatment may be beneficial |
| Tourette Syndrome | B6 with magnesium may be |
| | beneficial |
| Tardive dyskinesia | Oxidative stress as a result of long- |
| | term antipsychotic medication |
| | may be attenuated by B6 |
| Other drug-induced | Not necessarily any evidence for |
| movement disorders | successful B6 treatment |
| Acute dystonic reaction | |
| Akathisia | |
| Drug-induced parkinsonism | |
| Neuroleptic malignant | |
| syndrome | |
| Withdrawal emergent | |
| dyskinesia | |
| Oculogyric crisis | |
| Other tardive movement | |
| disorders | |
| (Tardive dystonia, Tardive | |
| Chorea, Tardive Tourettism, | |
| Tardive Myoclonus) | |
| Other movement disorders | Not necessarily any evidence for |
| Ataxia | successful B6 treatment |
| Cervical dystonia | |
| Chorea | |
| Dystonia | |
| Functional movement disorder | |
| Huntington's disease | |
| Multiple system atrophy | |
| Myoclonus | |
| Parkinson's disease | |
| Parkinsonism | |
| Progressive supranuclear | |
| palsy | |
| Restless legs syndrome | |
| Tardive dyskinesia | |
| Tremor | |

Provided in Table 5 below is a list of potential therapeutic uses of the compound of Formula I, disclosed herein, that are associated with B6 deficiency.

TABLE 5
List of Potential Therapeutic Uses for P5P

| Clinical or Biochemical | |
|---|---|
| feature associated with | |
| B6 Deficiency | Mechanism |
| Cardiovascular Disease | Potential to reduce the risk of CHD and |
| | nonfatal myocardial infarction |
| | B6 May act at least in part through anti- |
| | inflammatory effects |
| Ischemic Heart Disease | B6 may attenuate intracellular Calcium |
| | overload due to blockade of purinergic |
| | receptors |
| Blood Pressure | B6 may lower blood pressure, the |
| | biochemical reasons remain unresolved |
| | (reduced homocysteine, aldehydes) |
| Type 2 diabetes | Inverse relationship between PLP and the |
| | occurrence of diabetes |
| | It is not entirely clear whether lowered PLP |
| | levels contribute to the development of |
| | diabetes, or whether diabetes lowers PLP |
| | levels |
| Immunity and Chronic | Vitamin B6 plays a key role in the production |
| inflammation | of T lymphocytes and interleukins |
| | There is an inverse relationship between |
| | vitamin B6, and IL-6 and TN-alpha levels in |
| | conditions of chronic inflammation. |
| | The lowest level of PLP found in people with |
| | chronic inflammation, and the highest levels |
| | found in people with low levels of |
| | inflammation. |
| Pneumonia | The anti-inflammatory effect of vitamin B6 |
| | was demonstrated in mice with acute |
| | pneumonia |
| Depression/Anxiety | Low levels of B6 have been associated with |
| | depression, potentially linked to the |
| | biosynthesis of several neurotransmitters. |
| Cancer | A strong inverse relationship between |
| | dietary B6 intake/PLP levels and cancer risk |
| | Likely a result of anti-inflammatory effects |

In a fifth aspect, the specification relates to a method of medical treatment or prophylaxis of a disease, comprising administering, to a subject in need thereof, a therapeutically effective amount of a compound, its stereoisomer, salt, hydrate, solvate, isotope or crystalline form thereof, represented by formula I:

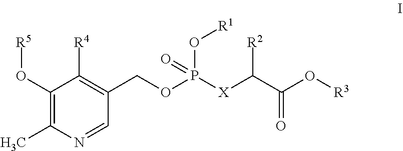

wherein X, R¹, R², R³, R⁴ and R⁵ are as described herein.

It is intended that a subject in need thereof is one that has any condition as disclosed herein.

The term “subject” as used herein is not particularly limited and should be known to a person of skill in the art. The term means a mammal, which includes, but is not limited to, cattle, pigs, sheep, chicken, turkey, buffalo, llama, ostrich, dogs, cats, and humans. In one embodiment, for example and without limitation, the subject is a human.

The term “therapeutically effective amount” as used herein is not particularly limiting and should be known to a person of skill in the art. The term means an amount required to reduce symptoms of the disease in an individual. The dose was adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.1 and about 10 g, including all values in between, such as 0.25, 0.5, 0.75, I5 1.5, 2, 2.5, 3, 3.5, 4, 4.5, 5, 5.5, 6, 6.5, 7, 7.5, 8, 8.5, 9, and 9.5, per day can appropriate. In one embodiment, for example and without limitation, the daily dosage is between about 0.5 and about 7.5 g per day, more preferred 1.5 and about 6.0 g per day. One of ordinary skill in treating diseases described herein will be able, without undue experimentation and in reliance on personal knowledge, experience and the disclosures of this application, to ascertain a therapeutically effective amount of the compounds of Formula I, as disclosed herein, for a given disease and patient.

Therapeutic efficacy can be ascertained from tests of liver function including, but not limited to protein levels such as serum proteins (e.g., albumin, clotting factors, alkaline phosphatase, aminotransferases (e.g., alanine transaminase, aspartate transaminase), 5′-nucleosidase, [gamma]-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism.

### Biological Assay

### Single Ascending Dose Studies

Single ascending dose studies were carried out (Example 97: P5P analog 16i: single ascending dose study) to assess the maximum tolerability of a P5P analog (compound of formula 16i) in rats under 4 different doses under one single administration via Per Os (P.O.—oral) route, and to inform the linearity of pharmacokinetics (PK) with multiple dosing.

In order to isolate the effects of the administered compound of formula 16i on pyridoxine (PL) and P5P (PLP) levels, the basal levels of both PL and P5P were subtracted from the measured concentrations. Baseline subtraction was accomplished by subtracting pre-dose concentrations of either PL or P5P from the subsequent values in the time course.

A clear dose-dependent increase in plasma PL and P5P concentrations were observed confirming the effectiveness of compound of formula 16i. Increasing the dose of the compound of formula 16i resulted in more P5P in the plasma.

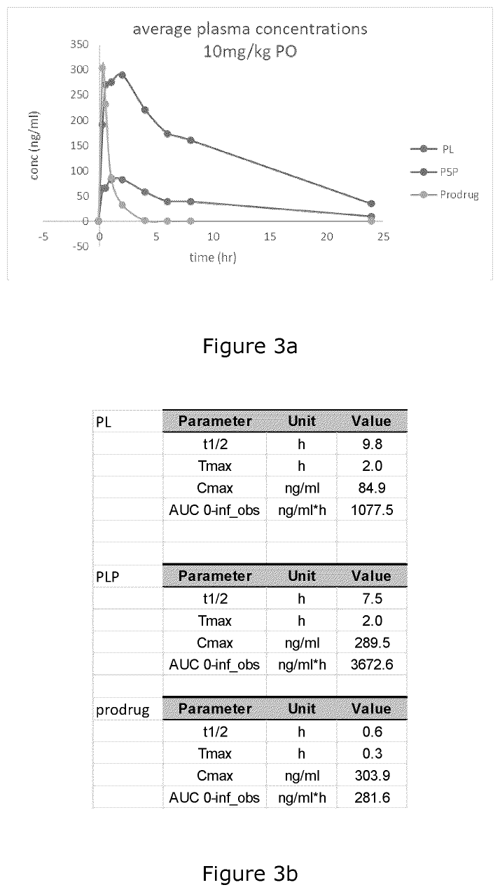

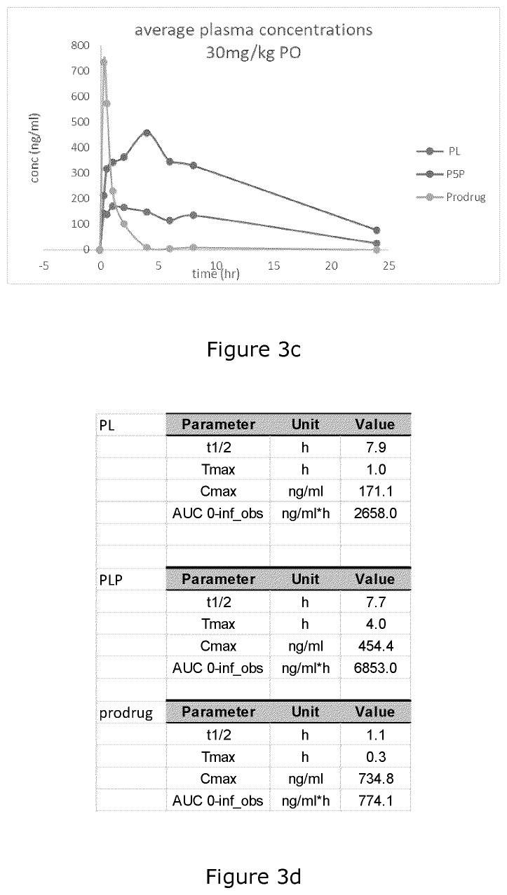

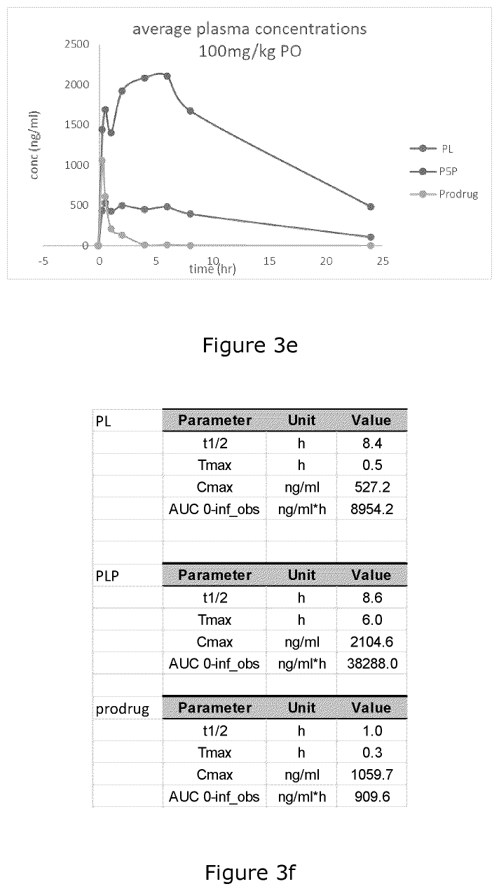

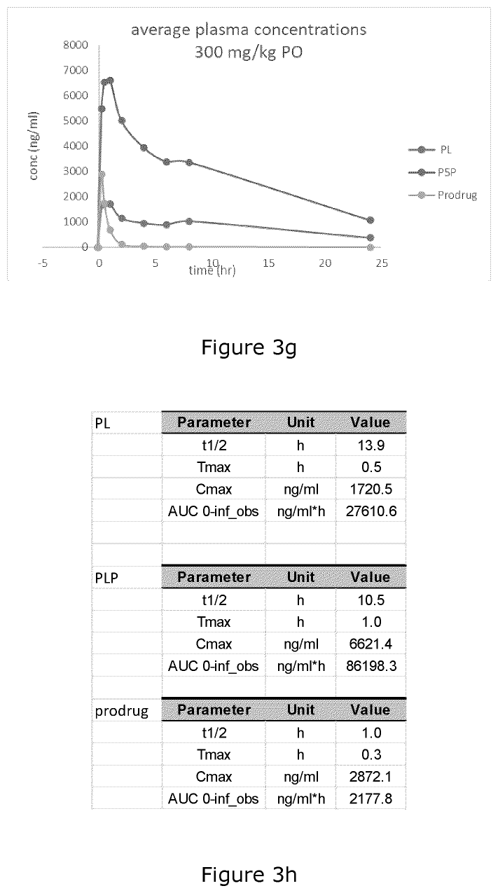

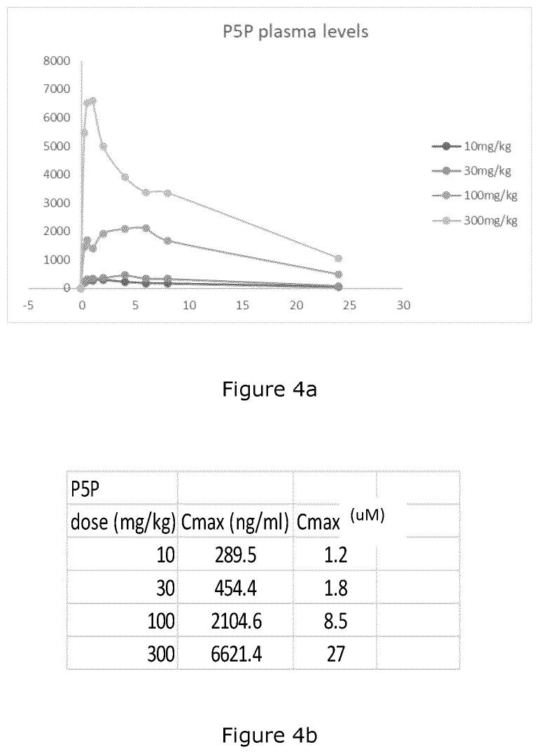

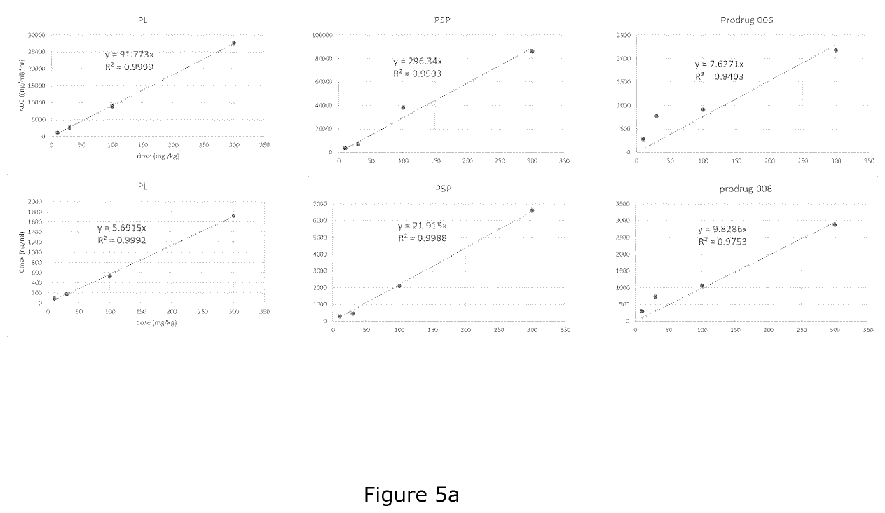

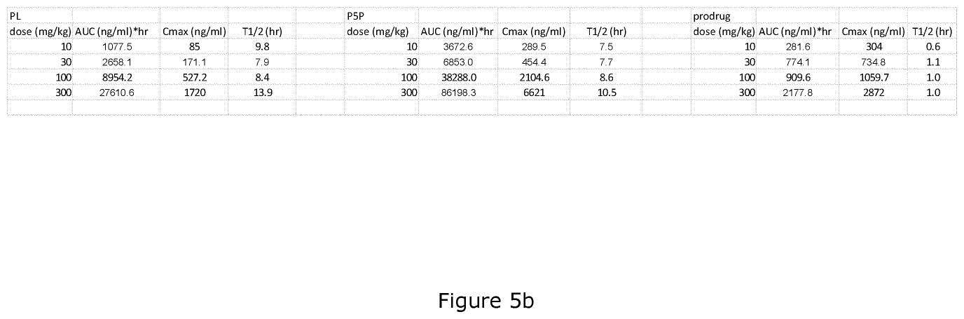

Based on the results obtained (see FIGS. 3-5), the effectiveness of the compound of formula 16i was confirmed as there was a clear dose-dependent increase in plasma PL and PLP concentrations. In this study the MTD (maximum tolerable dose) was not determined as the animals in the highest dose group (300 mg/kg) did not show any signs of toxicity (despite high plasma levels of PL and PLP). The compound of formula 16i showed dose linearity up to 300 mg/kg for plasma PL and PLP. Linearity was seen for both peak concentration (Cmax) and exposure (AUC). This allows repeat dose plasma levels to be predicted.

In addition, the compound of formula 16i was safe over 24 hours at the tested doses given no clinical signs were observed. An increase in the T_{1/2} was noted for PL and PLP as dose was increased. This may indicate that clearance mechanisms are slowing/saturating with increasing dose and should be monitored. Also possible that a second source is adding PL and PLP to the plasma. Lack of dose linearity with the prodrug may be due to instability of prodrug during analysis.

### Pharmacokinetic Study 1

Pharmacokinetic study 1 was carried out (see Example 98: P5P analog 16i: Pharmacokinetic study 1) to assess if P5P analog 16i was able to be converted to P5P and increase plasma P5P, to assess if P5P analog 16i was orally available, and to compare the ability of PO P5P analog 16i and PO P5P to increase plasma P5P.

In order to isolate the effects of the administered P5P analog 16i on pyridoxine (PL) and P5P (PLP) levels, the basal levels of both PL and P5P were subtracted from the measured concentrations. Baseline subtraction was accomplished by subtracting pre-dose concentrations of either PL or P5P from the subsequent values in the time course.

When dosed IV, P5P analog 16i was converted to P5P. The half life of this P5P was greater that when P5P was dosed IV. Further, IV dosing of the P5P analog 16i was seen to produce elevated levels of P5P in plasma confirming that the P5P analog 16i was being converted to the desired product.

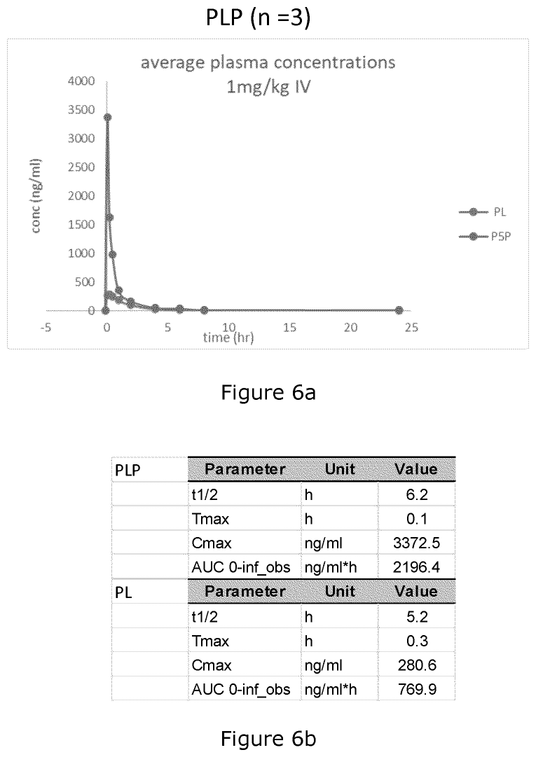

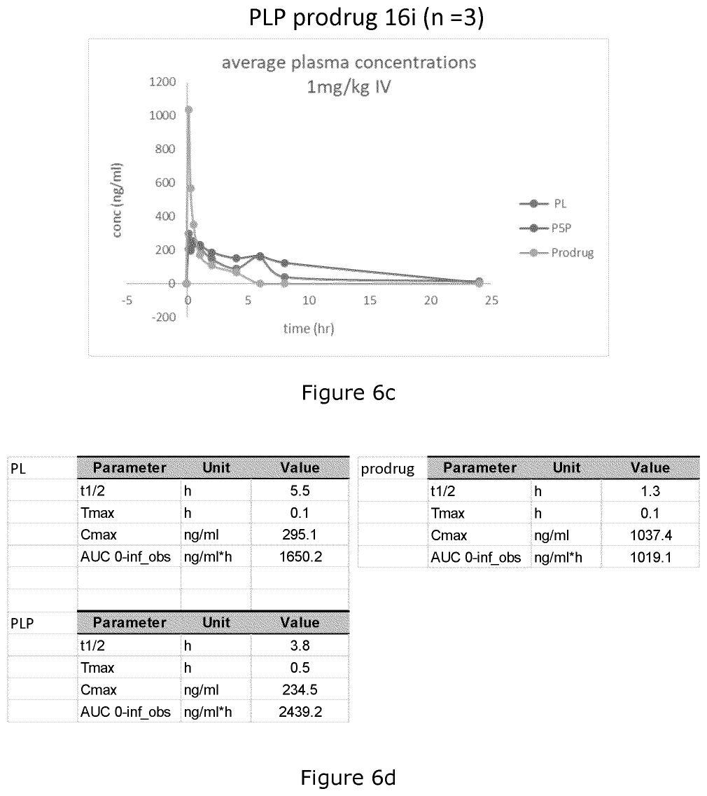

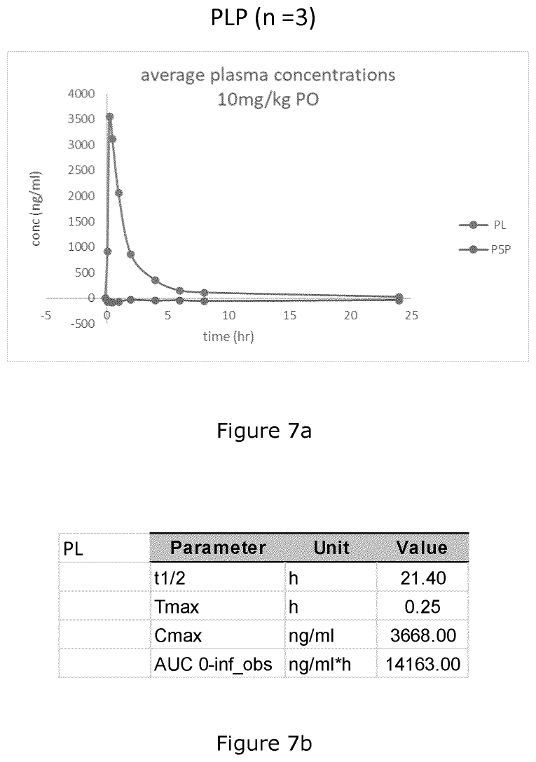

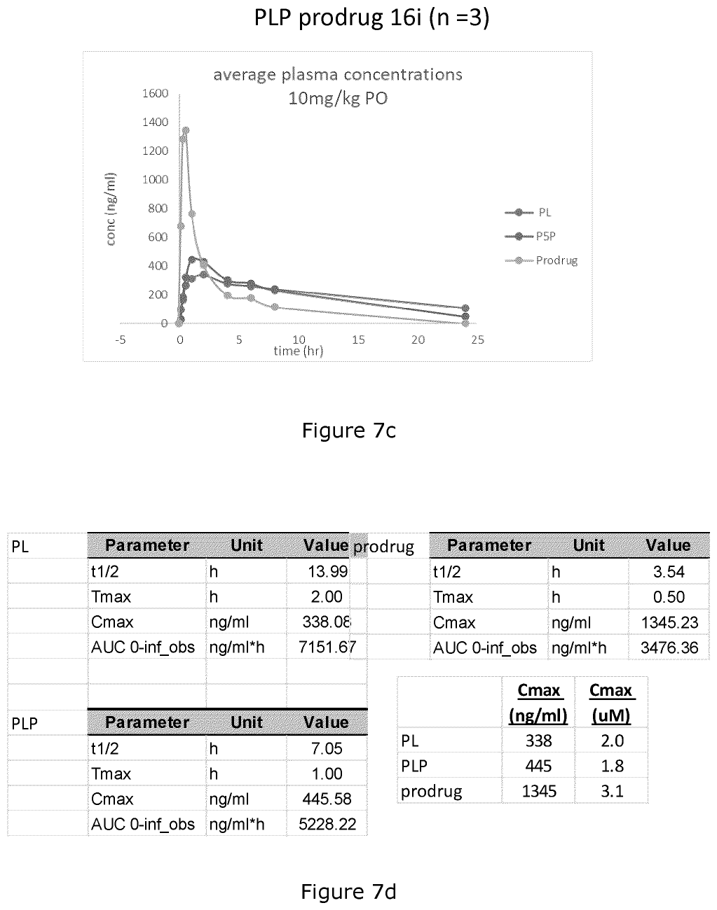

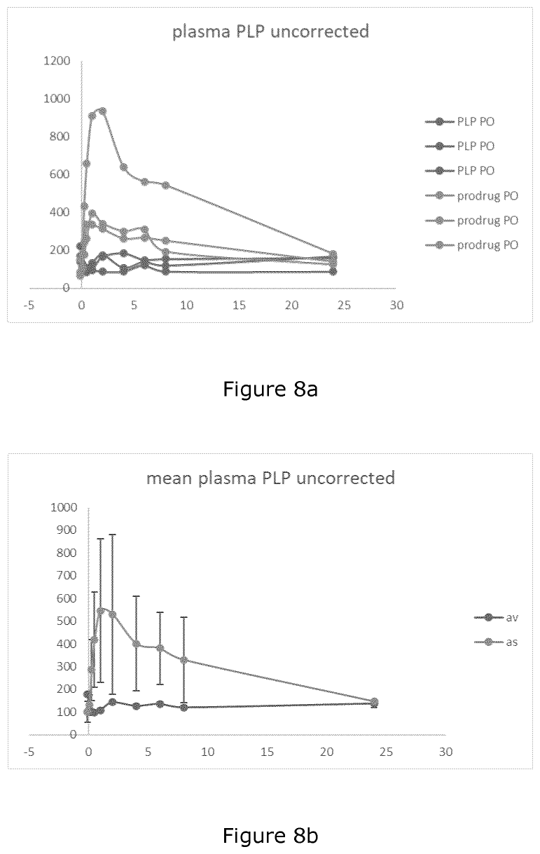

Based on the results obtained (see FIGS. 6-8), when dosed orally, P5P analog 16i was able to cross the gastrointestinal wall (orally bioavailable) and was detectable in the circulatory system. After oral dosing of P5P analog 16i, elevated plasma levels of P5P were detected. This elevated P5P had a half-life of approximately 7 hours. Oral dosing with P5P itself resulted in no detectable P5P in the plasma over a 24-hour period. This indicates that oral bioavailability of P5P is 0%. The calculated oral bioavailability of P5P from dosing of P5P analog 16i was 43%.

### Pharmacokinetic Study 2

Pharmacokinetic study 2 was carried out (Example 99: P5P analog 16i: Pharmacokinetic study 2) to assess the effect of the P5P analog 16v compared to P5P analog 16i.

In order to isolate the effects of the administered P5P analogs 16i and 16v on pyridoxine (PL) and P5P levels, the basal levels of both PL and P5P were subtracted from the measured concentrations. Baseline subtraction was accomplished by subtracting pre-dose concentrations of either PL or P5P from the subsequent values in the time course.

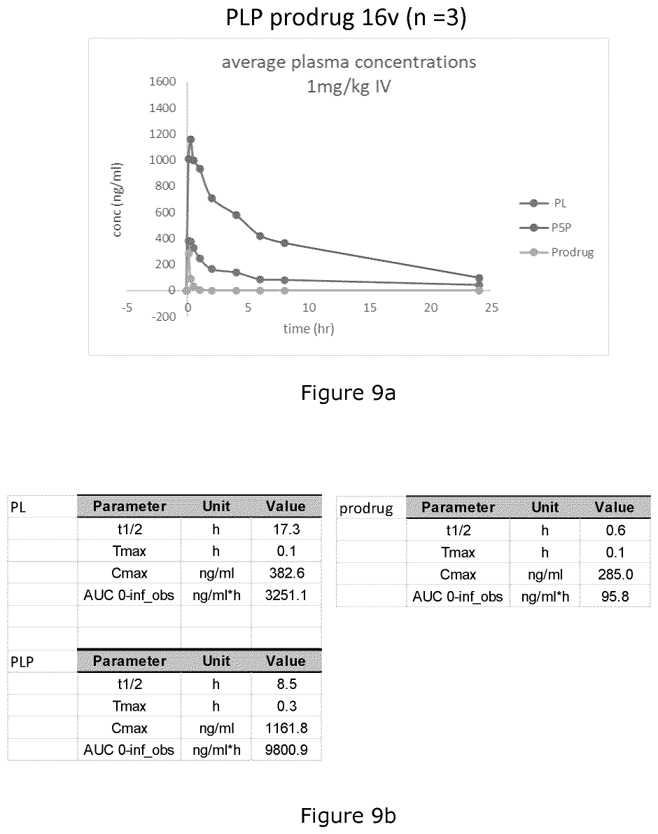

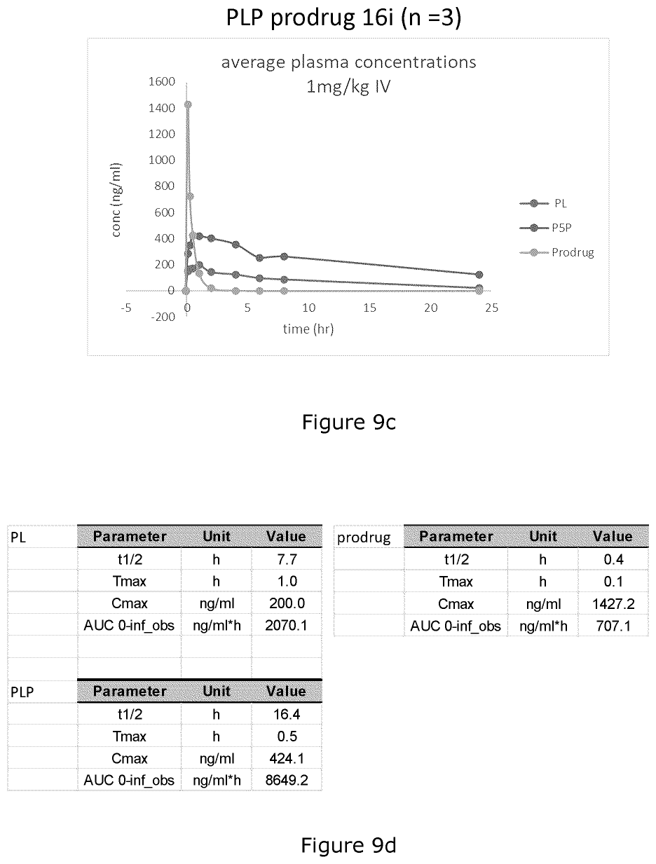

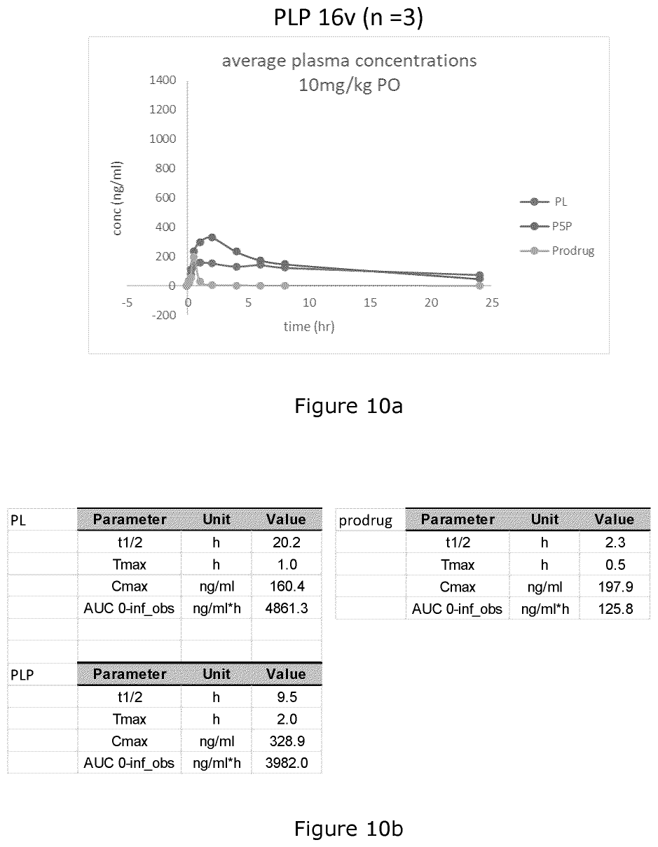

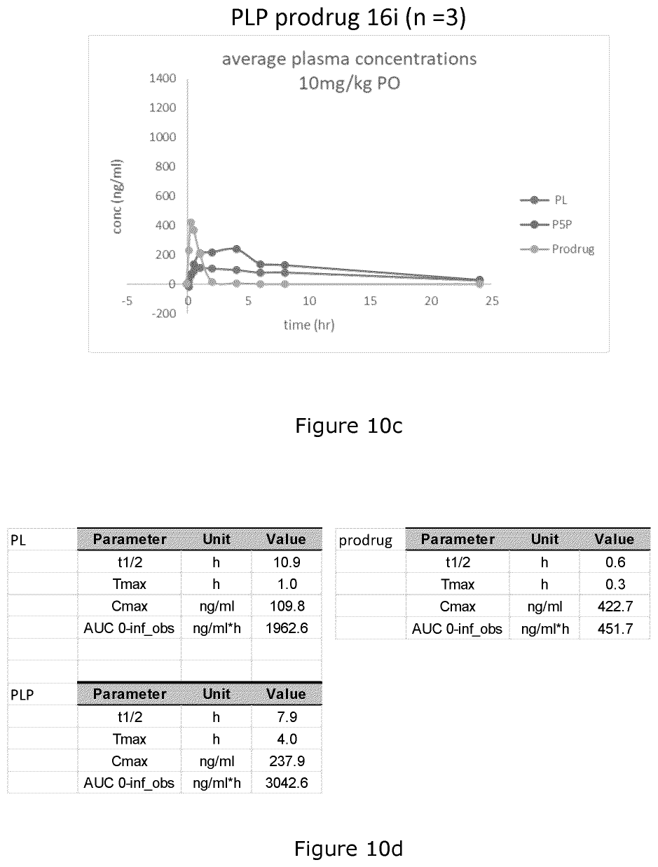

Based on the results obtained (see FIGS. 9-10), when dosed IV, P5P analog 16v was converted to P5P very efficiently, more so than with P5P analog 16i. When dosed orally, P5P analog 16v was able to cross the gastrointestinal wall (orally bioavailable) and was detectable in the circulatory system. After oral dosing of P5P analog 16v, elevated plasma levels of P5P were detected and showed slightly greater amounts compared to P5P analog 16i. This difference was attributable to a single rat. P5P analog 16v when dosed orally showed no clear advantage over P5P analog 16i.

### Pharmacokinetic Study 3

Pharmacokinetic study 2 was carried out (Example 100: P5P analog 16vi and compound II.I.: Pharmacokinetic study 3) to assess the effect of a P5P analog not encompassed by the compound of Formula I (Compound II.I.), amino acid modification (P5P analog 16vi) and compounds of formula 16iii, 16i and a racemic mixture of 16i.

In order to isolate the effects of the administered Compound II.I. and P5P analog 16vi on pyridoxine (PL) and P5P levels, the basal levels of both PL and P5P were subtracted from the measured concentrations. Baseline subtraction was accomplished by subtracting pre-dose concentrations of either PL or P5P from the subsequent values in the time course.

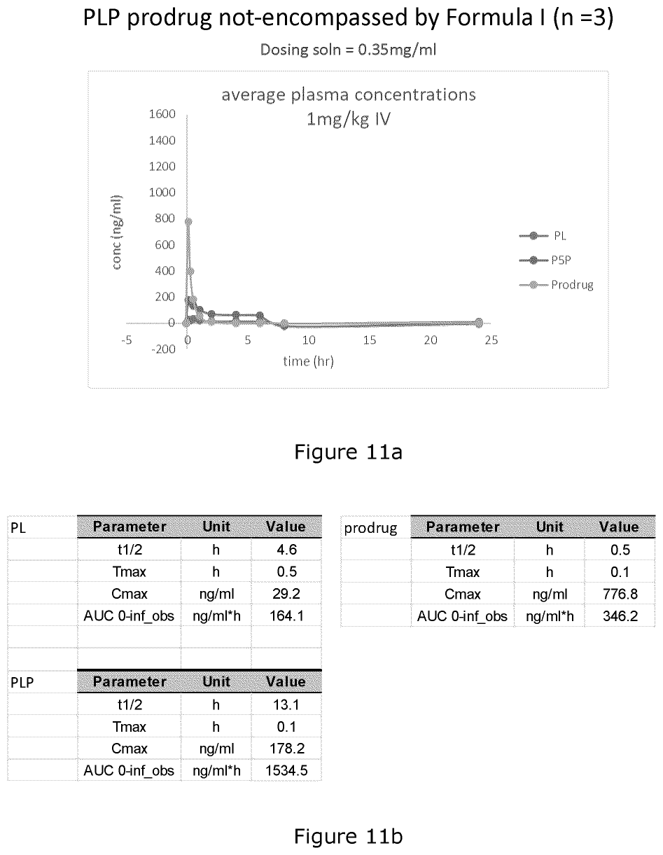

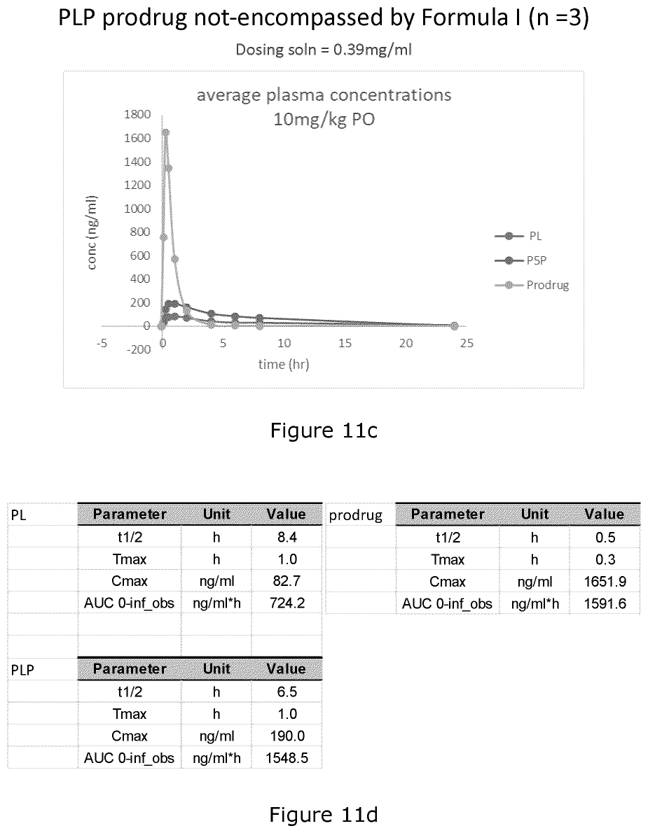

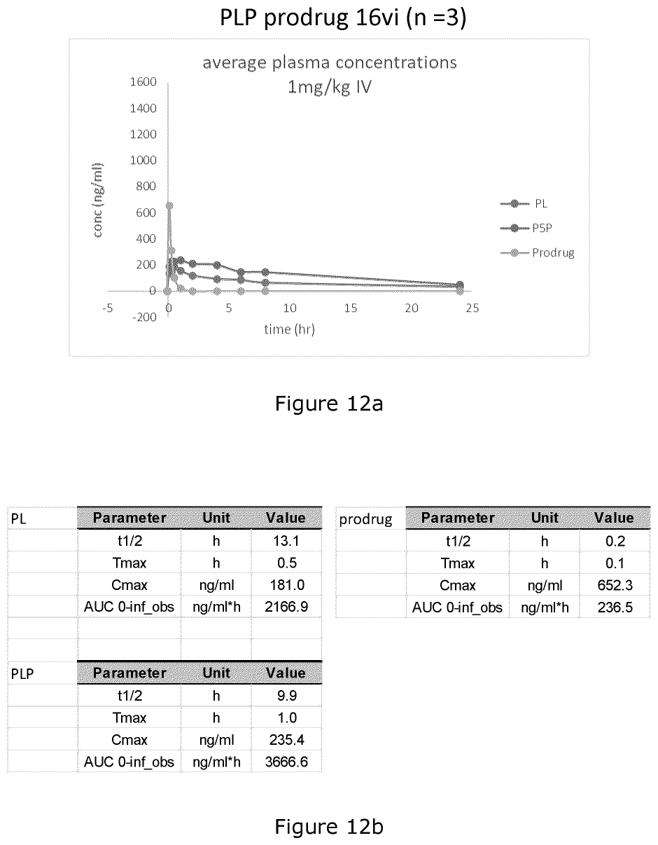

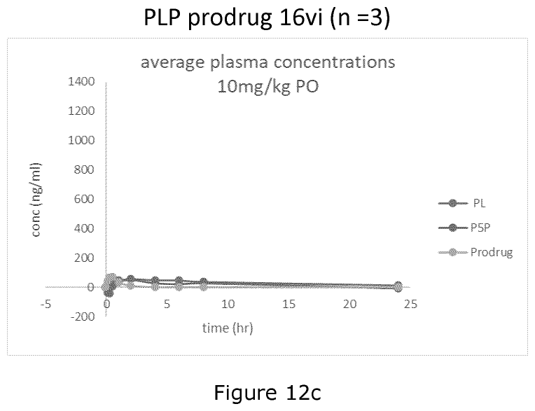

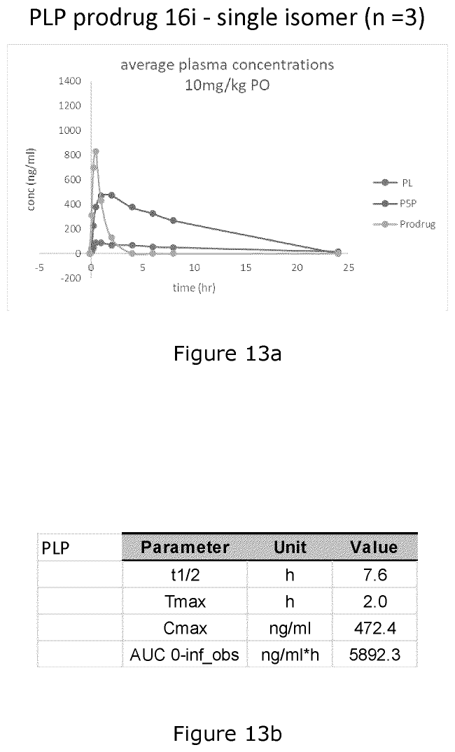

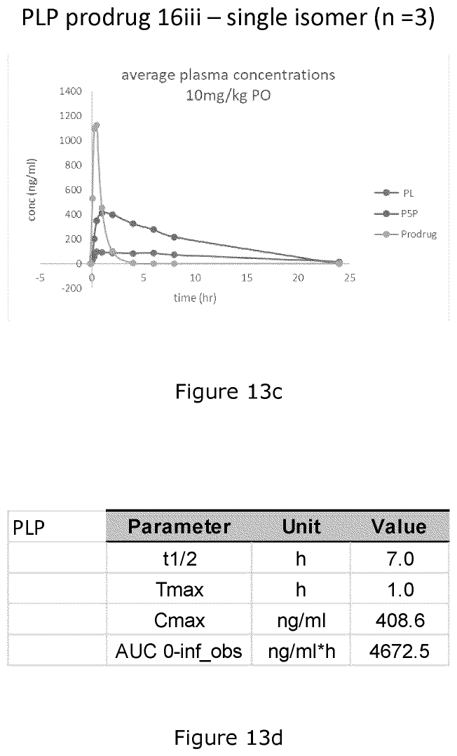

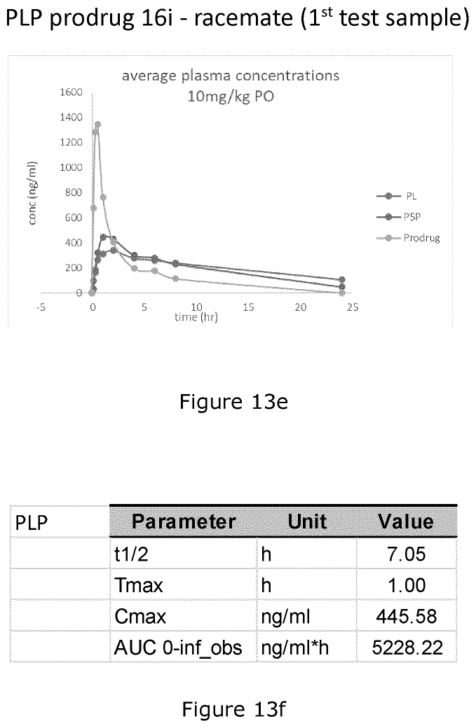

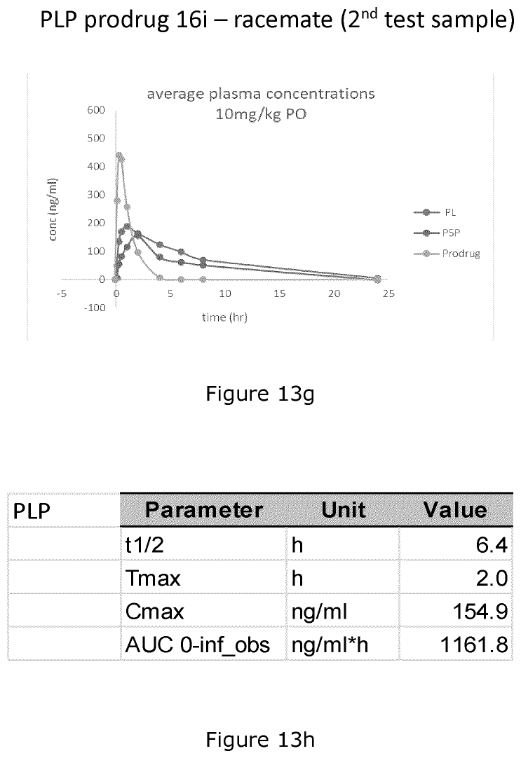

Based on the results obtained (see FIGS. 11-13), when dosed orally, Compound II.I. was able to cross the gastrointestinal wall (orally bioavailable) and was detectable in the circulatory system. After oral dosing of Compound II.I., elevated plasma levels of P5P were detected and showed similar amounts compared to P5P analog 16i. P5P analog 16vi did not demonstrate significant oral bioavailability.

### Pharmacokinetic Study 4

The goal of the study was to compare the pharmacokinetic (PK) profiles of the S-isomer (16i) and the R-isomer (16iii) at the phosphorus center, and to assess the effect of using the D-amino acid (16i with S_{P} and R_{P} isomers) on PK profile.

In order to isolate the effects of the administered agents on PL and PLP levels, the basal levels of both PL and PLP subtracted from the measured concentrations. Baseline subtraction was accomplished by subtracting pre-dose concentrations of either PL or PLP from the subsequent values in the time course.

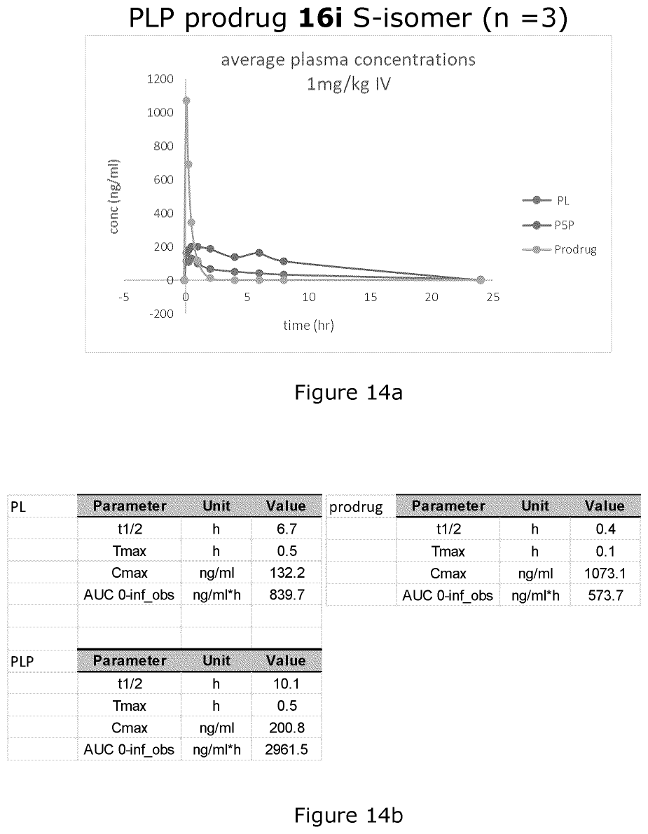

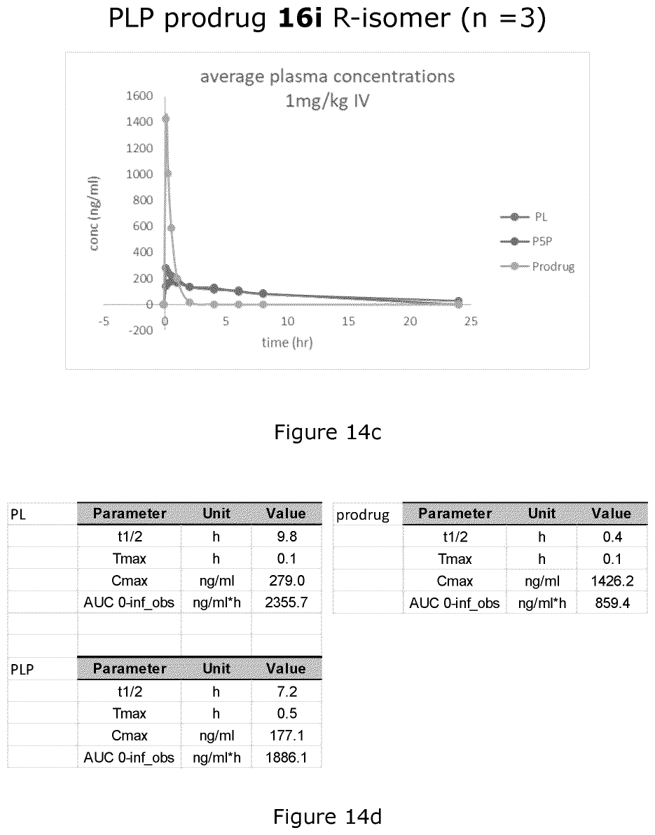

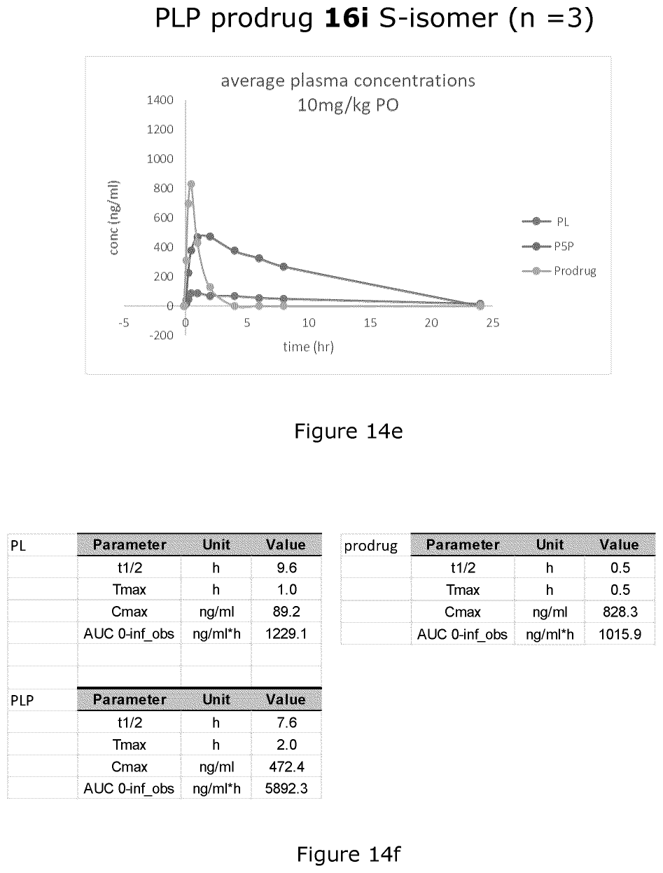

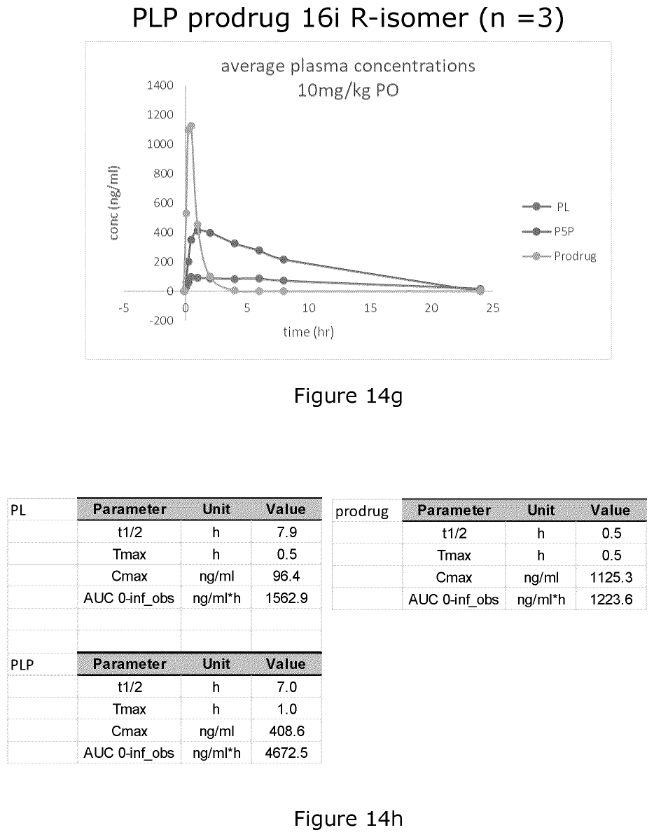

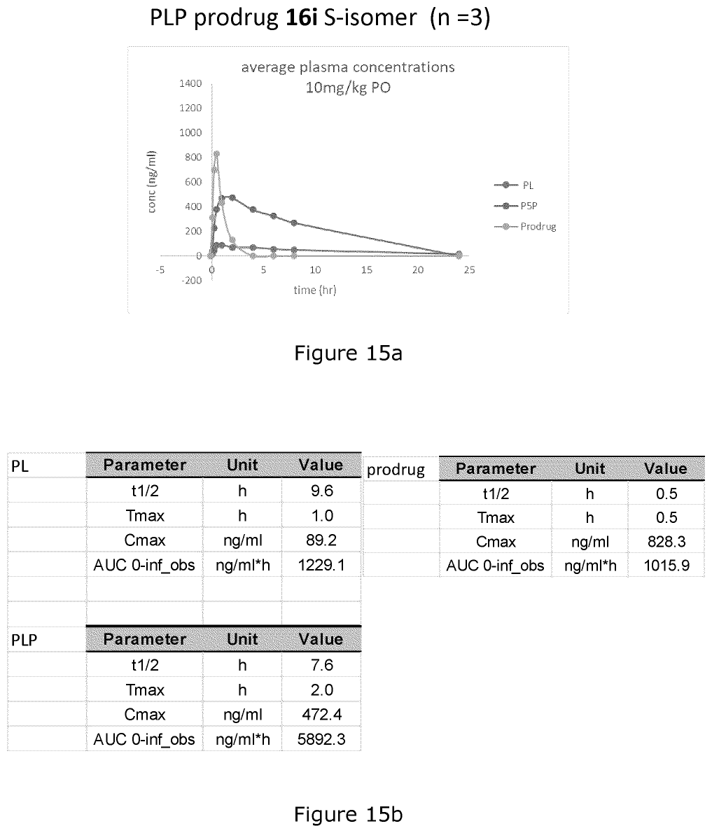

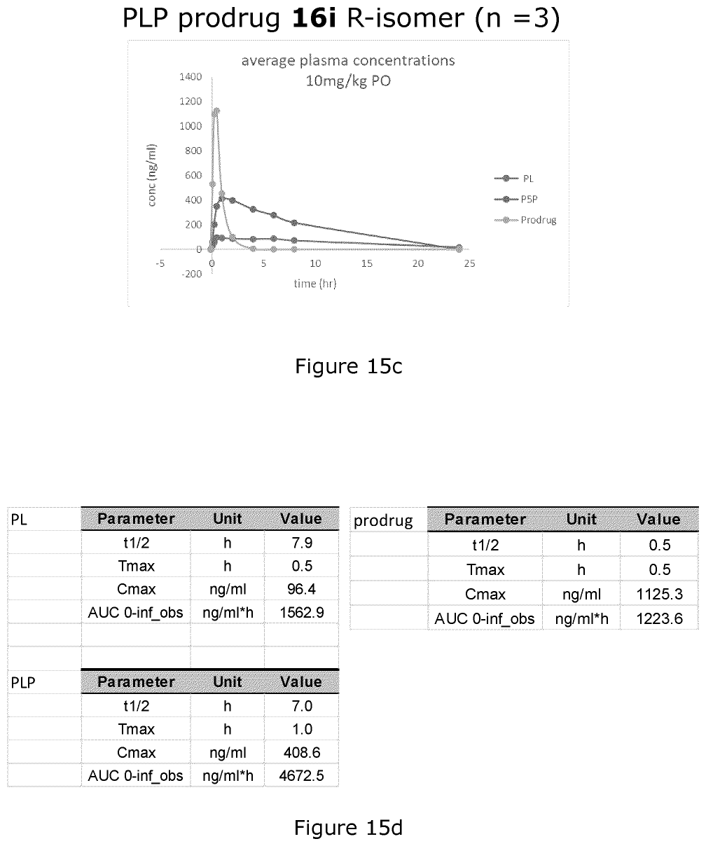

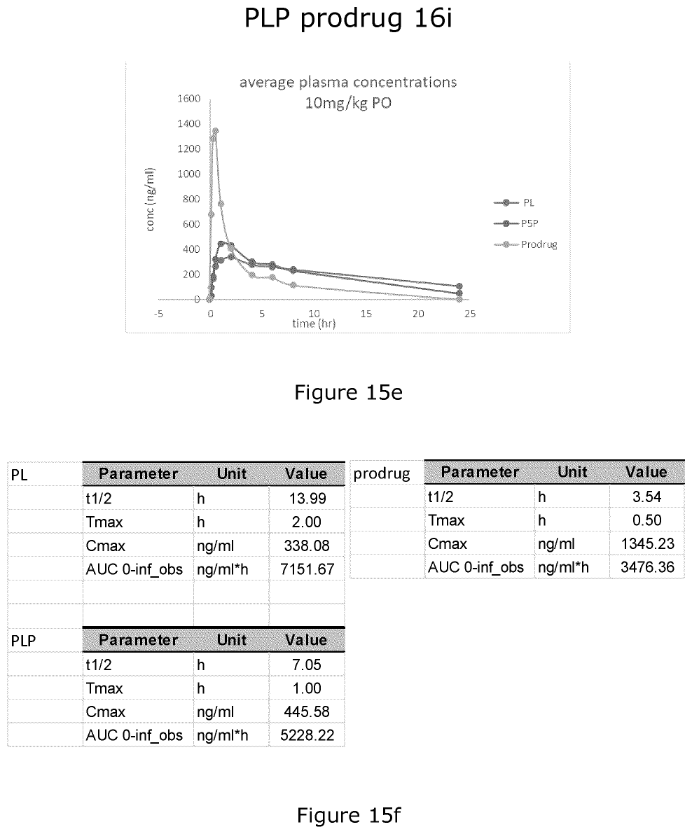

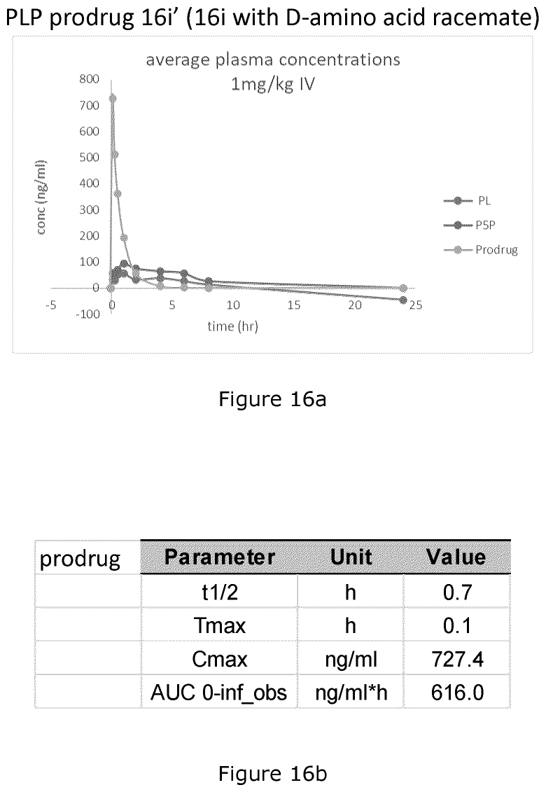

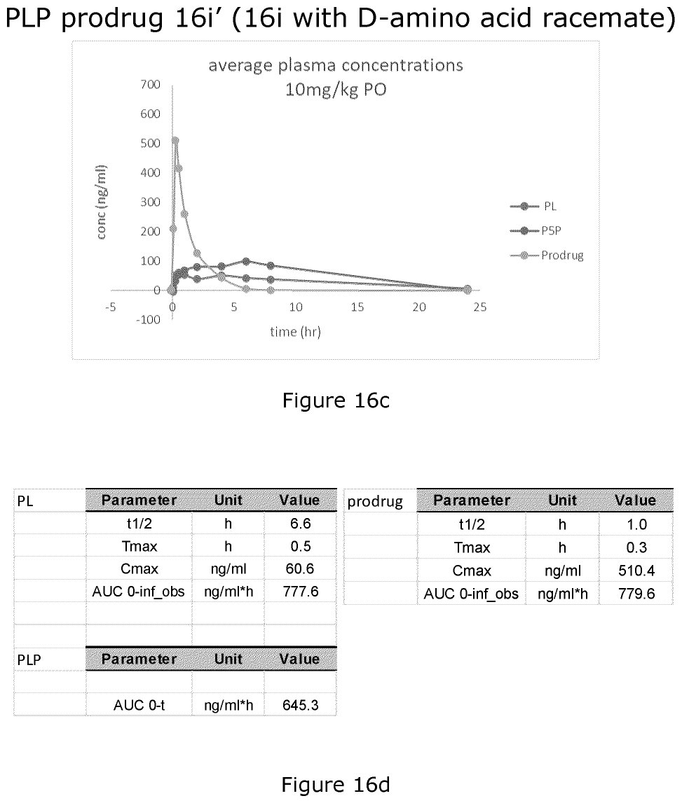

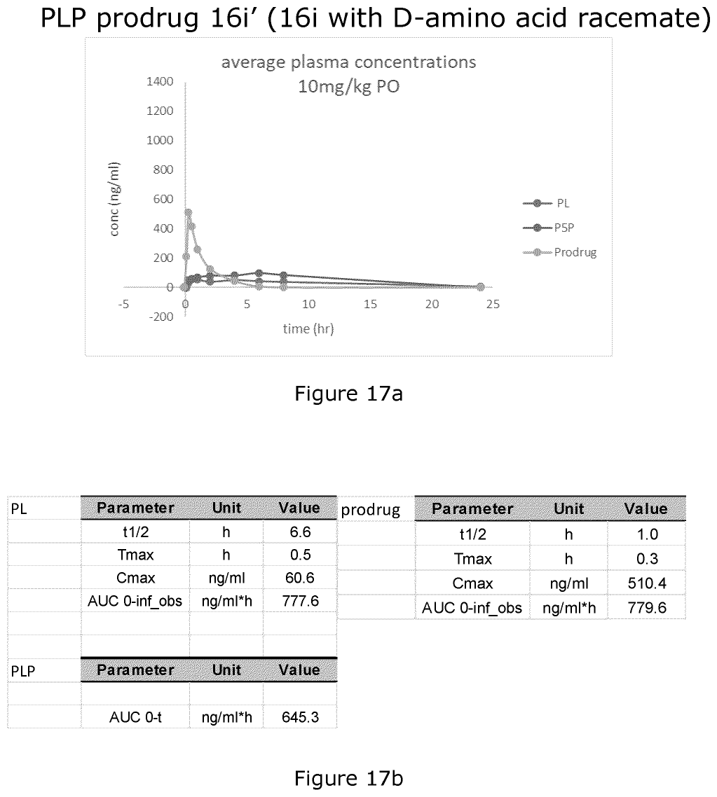

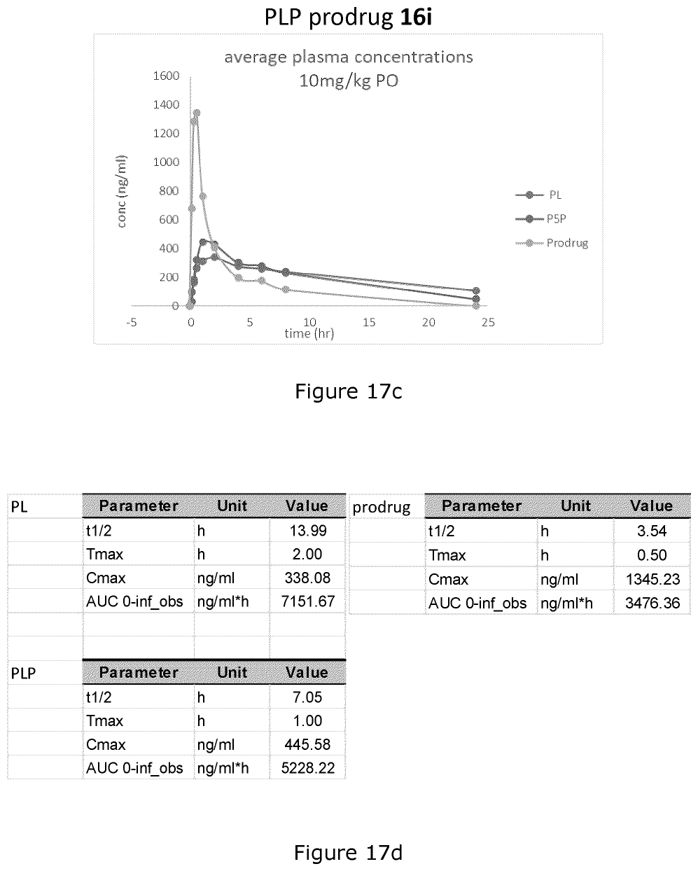

Based on FIGS. 14-17, 16i (S_{P} isomer) IV and PO data in the current study suggest that this isomer is readily converted to PLP in the plasma, and is consistent with the 16i (racemate) data. Both the IV and PO data suggest there is little difference between the R_{P} and S_{P} isomers with respect to amount of PLP released into the plasma. In comparing the racemic mixtures containing the D-amino acid 16i′ with the L-amino acid 16i, there is reduced PLP exposure with the D-amino acid, and the use of the D-amino acid appears to prolong the PLP half-life by creating a second increase in plasma PLP (possibly delayed release from the liver).

### EXAMPLES

The following examples are illustrative and non-limiting and represent specific embodiments of the present specification.

### General Methods

¹H NMR spectra were recorded on a Bruker Avance 300 NMR spectrometer operating at 299.992 MHz using the solvent resonances as secondary standards. Phenyl dichlorophosphate, H-Ala-OiPr hydrochloride, 4-nitrophenol and other reagents used herein were purchased from Sigma Aldrich or Combi Blocks and were used as received. N,N-diisopropylethylamine, trimethylamine, tetrahydrofuran, acetonitrile, dichloromethane, trifluoroacetic acid, diethyl ether, diisopropyl ether, acetone, hexanes, ethyl acetate, and methanol were purchased from Sigma Aldrich and used without further drying. All reactions were carried out under a nitrogen or Argon atmosphere. Compounds were visualized/located by spraying the TLC plate with Ninyhydrin solution, KMnO₄ solution, or a solution of 2% ceric ammonium sulfate in 0.5 M H₂SO₄ followed by heating on a hot plate until color developed.

### Example 1: Intermediate 5: R₁=Phenyl, R₂=Methyl with S Stereochemistry, R₃=Isopropyl. X═H. (2S)-Isopropyl 2-((4-Nitrophenoxy)(Phenoxy)Phosphorylamino)Propanoate

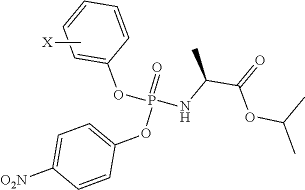

Phenyl phosphorodichloridate (1 equiv.) and (S)-Isopropyl 2-aminopropanoate hydrochloride (1 equiv.) were suspended in dry dichloromethane and the mixture was cooled to −78° C. Triethylamine (2.1 equiv.) was slowly added under argon. After the addition, dry ice/acetone bath was removed. When ³¹P NMR confirmed the complete consumption of the dichloridate and the formation of a new species (1-2 hrs), the reaction mixture was cooled to 0° C. and 4-nitrophenol (0.9-0.95 equiv.) was added in one portion followed by the addition of trimethylamine (2.1 equiv.). The mixture was allowed to warm to RT (2-3 hrs) when TLC confirmed the formation of a new product. Diethyl ether was added and most triethyl ammonium salts were filtered off. The filtrate was concentrated and purification using biotage (hex/EtOAc) gave a pure product as a syrup (80-90% yield).

### Example 2: Resolution of 5 (R₁=Phenyl, R₂=Methyl with S Stereochemistry, R₃=Isopropyl)

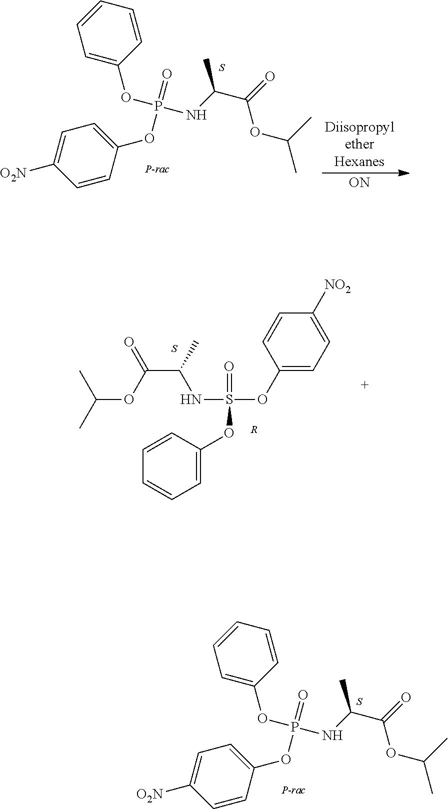

A typical mixture of 5 (P-rac, S) made up of roughly equimolar amounts of the two diastereomers was dissolved in a minimum amount of diisopropyl ether to give a clear solution. Hexanes was slowly added until a slight cloudiness persisted. After stirring the solution slowly overnight, a colorless precipitate (11% of the original mixture) was filtered off and dried. This is P—R, S diastereomer. The filtrate was concentrated and the process was repeated to obtain more of the solid P—R, S diastereomer.

### Synthesis of Pyridoxamine Prodrugs

### Example 3: (2S)-Isopropyl 2-(((4-(aminomethyl)-5-hydroxy-6-methylpyridin-3-yl)methoxy)(phenoxy)phosphorylamino)propanoate (14)

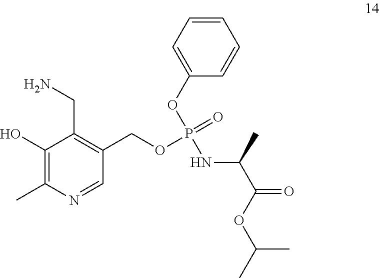 [SMILES: Cc1ncc(COP(=O)(N[C@@H](C)C(=O)OC(C)C)Oc2ccccc2)c(CN)c1O]

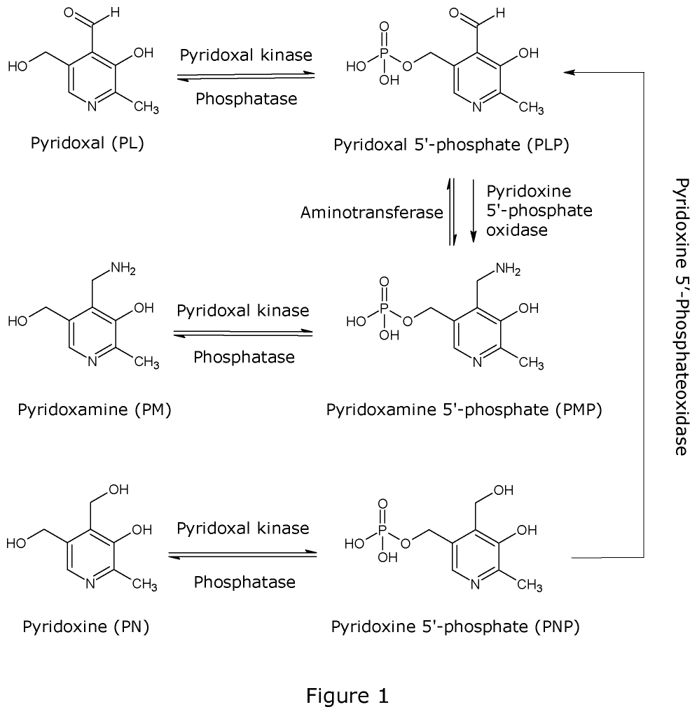

Compound 14 was prepared using the procedure in general scheme 2 above. Specifically, Tert-butyl(3-(tert-butoxycarbonyloxy)-5-(hydroxymethyl)-2-methylpyridin-4-yl)methylcarbamate 11 (1 equiv.), intermediate 5 (P-rac, S) (1.2 equiv.) and MgCl₂ (1 equiv.) were suspended in dry acetonitrile and heated to 50° C. for 10 min under argon. DIPEA (2.5 equiv.) was then added in one portion and heating and stirring at 50° C. were continued until TLC confirmed the completion of the reaction (20 min. to 1 hr). Purification using biotage (hex/EtOAc) gave intermediate 12. Treatment of 12 with TFA/DCM furnished 14 as a TFA salt (typically >80% over two steps). ¹H NMR (299.992 MHz, methanol-d₄) δ 1.17-1.28 (m, 6H, 2CH₃), 1.31-1.41 (m, 3H, CH₃), 2.58-2.65 (br, 3H, ArCH₃), 3.85-4.02 (m, 1H, CH), 4.17-4.35 (m, 2H, CH₂), 4.94-5.08 (m, 1H, CH), 5.25-5.42 (m, 2H, CH₂), 7.12-7.44 (m, 5H, ArH), 8.07-8.18 (m, 1H, ArH). The pyridoxamine prodrugs in FIG. 1 were all synthesized following this procedure.

### Characterization Data for Selected Pyridoxamine Prodrugs

### Example 4: (2S)-2-Ethylbutyl 2-(((4-(aminomethyl)-5-hydroxy-6-methylpyridin-3-yl)methoxy)(phenoxy)phosphorylamino)propanoate (14i)

¹H NMR (299.992 MHz, methanol-d₄) δ 0.83-0.99 (m, 6H, 2CH₃), 1.29-1.69 (m, 8H, 2CH₂, CH₃, CH), 2.66-2.82 (br, 3H, ArCH₃), 3.94-4.18 (m, 3H, CH₂, CH), 4.29-4.44 (m, 2H, CH₂), 5.34-5.53 (m, 2H, CH₂), 7.17-7.45 (m, 5H, ArH), 8.23-8.36 (m, 1H, ArH).

### Example 5: Isopropyl 2-(((4-(aminomethyl)-5-hydroxy-6-methylpyridin-3-yl)methoxy)(phenoxy)phosphorylamino)acetate (14ii)

¹H NMR (299.992 MHz, methanol-d₄) δ 1.08-1.16 (m, 6H, 2CH₃), 2.58 (s, 3H, CH₃), 3.52-3.71 (m, 3H, CH₂, CH), 4.24 (s, 2H, CH₂), 4.88-5.00 (m, 1H, CH), 5.26-5.37 (m, 2H, CH₂), 7.03-7.32 (m, 5H, ArH), 8.17 (s, 1H, ArH).

### Example 6: (2S)-Benzyl 2-(((4-(aminomethyl)-5-hydroxy-6-methylpyridin-3-yl)methoxy)(phenoxy)phosphorylamino)propanoate (14iii)

¹H NMR (299.992 MHz, methanol-d₄) δ 0.041-0.085 (m, 3H, CH₃), 1.25-1.40 (m, 3H, ArCH₃), 2.59-2.78 (m, 1H, CH), 2.85-3.03 (m, 2H, CH₂), 3.69-3.83 (m, 2H, CH₂), 3.91-4.01 (m, 2H, CH₂), 5.69-6.05 (m, 10H, ArH), 6.79-6.95 (m, 1H, ArH).

### Example 7: (2S)-Isopropyl 2-(((4-(aminomethyl)-5-hydroxy-6-methylpyridin-3-yl)methoxy)

### (5,6,7,8-tetrahydronaphthalen-1-yloxy)phosphorylamino)propanoate (14iv)

¹H NMR (299.992 MHz, methanol-d₄) δ1.21-1.29 (m, 6H, 2CH₃), 1.37-1.43 (m, 3H, CH₃), 1.72-1.87 (m, 4H, 2CH₂), 2.61-2.68 (m, 3H, CH₃), 2.68-2.84 (m, 4H, 2CH₂), 3.87-4.03 (m, 1H, CH), 4.20-4.33 (m, 2H, CH₂), 4.96-5.07 (m, 1H, NH), 5.25-5.46 (m, 2H, CH₂), 6.88-6.98 (m, 1H, ArH), 6.99-7.09 (m, 2H, ArH), 8.09-8.22 (m, 1H, ArH).

### Example 8: (2S)-Isopropyl 2-(((4-(aminomethyl)-5-hydroxy-6-methylpyridin-3-yl)methoxy)(4-chlorophenoxy)phosphorylamino)propanoate (14v)

¹H NMR (299.992 MHz, methanol-d₄) δ1.07-1.16 (m, 6H, 2CH₃), 1.20-1.30 (m, 3H, CH₃), 2.53-2.60 (m, 3H, CH₃), 3.75-3.88 (m, 1H, CH), 4.17-4.28 (m, 2H, CH₂), 4.82-4.94 (m, 1H, CH), 5.18-5.34 (m, 2H, CH₂), 7.03-7.15 (m, 2H, ArH), 7.21-7.32 (m, 2H, ArH), 8.06-8.21 (m, 1H, ArH).

### Example 9: (2S)-Isopropyl 2-(((4-(aminomethyl)-5-(dimethylcarbamoyloxy)-6-methylpyridin-3-yl)methoxy)(phenoxy)phosphorylamino)propanoate (14vi)

¹H NMR (299.992 MHz, chloroform-d) δ1.20-1.28 (m, 6H, CH₃), 1.38-1.43 (m, 3H, CH₃), 2.62-2.64 (m, 3H, ArCH₃), 3.12-3.14 (m, 3H, NCH₃), 3.27-3.31 (m, 1H, NH), 3.88-4.07 (m, 1H, CH), 4.35-4.49 (m, 2H, CH), 4.94-5.06 (m, 2H, CH₂), 5.38-5.51 (m, 2H, CH₂), 7.16-7.43 (m, 6H, ArH), 8.59-8.61 (m, 1H, ArH).

### Example 10: (2S)-p-Tolyl 2-(((4-(aminomethyl)-5-hydroxy-6-methylpyridin-3-yl)methoxy)(phenoxy)phosphorylamino)propanoate (14vii)

¹H NMR (299.992 MHz, methanol-d₄) δ 1.56-1.48 (m, 3H, CH₃), 2.33 (s, 3H, ArCH₃), 2.65 (s, 3H, ArCH₃), 4.38-4.16 (m, 3H, CH, CH₂), 5.50-5.33 (m, 2H, CH₂), 6.95-6.81 (m, 2H, ArH), 7.43-7.10 (m, 7H, ArH), 8.29-8.17 (m, 1H, ArH).

### Example 11: Pyridoxamine Prodrug 14 with Deuterated Methyl Groups of the Isopropyl Group (14viii)

¹H NMR (299.992 MHz, methanol-d₄) δ 1.26-1.47 (m, 3H, CH₃), 2.69 (s, 3H, ArCH₃), 3.16-3.29 (m, 1H, CH), 3.86-4.03 (m, 1H, CH), 4.42-4.49 (m, 2H, CH₂), 4.67-4.84 (m, 1H, NH), 5.30-5.51 (m, 2H, CH₂), 7.10-7.27 (m, 3H, ArH), 7.29-7.44 (m, 2H, ArH), 8.15-8.37 (m, 1H, ArH).

### Example 12: (2S)-Isopropyl 2-(((4-(aminomethyl)-5-hydroxy-6-methylpyridin-3-yl)methoxy)(naphthalen-1-yloxy)phosphorylamino)propanoate (14ix)

¹H NMR (299.992 MHz, methanol-d₄) δ1.18-1.23 (m, 6H, CH₃), 1.35-1.41 (m, 3H, CH₃), 2.60-2.62 (m, 3H, ArCH₃), 3.30-3.32 (m, 1H, NH), 4.00-4.19 (m, 1H, CH), 5.29-5.40 (m, 2H, CH), 7.37-7.42 (m, 2H, ArH), 7.51-7.57 (m, 2H, ArH), 7.68-7.73 (m, 1H, ArH), 7.85-7.89 (m, 1H, ArH), 8.04-8.09 (m, 2H, ArH).

### Example 13: (2S)-Cyclopentyl 2-(((4-(aminomethyl)-5-hydroxy-6-methylpyridin-3-yl)methoxy)(phenoxy)phosphorylamino)propanoate (14x)

¹H NMR (299.992 MHz, methanol-d₄) δ1.36-1.26 (m, 3H, CH₃), 1.91-1.55 (m, 8H, 4CH₂), 2.69-2.59 (m, 3H, ArCH₃), 3.98-3.84 (m, 1H, CH), 4.35-4.25 (m, 2H, CH₂), 5.20-5.08 (m, 1H, CH), 5.43-5.28 (m, 2H, CH₂), 7.26-7.15 (m, 3H, ArH), 7.41-7.31 (m, 2H, ArH), 8.25-8.15 (m, 1H, ArH).

### Example 14: (2S)-Isopropyl 2-(((4-(aminomethyl)-5-hydroxy-6-methylpyridin-3-yl)methoxy)(4-(trifluoromethoxy)phenoxy)phosphorylamino)propanoate (14xi)

¹H NMR (299.992 MHz, methanol-d₄) δ 0.95-1.04 (m, 6H, 2CH₃), 1.11-1.19 (m, 3H, CH₃), 2.54 (s, 3H, CH₃), 3.66-3.82 (m, 1H, CH), 4.11-4.25 (m, 2H, CH₂), 4.66-4.84 (m, 1H, CH), 5.18-5.33 (m, 2H, CH₂), 6.96-7.17 (m, 4H, ArH), 8.11-8.22 (m, 1H, ArH).

### Example 15: (2S)-5,6,7,8-Tetrahydronaphthalen-2-yl 2-(((4-(aminomethyl)-5-hydroxy-6-methylpyridin-3-yl)methoxy)(phenoxy)phosphorylamino)propanoate (14xii)

¹H NMR (299.992 MHz, methanol-d₄) δ 1.21 (s, 3H, CH₃), 1.36-1.46 (m, 3H, CH₃), 1.63-1.78 (m, 4H, 2CH₂), 2.60-2.72 (m, 4H, 2CH₂), 4.04-4.26 (m, 2H, CH₂), 5.22-5.38 (m, 2H, CH₂), 6.56-6.69 (m, 2H, ArH), 6.90-6.99 (m, 1H, ArH), 7.09-7.21 (m, 2H, ArH), 7.24-7.34 (m, 2H, ArH), 8.13-8.21 (m, 1H, ArH).

### Example 16: Pyridoxamine Prodrug 14 with Deuterated Methylene Group and Methyl Groups of the Isopropyl Group (14xiii)

¹H NMR (299.992 MHz, methanol-d₄) δ 2.57 (s, 3H, ArCH₃), 4.14-4.38 (m, 2H, CH₂), 4.67-4.84 (m, 1H, NH), 5.28-5.39 (m, 2H, CH₂), 7.13-7.27 (m, 3H, ArH), 7.29-7.42 (m, 2H, ArH), 7.95-8.14 (m, 1H, ArH).

### Example 17: (2S)-Isopropyl 2-(((4-(aminomethyl)-5-hydroxy-6-methylpyridin-3-yl)methoxy)(phenoxy)phosphorylamino)-3-(4-(benzyloxy)phenyl)propanoate (14xiv)

¹H NMR (299.992 MHz, methanol-d₄) δ1.04-1.24 (m, 6H, 2CH₃), 2.55-2.72 (m, 3H, ArCH₃), 2.74-2.88 (m, 1H, CH), 2.90-3.12 (m, 1H, CH), 3.93-4.11 (m, 1H, NH), 4.14-4.31 (m, 2H, CH₂), 4.77-5.38 (m, 9H, CH, NH₂, CH₂), 6.79-6.94 (m, 2H, ArH), 6.95-7.20 (m, 5H, ArH), 7.20-7.46 (m, 7H, ArH), 7.98-8.23 (m, 1H, ArH).

### Example 18: (2S)-1-Methylpiperidin-4-yl 2-(((4-(aminomethyl)-5-hydroxy-6-methylpyridin-3-yl)methoxy)(phenoxy)phosphorylamino)propanoate (14xv)

¹H NMR (299.992 MHz, methanol-d₄) δ1.51-1.36 (m, 3H, CH₃), 2.30-1.80 (m, 4H, 2CH₂), 2.75-2.69 (m, 3H, ArCH₃), 2.93-2.84 (m, 3H, ArCH₃), 3.66-3.10 (m, 4H, 2CH₂), 4.17-3.97 (m, 1H, CH), 4.41-4.27 (m, 2H, CH₂), 5.19-4.91 (m, 1H, CH), 5.51-5.33 (m, 2H, CH₂), 7.45-7.17 (m, 5H, ArH), 8.36-8.24 (m, 1H, ArH).

### Example 19: (2S)-Isopropyl 2-(((4-(aminomethyl)-5-hydroxy-6-methylpyridin-3-yl)methoxy)(p-tolyloxy)phosphorylamino)propanoate (14xvi)

¹H NMR (299.992 MHz, methanol-d₄) δ1.25-1.17 (m, 6H, 2CH₃), 1.36-1.30 (m, 3H, CH₃), 2.32 (s, 3H, ArCH₃), 2.60 (s, 3H, ArCH₃), 3.96-3.83 (m, 1H, CH), 4.32-4.23 (m, 2H, CH₂), 5.03-4.80 (m, 1H, CH), 5.38-5.24 (m, 2H, CH₂), 7.18-7.03 (m, 5H, ArH), 8.16-8.07 (m, 1H, ArH).

### Example 20: (2S)-4-Chlorophenyl 2-(((4-(aminomethyl)-5-hydroxy-6-methylpyridin-3-yl)methoxy)(phenoxy)phosphorylamino)propanoate (14xvii)

¹H NMR (299.992 MHz, methanol-d₄) δ1.35-1.46 (m, 3H, CH₃), 2.44 (br, 3H, ArCH₃), 4.02-4.17 (m, 3H, CH, CH₂), 5.14-5.26 (m, 2H, CH₂), 6.83-6.98 (m, 2H, ArH), 7.05-7.18 (m, 3H, ArH), 7.19-7.33 (m, 4H, ArH), 7.88-7.95 (m, 1H, ArH).

### Example 21: (2S)-Isopropyl 2-(((4-(aminomethyl)-5-hydroxy-6-methylpyridin-3-yl)methoxy)(4-bromo-2-fluorophenoxy)phosphorylamino)propanoate (14xviii)

¹H NMR (299.992 MHz, CDCl₃) 51.06-1.17 (s, 6H, 2CH₃), 1.22-1.33 (m, 3H, CH₃), 2.49-2.65 (m, 3H, CH₃), 3.69-3.95 (m, 1H, CH), 4.09-4.36 (m, 2H, CH₂), 4.53-4.77 (m, 1H, CH), 5.14-5.55 (m, 2H, CH₂), 7.16-7.30 (m, 2H, ArH), 7.33-7.44 (m, 1H, ArH), 8.08-8.19 (m, 1H, ArH).

### Synthesis of Pyridoxine Prodrugs

### Example 22: (2S)-Isopropyl 2-(((5-hydroxy-4-(hydroxymethyl)-6-methylpyridin-3-yl)methoxy)(phenoxy)phosphorylamino)propanoate (15)

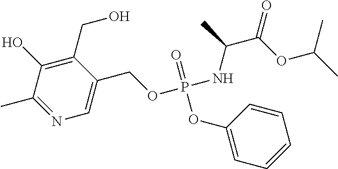 [SMILES: Cc1ncc(COP(=O)(N[C@@H](C)C(=O)OC(C)C)Oc2ccccc2)c(CO)c1O]

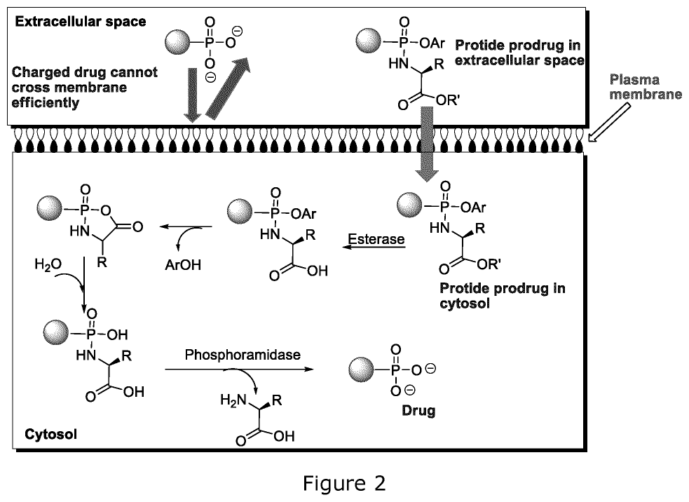

Compound 5 (P-rac, S) (1.15 equiv.), MgCl₂ (1 equiv.), and compound 6 (1 equiv.) were suspended in dry acetonitrile and under argon, the mixture was stirred and heated to 50° C. for 10 minutes. DIPEA (2.5 equiv.) was then added and stirring was continued for a further 30 minutes. Solvents were removed under vacuum and purification using biotage (hex/EtOAc) gave acetonide-protected intermediate 7. After drying, compound 7 was dissolved in dry THF and cooled to 0° C. Concentrated HCl (12 equiv.) was added slowly at 0° C. When the addition was complete, the reaction mixture was allowed to warm to rt with stirring. When Mass and HPLC indicated that the reaction had progressed to 50%, the reaction was quenched by adding excess TEA slowly at-78° C. The solid salts were filtered off and rinsed using EtOAc. The liquid filtrates were combined, concentrated, and purification using biotage (EtOAc or DCM/MeOH) gave pure unreacted acetonide-protected and the desired alcohol product. ¹H NMR (299.992 MHz, chloroform-d) δ1.08-1.47 (m, 9H, 3CH₃), 2.48 (s, 3H, ArCH₃), 3.79-4.23 (m, 2H, CH, NH), 4.82-5.17 (m, 5H, 2CH₂, CH), 7.07-7.41 (m, 5H, ArH), 7.83-7.97 (m, 1H, ArH). The pyridoxine prodrugs depicted in FIG. 2 were all synthesized following the same procedure.

### Characterization Data for Selected Alcohols

### Example 23: (S)-Isopropyl 2-((S)-((5-hydroxy-4-(hydroxymethyl)-6-methylpyridin-3-yl)methoxy)(phenoxy)phosphorylamino)propanoate (15i)

¹H NMR (299.992 MHz, chloroform-d) δ1.14-1.33 (m, 6H, 2CH₃), 1.34-1.46 (m, 3H, CH₃), 2.56 (s, 3H, ArCH₃), 3.87-4.09 (m, 2H, NH, CH), 4.92-5.20 (m, 5H, 2CH₂, CH), 7.19-7.31 (m, 3H, ArH), 7.33-7.45 (m, 2H, ArH), 7.99 (s, 1H, ArH).

### Example 24: (S)-Isopropyl 2-((R)-((5-hydroxy-4-(hydroxymethyl)-6-methylpyridin-3-yl)methoxy)(phenoxy)phosphorylamino)propanoate (15ii)

¹H NMR (299.992 MHz, chloroform-d) δ1.10-1.24 (m, 6H, 2CH₃), 1.26-1.37 (m, 3H, CH₃), 2.45 (s, 3H, ArCH₃), 3.76-3.98 (m, 1H, CH), 4.35-4.56 (m, 1H, NH), 4.82-5.13 (m, 5H, 2CH₂, CH), 7.08-7.22 (m, 3H, ArH), 7.23-7.36 (m, 2H, ArH), 7.86 (s, 1H, ArH).

### Example 25: (2S)-Isopropyl 2-((3,4-dichlorophenoxy)((5-hydroxy-4-(hydroxymethyl)-6-methylpyridin-3-yl)methoxy)phosphorylamino)propanoate (15iii)

¹H NMR (299.992 MHz, chloroform-d) δ1.05-1.29 (m, 9H, 3CH₃), 2.38 (s, 1H, CH₃), 3.70-3.88 (s, 1H, CH), 4.06-4.32 (m, 1H, CH), 4.79-5.05 (m, 5H, 2CH₂, NH), 6.25-6.86 (br, 2H, 20H), 6.91-6.99 (m, 1H, ArH), 7.15-7.23 (m, 1H, ArH), 7.24-7.30 (m, 1H, ArH), 7.78-7.85 (m, 1H, ArH).

### Example 26: (2S)-Naphthalen-2-yl 2-(((5-hydroxy-4-(hydroxymethyl)-6-methylpyridin-3-yl)methoxy)(phenoxy)phosphorylamino)propanoate (15iv)

¹H NMR (299.992 MHz, chloroform-d) δ1.37-1.57 (m, 3H, CH₃), 2.41 (br, 3H, ArCH₃), 4.06-4.41 (m, 2H, NH, CH), 4.64-5.14 (m, 4H, 2CH₂), 6.75-7.96 (m, 13H, ArH).

### Example 27: Isopropyl 2-(((5-hydroxy-4-(hydroxymethyl)-6-methylpyridin-3-yl)methoxy)(phenoxy)phosphorylamino)acetate (15v)

¹H NMR (299.992 MHz, chloroform-d) δ1.18-1.37 (m, 6H, 2CH₃), 2.56 (s, 3H, ArCH₃), 3.57-3.87 (m, 2H, CH₂), 3.94-4.18 (m, 1H, NH), 4.79-5.29 (m, 5H, ArH), 7.99 (s, 1H, ArH).

### Example 28: (2S)-Methyl 2-(((5-hydroxy-4-(hydroxymethyl)-6-methylpyridin-3-yl)methoxy)(phenoxy)phosphorylamino)propanoate (15vi)

¹H NMR (299.992 MHz, chloroform-d) δ 1.28-1.42 (m, 3H, CH₃), 2.62 (s, 3H, CH₃), 3.59-3.81 (m, 4H, CH₂, CH, OH), 3.90-4.13 (br, 1H, OH), 4.87-5.37 (m, 3H, CH, CH₂), 7.06-7.41 (m, 5ArH), 8.03 (s, 1H, ArH).

### Example 29: (2S)-Cyclopentylmethyl 2-(((5-hydroxy-4-(hydroxymethyl)-6-methylpyridin-3-yl)methoxy)(phenoxy)phosphorylamino)propanoate (15vii)

¹H NMR (299.992 MHz, chloroform-d) δ1.04-1.37 (m, 7H, CH₂), 1.43-1.78 (m, 8H, CH₂, CH₃), 1.96-2.21 (m, 1H, CH), 2.44 (s, 3H, ArCH₃), 3.78-4.04 (m, 4H, CH, NH, CH₂), 4.78-5.08 (m, 4H, CH, CH₂), 7.04-7.20 (m, 3H, ArH), 7.20-7.35 (m, 2H, ArH), 7.79-7.92 (m, 1H, ArH).

### Example 30: (2S)-Isopropyl 2-((2,3-dihydro-1H-inden-5-yloxy)((5-hydroxy-4-(hydroxymethyl)-6-methylpyridin-3-yl)methoxy)phosphorylamino)propanoate (15viii)

¹H NMR (299.992 MHz, chloroform-d) δ1.09-1.21 (m, 6H, 2CH₃), 1.23-1.33 (m, 3H, CH₃), 1.94-2.11 (m, 2H, CH₂), 2.43 (s, 3H, CH₃), 2.76-2.86 (m, 4H, 2CH₂), 3.74-4.00 (m, 2H, CH, NH), 4.82-5.01 (m, 5H, 2CH₂, CH), 6.01-6.88 (m, 1H, ArH), 6.90-6.98 (m, 1H, ArH), 7.01-7.09 (m, 1H, ArH), 7.82-7.88 (m, 1H, ArH).

### Example 31: (2S)-Methyl 2-((4-chlorophenoxy)((5-hydroxy-4-(hydroxymethyl)-6-methylpyridin-3-yl)methoxy)phosphorylamino)propanoate (15ix)

¹H NMR (299.992 MHz, chloroform-d) δ1.13-1.42 (m, 9H, 3CH₃), 2.62 (s, 3H, ArCH₃), 3.80-4.12 (br, 1H, CH), 4.78-5.39 (m, 5H, CH₂, CH), 7.09-7.36 (m, 5H, ArH), 7.98-8.16 (m, 1ArH).

### Example 32: (2S)-Isopropyl 2-(((5-hydroxy-4-(hydroxymethyl)-6-methylpyridin-3-yl)methoxy)(naphthalen-1-yloxy)phosphorylamino)propanoate (15x)

¹H NMR (299.992 MHz, chloroform-d) δ1.20-1.23 (m, 6H, CH₃), 1.37-1.40 (m, 3H, CH₃), 2.55 (s, 3H, ArCH₃), 3.99-4.09 (m, 1H, CH), 4.89-5.02 (m, 4H, CH₂, NH, CH), 5.26-5.34 (m, 2H, CH₂), 7.41-7.44 (m, 2H, ArH), 7.51-7.58 (m, 2H, ArH), 7.69-7.72 (m, 1H, ArH), 7.86-7.96 (m, 2H, ArH), 8.07-8.12 (m, 1H, ArH).

### Example 33: (2S)-Benzyl 2-(((5-hydroxy-4-(hydroxymethyl)-6-methylpyridin-3-yl)methoxy)(phenoxy)phosphorylamino)propanoate (15xi)

¹H NMR (299.992 MHz, chloroform-d) δ1.17-1.38 (m, 3H, 3CH₃), 2.39 (br, 3H, ArCH₃), 3.86-4.14 (m, 1H, OH), 4.54-4.76 (m, 1H, CH), 4.80-5.16 (m, 6H, 3CH₂), 6.96-7.39 (m, 9H, ArH), 7.52-8.69 (m, 3H, ArH, NH, ArH).

### Example 34: (2S)-Isopropyl 2-cyclopentyl-2-(((5-hydroxy-4-(hydroxymethyl)-6-methylpyridin-3-yl)methoxy)(phenoxy)phosphorylamino)acetate (15xii)

¹H NMR (299.992 MHz, chloroform-d) δ1.17-1.36 (m, 6H, 2CH₃), 1.33-1.50 (m, 2H, CH), 1.51-1.82 (m, 6H, CH₂), 2.09-2.31 (m, 1H, CH), 2.56 (s, 3H, ArCH₃), 3.64-3.86 (m, 1H, CH), 3.98-4.30 (m, 1H, NH), 4.88-5.21 (m, 5H, CH, CH₂), 7.17-7.32 (m, 3H, ArH), 7.32-7.46 (m, 2H, ArH), 7.89-8.02 (m, 1H, ArH).

### Example 35: (2S)-Isopropyl 2-(((5-hydroxy-4-(hydroxymethyl)-6-methylpyridin-3-yl)methoxy)(phenoxy)phosphorylamino)-4-methylpentanoate (15xiii)

¹H NMR (299.992 MHz, chloroform-d) δ0.72-0.90 (m, 6H, CH₃), 1.00-1.21 (m, 6H, CH₃), 1.29-1.77 (m, 4H, CH₂), 2.43 (s, 3H, ArCH₃), 3.61-4.00 (m, 2H, NH, CH), 4.74-5.10 (m, 5H, CH, CH₂), 7.04-7.14 (m, 3H, ArH), 7.20-7.34 (m, 2H, ArH), 7.77-7.93 (m, 1H, ArH), 8.61-9.34 (brs s, 1H).

### Example 36: Isopropyl 4,4,4-trifluoro-2-(((5-hydroxy-4-(hydroxymethyl)-6-methylpyridin-3-yl)methoxy)(phenoxy)phosphorylamino)butanoate (15xiv)

¹H NMR (299.992 MHz, chloroform-d) δ1.25-1.39 (m, 6H, 2CH₃), 2.57 (s, 3H, CH₃), 2.59-2.81 (m, 2H, CH₂), 4.21-4.36 (m, 1H, CH), 4.48-4.72 (m, 1H, CH), 4.96-5.20 (m, 5H, CH, CH₂), 7.20-7.33 (m, 3H, ArH), 7.39-7.47 (m, 2H, ArH), 7.95-8.03 (m, 1H, ArH).

### Example 37: Tert-butyl 3-((2S)-2-(((5-hydroxy-4-(hydroxymethyl)-6-methylpyridin-3-yl)methoxy)(phenoxy) phosphorylamino)-3-isopropoxy-3-oxopropyl)-1H-indole-1-carboxylate (15xv)

¹H NMR (299.992 MHz, chloroform-d) δ0.97-1.28 (m, 6H, 2CH₃), 1.70 (s, 9H, 3CH₃), 2.51 (s, 3H, ArCH₃), 2.94-3.32 (m, 2H, CH), 3.70-4.38 (m, 2H, NH, CH), 4.72-5.08 (m, 5H, CH, CH₂), 6.93-7.61 (m, 10H, ArH), 7.78-7.98 (m, 1H, ArH), 8.04-8.21 (m, 1H, ArH).

### Example 38: (2S)-Isopropyl 3-(4-(benzyloxy)phenyl)-2-(((5-hydroxy-4-(hydroxymethyl)-6-methylpyridin-3-yl)methoxy)(phenoxy)phosphorylamino)propanoate (15xvi)

¹H NMR (299.992 MHz, chloroform-d) δ0.96-1.21 (m, 6H, 2CH₃), 2.44 (s, 3H, CH₃), 2.74-2.97 (m, 2H, CH₂), 3.63-3.87 (m, 1H, CH), 3.89-4.13 (m, 1H, NH), 4.55-4.94 (m, 5H, CH, CH₂), 4.99 (s, 2H, CH₂), 6.77-6.88 (m, 2H, ArH), 6.93-7.15 (m, 5H, ArH), 7.18-7.43 (m, 7H, ArH), 7.98-7.89 (m, 1H, ArH).

### Example 39: (2S)-Isopropyl 2-(((5-benzoyloxy-4-(hydroxymethyl)-6-methylpyridin-3-yl)methoxy)(phenoxy)phosphorylamino)propanoate (15xvii)

¹H NMR (299.992 MHz, chloroform-d) δ1.00-1.07 (m, 6H, CH₃), 1.14-1.23 (m, 3H, CH₃), 2.39 (s, 3H, ArCH₃), 3.51-3.64 (m, 1H, CH), 3.76-3.86 (m, 1H, NH), 4.79-4.85 (m, 1H, CH), 5.11-5.16 (m, 2H, CH₂), 5.26-5.29 (m, 2H, CH₂), 6.96-7.17 (m, 6H, ArH), 7.22-7.32 (m, 3H, ArH), 7.39-7.45 (m, 1H, ArH), 7.87-8.00 (m, 3H, ArH).

### Example 40: (2S)-Isopropyl 2-(((5-(dimethylcarbamoyloxy)-4-((dimethylcarbamoyloxy)methyl)-6-methylpyridin-3-yl)methoxy)(phenoxy)phosphorylamino)propanoate (15xviii)

¹H NMR (299.992 MHz, chloroform-d) δ1.18-1.23 (m, 6H, CH₃), 1.29-1.37 (m, 3H, CH₃), 2.45 (s, 3H, ArCH₃), 2.81-2.89 (m, 6H, NCH₃), 3.01 (s, 3H, NCH₃), 3.17 (s, 3H, NCH₃), 3.68-3.77 (m, 1H, CH), 3.91-3.99 (m, 1H, NH), 4.95-5.01 (m, 1H, CH), 5.11-5.14 (m, 2H, CH2), 5.28-5.34 (m, 2H, CH₂), 7.10-7.20 (m, 3H, ArH), 7.26-7.33 (m, 3H, ArH), 8.37-8.41 (m, 1H, ArH).

### Example 41: Pyridoxine Prodrug 15 with a Deuterated Benzene Ring (15xix)

¹H NMR (299.992 MHz, chloroform-d) δ1.08-1.23 (m, 6H, 2CH₃), 1.22-1.35 (m, 3H, CH₃), 2.43 (s, 3H, ArCH₃), 3.76-4.02 (m, 2H, 2CH), 4.72-5.12 (m, 5H, NH, 2CH₂), 7.78-7.91 (s, 1H, ArH).

### Example 42: Pyridoxine Prodrug 15 with a Deuterated Isopropyl Group (15xx)

¹H NMR (299.992 MHz, chloroform-d) δ 1.31-1.55 (m, 3H, CH₃), 2.58 (s, 3H, ArCH₃), 3.88-4.11 (m, 1H, CH), 4.18-4.46 (m, 1H, NH), 4.94-5.27 (m, 4H, 2CH₂), 7.11-7.34 (m, 3H, ArH), 7.34-7.47 (m, 2H, ArH), 7.94-8.02 (m, 1H, ArH).

### Example 43: (2R)-Isopropyl 2-(((5-hydroxy-4-(hydroxymethyl)-6-methylpyridin-3-yl)methoxy)(phenoxy)phosphorylamino)propanoate (15xxi)

¹H NMR (299.992 MHz, chloroform-d) δ1.18-1.51 (m, 9H, 3CH₃), 2.58 (s, 3H, ArCH₃), 3.89-4.24 (m, 2H, CH, NH), 4.95-5.24 (m, 5H, 2CH₂, CH), 7.17-7.49 (m, 5H, ArH), 7.96-8.06 (m, 1H, ArH).

### Example 44: (2S)-Cyclopentyl 2-(((5-hydroxy-4-(hydroxymethyl)-6-methylpyridin-3-yl)methoxy)(phenoxy)phosphorylamino)propanoate (15xxii)

¹H NMR (299.992 MHz, chloroform-d) δ1.33-1.24 (m, 3H, CH₃), 1.90-1.45 (m, 8H, 4CH₂), 2.49-2.33 (m, 3H, ArCH₃), 3.95-3.72 (m, 2H, NH, CH), 4.54 (br s, 1H, OH), 5.21-4.83 (m, 5H, CH, 2CH₂), 7.34-7.05 (m, 5H, ArH), 7.92 (s, 1H, ArH).

### Example 45: Pyridoxine Prodrug 15 with Deuterated Methyl Groups of the Isopropyl Group (15xxiii)

¹H NMR (299.992 MHz, chloroform-d) δ 1.15-1.37 (m, 3H, CH₃), 2.45 (s, 3H, ArCH₃), 3.68-3.95 (m, 2H, 2CH), 4.77-5.17 (m, 5H, 2CH₂, NH), 7.02-7.19 (m, 3H, ArH), 7.22-7.37 (m, 2H, ArH), 7.94-8.02 (s, 1H, ArH).

### Example 46: (2S)-p-Tolyl 2-(((5-hydroxy-4-(hydroxymethyl)-6-methylpyridin-3-yl)methoxy)(phenoxy)phosphorylamino)propanoate (15xxiv)

¹H NMR (299.992 MHz, chloroform-d) δ1.48-1.39 (m, 3H, CH₃), 2.48-2.26 (m, 6H, 2 ArCH₃), 4.24-4.04 (m, 2H, NH, CH), 4.50 (br s, 1H, OH), 5.07-4.80 (m, 4H, 2CH₂), 6.86-6.68 (m, 2H, ArH), 7.32-7.05 (m, 7H, ArH), 7.85 (s, 1H, ArH).

### Example 47: (2S)-Isopropyl 2-(((5-hydroxy-4-(hydroxymethyl)-6-methylpyridin-3-yl)methoxy)(4-(trifluoromethoxy)phenoxy)phosphorylamino)propanoate (15xxv)

¹H NMR (299.992 MHz, chloroform-d) δ1.16-1.51 (m, 9H, 3CH₃), 2.54 (s, 3H, CH₃), 3.90-4.07 (m, 1H, CH), 4.45-4.67 (m, 1H, CH), 4.96-5.22 (m, 4H, 2CH₂), 7.15-7.35 (m, 4H, ArH), 7.43-7.88 (br, 1H, NH), 7.93-8.04 (m, 1H, ArH).

### Example 48: (2S)-Isopropyl 2-(((5-hydroxy-4-(hydroxymethyl)-6-methylpyridin-3-yl)methoxy)(5,6,7,8-tetrahydronaphthalen-1-yloxy)phosphorylamino)propanoate (15 xxvi)

¹H NMR (299.992 MHz, chloroform-d) δ1.41-1.49 (s, 3H, CH₃), 1.69-1.82 (m, 4H, 2CH₂), 2.41-2.47 (m, 3H, CH₃), 2.62-2.76 (m, 4H, 2CH₂), 3.95-4.23 (m, 2H, NH, CH), 4.80-5.09 (m, 4H, 2CH₂), 6.50-6.71 (m, 2H, ArH), 6.93-7.03 (m, 1H, ArH), 7.08-7.34 (m, 5H, ArH), 7.85-7.91 (m, 1H, ArH).

### Example 49: Pyridoxine Prodrug 15 with Deuterated Methylene Group (15xxvii)

¹H NMR (299.992 MHz, chloroform-d) δ 1.08-1.20 (m, 6H, 2CH₃), 1.21-1.33 (m, 3H, CH₃), 2.43 (s, 3H, ArCH₃), 3.78-4.06 (m, 2H, 2CH), 4.79-5.07 (m, 3H, CH₂, NH), 7.04-7.18 (m, 3H, ArH), 7.20-7.31 (m, 2H, ArH), 7.81-7.89 (s, 1H, ArH).

### Example 50: (2S)-5,6,7,8-Tetrahydronaphthalen-2-yl 2-(((5-hydroxy-4-(hydroxymethyl)-6-methylpyridin-3-yl)methoxy)(phenoxy)phosphorylamino)propanoate (15xxviii)

¹H NMR (299.992 MHz, CDCl₃) 51.41-1.49 (s, 3H, CH₃), 1.69-1.82 (m, 4H, 2CH₂), 2.41-2.47 (m, 3H, CH₃), 2.62-2.76 (m, 4H, 2CH₂), 3.95-4.23 (m, 2H, NH, CH), 4.80-5.09 (m, 4H, 2CH₂), 6.50-6.71 (m, 2H, ArH), 6.93-7.03 (m, 1H, ArH), 7.08-7.34 (m, 5H, ArH), 7.85-7.91 (m, 1H, ArH).

### Example 51: (2S)-1-Methylpiperidin-4-yl 2-(((5-hydroxy-4-(hydroxymethyl)-6-methylpyridin-3-yl)methoxy)(phenoxy)phosphorylamino)propanoate (15xxix)

¹H NMR (299.992 MHz, methanol-d₄) δ1.41-1.25 (m, 3H, CH₃), 2.05-1.62 (m, 4H, 2CH₂), 2.57-3.32 (m, 8H, CH₂, NCH₃, ArCH₃), 2.84-2.66 (m, 2H, CH₂), 3.99-3.85 (m, 1H, CH), 4.96-4.71 (m, 3H, CH, CH₂), 5.23-5.10 (m, 2H, CH₂), 7.41-7.12 (m, 5H, ArH), 7.93-7.84 (m, 1H, ArH).

### Example 52: (2S)-Methyl 1-(((5-hydroxy-4-(hydroxymethyl)-6-methylpyridin-3-yl)methoxy)(phenoxy)phosphoryl)pyrrolidine-2-carboxylate (15xxx)

¹H NMR (299.992 MHz, CDCl₃) 51.71-2.24 (m, 4H, Proline), 2.46 (s, 3H, ArCH₃), 3.12-3.45 (m, 3H. Proline), 3.50-3.74 (m, 3H, COOCH₃), 4.02-4.32 (m, 1H, CH), 4.69-5.21 (m, 5H, two CH₂, NH), 7.01-7.20 (m, 3H, ArH), 7.22-7.36 (m, 2H, ArH), 7.89-8.01 (m, 1H, ArH).

### Example 53: Pyridoxine Prodrug 15 with Deuterated Methylene and Methyl Groups (15xxvi)

¹H NMR (299.992 MHz, chloroform-d) δ 1.23-1.37 (m, 6H, 2CH₃) 1.38-1.52 (m, 3H, CH₃), 3.92-4.11 (m, 1H, CH), 4.183-4.29 (m, 1H, CH), 4.97-5.21 (m, 3H, CH₂, NH), 7.19-7.34 (m, 3H, ArH), 7.36-7.47 (m, 2H, ArH), 7.94-8.06 (m, 1H, ArH).

### Example 54: (2S)-Isopropyl 2-(((5-hydroxy-4-(hydroxymethyl)-6-methylpyridin-3-yl)methoxy)(p-tolyloxy)phosphorylamino)propanoate (15xxxii)

¹H NMR (299.992 MHz, chloroform-d) δ1.21-1.12 (m, 6H, 2CH₃), 1.31-1.26 (m, 3H, CH₃), 2.30-2.26 (m, 3H, ArCH₃), 2.45 (s, 3H, ArCH₃), 3.94-3.72 (m, 2H, NH, CH), 5.04-4.83 (m, 5H, CH, 2CH₂), 7.10-7.93 (m, 4H, ArH), 7.91-7.84 (m, 1H, ArH).

### Example 55: (2S)-Isopropyl 2-((4-bromo-2-fluorophenoxy)((5-hydroxy-4-(hydroxymethyl)-6-methylpyridin-3-yl)methoxy)phosphorylamino)propanoate (15xxxiii)

¹H NMR (299.992 MHz, Chloroform-d) δ1.35-1.09 (m, 9H, 3CH₃), 2.45 (s, 3H, ArCH₃), 4.08-3.79 (m, 2H, NH, CH), 5.14-4.84 (m, 5H, CH, 2CH₂), 7.32-7.15 (m, 3H, ArH), 7.93-7.86 (m, 1H, ArH)

### Example 56: (2S)-Isopropyl 2-(((4-formyl-5-hydroxy-6-methylpyridin-3-yl)methoxy)(phenoxy)phosphorylamino)propanoate (16)

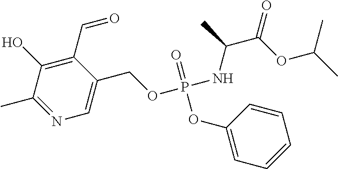 [SMILES: Cc1ncc(COP(=O)(N[C@@H](C)C(=O)OC(C)C)Oc2ccccc2)c(C=O)c1O]

MnO₂ (12 equiv.) was suspended in dry DCM and the mixture was cooled to 0° C. After flushing the system using argon, the alcohol (1 equiv.) dissolved in DCM was then injected into the flask under argon. The ice bath was removed and stirring was continued for a further 1 to 2 hrs when TLC confirmed the disappearance of the starting material. The mixture was filtered through a short silica gel column (EtOAc) to remove excess MnO₂ and to give the product as a pale yellow thick oil. ¹H NMR (299.992 MHz, chloroform-d) δ1.15-1.53 (m, 9H, 3CH₃), 2.59 (br, 3H, ArCH₃), 3.67-4.13 (m, 2H, CH, NH), 4.94-5.13 (m, 1H, CH), 5.32-5.54 (m, 2H, CH₂), 7.10-7.43 (m, 5H, ArH), 8.09-8.22 (m, 1H, ArH), 10.29-10.46 (m, 1H, CH), 11.31-11.73 (br, 1H, ArOH). The pyridoxal prodrugs depicted in FIGS. 2 and 3 were all synthesized following the same procedure.

### Characterization Data for Selected Aldehydes

### Example 57: (S)-Isopropyl 2-((S)-((4-formyl-5-hydroxy-6-methylpyridin-3-yl)methoxy)(phenoxy)phosphorylamino)propanoate (16i)

¹H NMR (299.992 MHz, chloroform-d) δ1.16-1.36 (m, 6H, 2CH₃), 1.38-1.54 (m, 3H, CH₃), 2.63 (s, 3H, ArCH₃), 3.88-4.13 (m, 2H, CH, NH), 5.01-5.15 (m, 1H, CH), 5.36-5.55 (m, 2H, CH₂), 7.16-7.46 (m, 5H, ArH), 8.18 (s, 1H, ArH), 10.41 (s, 1H, CH), 11.59 (br, 1H, ArOH).

### Example 58: (S)-Isopropyl 2-((R)-((4-formyl-5-hydroxy-6-methylpyridin-3-yl)methoxy)(phenoxy)phosphorylamino)propanoate (16ii)

¹H NMR (299.992 MHz, chloroform-d) δ1.19-1.29 (m, 6H, 2CH₃), 1.36-1.44 (m, 3H, CH₃), 2.59 (s, 3H, ArCH₃), 3.80-4.04 (m, 2H, CH, NH), 4.94-5.11 (m, 1H, CH), 5.31-5.49 (m, 2H, CH₂), 7.13-7.26 (m, 3H, ArH), 7.27-7.41 (m, 2H, ArH), 8.14 (s, 1H, ArH), 10.36 (s, 1H, CHO), 11.55 (br, 1H, ArOH).

### Example 59: (2R)-Isopropyl 2-(((4-formyl-5-hydroxy-6-methylpyridin-3-yl)methoxy)(phenoxy)phosphorylamino)propanoate (16iii)

¹H NMR (299.992 MHz, chloroform-d) δ1.17-1.51 (m, 9H, 3CH₃), 2.56-2.64 (br, 3H, ArCH₃), 3.69-4.11 (m, 2H, CH, NH), 4.95-5.14 (m, 1H, CH), 5.29-5.53 (m, 2H, CH₂), 7.12-7.42 (m, 5H, ArH), 8.08-8.21 (m, 1H, ArH), 10.31-10.45 (m, 1H, CH), 11.55 (br, 1H, ArOH).

### Example 60: (2S)-Isopropyl 2-(((4-formyl-5-hydroxy-6-methylpyridin-3-yl)methoxy)(naphthalen-1-yloxy)phosphorylamino)propanoate (16iv)

¹H NMR (299.992 MHz, chloroform-d) δ1.21-1.26 (m, 6H, CH₃), 1.44-1.46 (m, 3H, CH₃), 2.73-2.74 (m, 3H, ArCH₃), 4.20-4.31 (m, 1H, CH), 4.84-5.08 (m, 2H, NH, CH), 5.28-5.52 (m, 2H, CH₂), 7.28-7.51 (m, 4H, ArH), 7.60-7.78 (m, 2H, ArH), 7.89-8.10 (m, 2H, ArH), 10.18 (s, 1H, CHO).

### Example 61: (2S)-2-Ethylbutyl 2-(((4-formyl-5-hydroxy-6-methylpyridin-3-yl)methoxy)(phenoxy)phosphorylamino)propanoate (16v)

¹H NMR (299.992 MHz, chloroform-d) δ0.76-0.98 (m, 6H, 2CH₃), 1.21-1.66 (m, 9H, CH, CH₂), 2.82 (s, 3H, ArCH₃), 3.88-4.21 (m, 3H, NH, CH₂), 4.27-4.53 (m, 1H, CH), 5.34-5.75 (m, 2H, CH₂), 7.07-7.41 (m, 5H, ArH), 8.21-8.46 (m, 1H, ArH), 10.36-10.57 (m, 1H, CHO);

### Example 62: (2S)-Isopropyl 2-(((4-formyl-5-hydroxy-6-methylpyridin-3-yl)methoxy)(phenoxy)phosphorylamino)-3-phenylpropanoate (16vi)

¹H NMR (299.992 MHz, chloroform-d) δ1.08-1.21 (m, 6H, 2CH₃), 2.52 (s, 3H, ArCH₃), 2.86-3.05 (m, 2H, CH₂), 3.44-3.59 (m, 1H, CH), 4.02-4.27 (m, 1H, NH), 4.78-5.13 (m, 2H, CH₂), 5.14-5.25 (m, 1H, CH), 7.00-7.17 (m, 4H, ArH), 7.18-7.31 (m, 5H, ArH), 7.92-8.03 (m, 1H, ArH), 10.13-10.23 (m, 1H, CHO).

### Example 63: (2S)-Isopropyl 2-(((4-formyl-5-hydroxy-6-methylpyridin-3-yl)methoxy)(phenoxy)phosphorylamino)-4-methylpentanoate (16vii)

¹H NMR (299.992 MHz, chloroform-d) δ0.87-1.05 (m, 6H, 2CH₃), 1.19-1.36 (m, 6H, 2CH₃), 1.42-1.90 (m, 3H, CH, CH₂), 2.63 (s, 3H, ArCH₃), 3.58-4.05 (m, 2H, NH, CH), 4.95-5.13 (m, 1H, CH), 5.32-5.54 (m, 2H, CH₂), 7.14-7.29 (m, 3H, ArH), 7.30-7.42 (m, 2H, ArH), 8.11-8.24 (m, 1H, ArH, 10.31-10.42 (m, 1H, CHO).

### Example 64: (2S)-Isopropyl 2-((4-chlorophenoxy)((4-formyl-5-hydroxy-6-methylpyridin-3-yl)methoxy)phosphorylamino)propanoate (16viii)

¹H NMR (299.992 MHz, chloroform-d) δ1.20-1.29 (m, 6H, 2CH₃), 1.34-1.42 (m, 3H, CH₃), 2.59 (s, 3H, CH₃), 3.64-3.83 (m, 1H, CH), 3.86 (m, 1H, CH), 4.96 (m, 1H, NH), 7.07-7.16 (m, 2H, ArH), 7.23-7.32 (m, 2H, ArH), 8.01-8.17 (m, 1H, ArH), 10.33-10.41 (m, 1H, CHO) 11.52 (s, 1H, OH).

### Example 65: (2S)-Benzyl 2-(((4-formyl-5-hydroxy-6-methylpyridin-3-yl)methoxy)(phenoxy)phosphorylamino)propanoate (16ix)

¹H NMR (299.992 MHz, chloroform-d) δ1.23-1.40 (m, 3H, CH₃), 2.40-2.57 (m, 3H, ArCH₃), 3.75-4.12 (m, 2H, CH, NH), 4.93-5.36 (m, 4H, 2CH₂), 6.95-7.38 (m, 10H, ArH), 7.95-8.07 (m, 1H, ArH), 10.15-10.31 (m, 1H, CH), 11.42 (br, 1H, ArOH).

### Example 66: (2S)-Cyclopentylmethyl 2-(((4-formyl-5-hydroxy-6-methylpyridin-3-yl)methoxy)(phenoxy)phosphorylamino)propanoate (16x)

¹H NMR (299.992 MHz, chloroform-d) δ1.05-1.43 (m, 7H, CH, CH₂, CH₃), 1.44-1.81 (m, 9H, CH, CH₂), 2.04-2.26 (m, 1H, CH), 2.54 (s, 3H, ArCH₃), 3.47-3.70 (m, 1H, CH), 3.85-4.09 (m, 3H, NH, CH₂), 5.23-5.42 (m, 2H, CH₂), 7.06-7.20 (m, 3H, ArH), 7.20-7.35 (m, 3H, ArH), 8.02-8.14 (m, 1H, ArH), 10.26-10.36 (m, 1H, CHO), 11.44-11.53 (m, 1H, ArOH).

### Example 67: Isopropyl 4,4,4-trifluoro-2-(((4-formyl-5-hydroxy-6-methylpyridin-3-yl)methoxy)(phenoxy)phosphorylamino)butanoate (16xi)

¹H NMR (299.992 MHz, chloroform-d) δ1.09-1.33 (m, 6H, 2CH₃), 2.41-2.73 (m, 5H, CH₂, ArCH₃), 3.68-3.95 (m, 1H, CH), 4.08-4.32 (m, 1H, NH), 4.91-5.12 (m, 1H, CH), 5.23-5.47 (m, 2H, CH₂), 7.05-7.21 (m, 3H, ArH), 7.22-7.33 (m, 2H, ArH), 8.01-8.13 (m, 1H, ArH), 10.20-10.34 (m, 1H, CHO), 11.48 (s, 1H, ArOH).

### Example 68: (2S)-Isopropyl 2-((3,4-dichlorophenoxy)((4-formyl-5-hydroxy-6-methylpyridin-3-yl)methoxy)phosphorylamino)propanoate (16xii)

¹H NMR (299.992 MHz, chloroform-d) δ 1.15-1.30 (m, 9H, 3CH₃), 2.56 (s, 1H, CH₃), 3.54-3.71 (m, 1H, CH), 3.80-4.00 (m, 1H, CH), 4.95-5.07 (m, 1H, NH), 5.31-5.43 (m, 2H, CH₂), 6.98-7.08 (m, 1H, ArH), 7.31-7.39 (m, 1H, ArH), 8.07-8.13 (m, 1H, ArH), 10.32-10.38 (m, 1H, CHO), 11.49 (s, 1H, OH).

### Example 69: Tert-butyl 3-((2S)-2-(((4-formyl-5-hydroxy-6-methylpyridin-3-yl)methoxy)(phenoxy)phosphorylamino)-3-isopropoxy-3-oxopropyl)-1H-indole-1-carboxylate (16xiii)

¹H NMR (299.992 MHz, chloroform-d) δ1.09-1.26 (m, 6H, CH₃), 1.61-1.75 (m, 9H, 3CH₃), 2.48-2.66 (m, 3H, ArCH₃), 3.03-3.26 (m, 2H, CH₂), 3.72-4.01 (m, 1H, NH), 4.15-4.41 (m, 1H, CH), 4.89-5.11 (m, 1H, CH), 5.12-5.40 (m, 2H, CH₂), 6.99-7.39 (m, 7H, ArH), 7.39-7.56 (m, 2H, ArH), 7.94-8.19 (m, 2H, ArH), 10.14-10.31 (m, 1H, CHO), 11.42-11.58 (m, 1H, ArOH).

### Example 70: (2S)-Naphthalen-2-yl 2-(((4-formyl-5-hydroxy-6-methylpyridin-3-yl)methoxy)(phenoxy)phosphorylamino)propanoate (16xiv)

¹H NMR (299.992 MHz, dimethyl sulfoxide-d₆/methanol-d₄) δ1.46-1.64 (m, 3H, CH₃), 2.55 (s, 3H, ArCH₃), 4.30-4.48 (m, 2H, CH, NH), 4.91-5.23 (m, 2H, 2CH), 6.53-6.59 (m, 1H, CH), 6.96-7.71 (m, 12H, ArH), 8.17 (s, 1H, ArH).

### Example 71: Isopropyl 2-(((4-formyl-5-hydroxy-6-methylpyridin-3-yl)methoxy)(phenoxy)phosphorylamino)acetate (16xv)

¹H NMR (299.992 MHz, dimethyl sulfoxide-d₆/methanol-d₄) δ1.17-1.28 (m, 6H, 2CH₃), 2.59 (s, 3H, ArCH₃), 3.70 (s, 2H, CH₂), NH), 4.93-5.22 (m, 3H, CH₂, CH), 6.58-6.63 (m, 1H, CH), 7.03-7.38 (m, 5H, ArH), 8.27 (s, 1H, ArOH).

### Example 72: (2S)-Isopropyl 2-(((4-formyl-5-hydroxy-6-methylpyridin-3-yl)methoxy)(5,6,7,8-tetrahydronaphthalen-1-yloxy)phosphorylamino)propanoate (16xvi)

¹H NMR (299.992 MHz, chloroform-d) δ1.44-1.50 (m, 6H, 2CH₃), 1.57-1.66 (m, 3H, CH₃), 1.88-2.01 (m, 4H, CH₂), 2.78 (s, 3H, CH₃), 2.80-3.01 (m, 4H, 2CH₂), 3.83-4.02 (m, 1H, CH), 4.10-4.28 (m, 1H, CH), 5.17-5.32 (m, 1H, NH), 5.44-5.66 (m, 2H, CH₂), 7.05-7.13 (m, 1H, ArH), 7.14-7.23 (m, 1H, ArH), 7.24-7.32 (m, 1H, ArH), 8.26-8.34 (m, 1H, ArH), 10.43-10.52 (m, 1H, CHO), 11.71 (s, 1H, OH).

### Example 73: (2S,2'S)-Isopropyl 2,2′-(((4-formyl-5-hydroxy-6-methylpyridin-3-yl)methoxy)phosphoryl)bis(azanediyl)dipropanoate (16xvii)

¹H NMR (299.992 MHz, chloroform-d) δ0.98 1.41 (m, 18H, 6CH₃), 2.57 (s, 3H, ArCH₃), 3.39-3.96 (m, 4H, 2CH, 2NH), 4.76-5.32 (m, 4H, CH₂, 2CH), 8.05 (s, 1H, ArH), 10.39 (s, 1H, CH), 11.25-11.65 (br, 1H, ArOH).

### Example 74: (2S)-Isopropyl 2-(((5-(dimethylcarbamoyloxy)-4-formyl-6-methylpyridin-3-yl)methoxy)(phenoxy)phosphorylamino)propanoate (16xviii)

¹H NMR (299.992 MHz, chloroform-d) δ1.20-1.27 (m, 6H, CH₃), 1.36-1.41 (m, 3H, CH₃), 2.52 (s, 3H, ArCH₃), 3.05 (s, 3H, NCH₃), 3.20 (s, 3H, NCH₃), 3.61-3.70 (m, 1H, CH), 3.93-4.08 (m, 1H, CH), 4.97-5.04 (m, 1H, CH), 5.44-5.50 (m, 2H, CH₂), 7.14-7.35 (m, 6H, ArH), 8.59-8.61 (m, 1H, ArH), 10.26-10.28 (m, 1H, CHO).

### Example 75: (2R)-Isopropyl 2-(((4-((E)-(4-bromo-3-fluorophenylimino)methyl)-5-hydroxy-6-methylpyridin-3-yl)methoxy)(phenoxy)phosphorylamino)propanoate (16xix)

¹H NMR (299.992 MHz, chloroform-d) δ1.10-1.47 (m, 9H, 3CH₃), 2.49-2.74 (m, 3H, ArCH₃), 3.60-4.12 (m, 2H, CH, NH), 4.93-5.51 (m, 3H, CH₂, CH), 6.99-7.44 (m, 7H, ArH), 7.57-7.71 (m, 1H, ArH), 8.01-8.20 (m, 1H, ArH), 9.10-9.28 (m, 1H, CH), 13.72 (br, 1H, ArOH).

### Example 76: Pyridoxal Prodrug 16 with a Deuterated Benzene Ring (16xx)

¹H NMR (299.992 MHz, chloroform-d) δ1.15-1.29 (m, 6H, 2CH₃), 1.29-1.39 (m, 3H, CH₃), 2.54 (s, 3H, ArCH₃), 3.50-3.72 (m, 1H, NH), 3.83-4.04 (m, 1H, CH), 4.91-5.04 (m, 1H, CH), 5.25-5.43 (m, 2H, CH₂), 8.10 (s, 1H, ArH), 10.27-10.36 (m, 1H, CHO), 11.49 (br, 1H, ArOH).

### Example 77: Pyridoxal Prodrug 16 with a Deuterated Isopropyl Group (16xxi)

¹H NMR (299.992 MHz, chloroform-d) δ1.39-1.52 (m, 3H, CH₃), 2.65 (s, 3H, ArCH₃), 3.74-3.96 (m, 1H, NH), 3.96-4.15 (m, 1H, CH), 5.41-5.43 (m, 2H, CH₂), 7.18-7.31 (m, 3H, ArH), 7.33-7.44 (m, 2H, ArH), 8.20 (s, 1H, ArH), 10.37-10.47 (m, 1H, CHO), 11.60 (br, 1H, ArOH).

### Example 78: Pyridoxal Prodrug 16 with Deuterated Methyl Groups of the Isopropyl Group (16xxii)

¹H NMR (299.992 MHz, chloroform-d) δ1.42-1.52 (m, 3H, CH₃), 2.54 (s, 3H, ArCH₃), 3.49-3.70 (m, 1H, CH), 3.82-4.02 (m, 1H, NH), 4.04-4.16 (m, 1H, CH), 5.25-5.46 (m, 2H, CH₂), 7.08-7.22 (m, 3H, ArH), 7.27-7.42 (m, 2H, ArH), 8.10 (s, 1H, ArH), 10.28-10.37 (m, 1H, CH), 11.49 (br, 1H, ArOH).

### Example 79: (2S)-p-Tolyl 2-(((4-formyl-5-hydroxy-6-methylpyridin-3-yl)methoxy)(phenoxy)phosphorylamino)propanoate (16xxiii)

¹H NMR (299.992 MHz, chloroform-d) δ1.68-1.57 (m, 3H, CH₃), 2.67-2.38 (m, 6H, 2 ArCH₃), 4.44-3.63 (m, 2H, NH, CH), 5.54-5.39 (m, 2H, CH₂), 7.02-7.88 (m, 2H, ArH), 7.45-7.19 (m, 7H, ArH), 8.20-8.17 (m, 1H, ArH), 10.46-10.37 (m, 1H, CHO), 11.61 (br s, 1H, ArOH).

### Example 80: (2S)-Cyclopentyl 2-(((4-formyl-5-hydroxy-6-methylpyridin-3-yl)methoxy)(phenoxy)phosphorylamino)propanoate (16xxiv)

¹H NMR (299.992 MHz, chloroform-d) δ1.37-1.28 (m, 3H, CH₃), 1.92-1.47 (m, 8H, 4CH₂), 2.54 (s, 3H, ArCH₃), 4.15-3.53 (m, 2H, NH, CH), 5.43-5.09 (m, 3H, CH, CH₂), 7.34-7.09 (m, 5H, ArH), 8.13-7.96 (m, 1H, ArH), 10.34-10.30 (m, 1H, CHO), 11.49 (br s, 1H, ArOH).

### Example 81: (2S)-Isopropyl 2-((2,3-dihydro-1H-inden-5-yloxy)((4-formyl-5-hydroxy-6-methylpyridin-3-yl)methoxy)phosphorylamino)propanoate (16xxv)

¹H NMR (299.992 MHz, chloroform-d) δ1.18-1.26 (m, 6H, 2CH₃), 1.32-1.39 (m, 3H, CH₃), 2.01-2.13 (m, 2H, CH₂), 2.54 (s, 3H, CH₃), 2.79-2.88 (m, 4H, 2CH₂), 3.52-3.71 (m, 1H, CH), 3.84-4.03 (m, 1H, NH), 4.93-5.07 (m, 1H, CH), 5.22-5.42 (m, 2H, CH₂), 6.80-6.89 (m, 1H, ArH), 6.94-7.00 (m, 1H, ArH), 7.03-7.10 (m, 1H, ArH), 8.05-8.12 (m, 1H, ArH), 10.23-10.32 (m, 1H, CHO), 11.49 (s, 1H, OH).

### Example 82: (2S)-Isopropyl 2-(((4-formyl-5-hydroxy-6-methylpyridin-3-yl)methoxy)(4-(trifluoromethoxy)phenoxy)phosphorylamino)propanoate (16xxvi)

¹H NMR (299.992 MHz, chloroform-d) δ1.26-1.35 (m, 6H, 2CH₃), 1.41-1.49 (m, 3H, CH₃), 2.65 (s, 3H, CH₃), 3.68-3.85 (m, 1H, CH), 3.92-4.11 (m, 1H, CH), 4.97-5.16 (m, 1H, NH), 5.42-5.51 (m, 2H, CH₂), 7.18-7.33 (m, 4H, ArH), 8.16-8.25 (m, 1H, ArH), 10.39-10.49 (m, 1H, CHO), 11.04-11.99 (br, 1H, OH).

### Example 83: Pyridoxal Prodrug 16 with Deuterated Methylene Group (16xxvii)

¹H NMR (299.992 MHz, chloroform-d) δ1.13-1.28 (m, 6H, 2CH₃), 1.29-1.40 (m, 3H, CH₃), 2.54 (s, 3H, ArCH₃), 3.51-3.75 (m, 1H, CH), 3.82-4.02 (m, 1H, NH), 3.77-4.16 (m, 1H, CH), 7.09-7.19 (m, 3H, ArH), 7.22-7.34 (m, 2H, ArH), 8.03-8.12 (m, 1H, ArH), 10.24-10.36 (m, 1H, CH), 11.50 (br, 1H, ArOH).

### Example 84: (2S)-Isopropyl 2-cyclopentyl-2-(((4-formyl-5-hydroxy-6-methylpyridin-3-yl)methoxy)(phenoxy)phosphorylamino)acetate (16xxviii)

¹H NMR (299.992 MHz, chloroform-d) δ1.18-1.31 (m, 6H, 2CH₃), 1.32-1.83 (m, 9H, CH, CH₂), 2.14-2.32 (m, 1H, CH), 2.63 (s, 3H, CH₃), 3.75-4.05 (m, 2H, NH, CH), 4.95-5.13 (m, 1H, CH), 5.33-5.52 (m, 5H, CH₂), 7.15-7.29 (m, 3H, ArH), 7.30-7.43 (m, 2H, ArH), 8.12-8.22 (m, 1H, ArH), 10.33-10.42 (m, 1H, CHO), 10.59 (brs, 1H, ArOH).

### Example 85: (2S)-Isopropyl 3-(4-(benzyloxy)phenyl)-2-(((4-formyl-5-hydroxy-6-methylpyridin-3-yl)methoxy)(phenoxy)phosphorylamino)propanoate (16xxix)

¹H NMR (299.992 MHz, chloroform-d) δ1.07-1.22 (m, 6H, 2CH₃), 2.53 (s, 3H, CH₃), 2.85-2.98 (m, 2H, CH₂), 3.29-3.60 (m, 3H, CH, CH₂), 4.02-4.23 (m, 2H, CH, NH), 4.85-5.33 (m, 6H, CH, CH₂), 6.75-6.92 (m, 2H, ArH), 6.94-7.19 (m, 5H, ArH), 7.19-7.46 (m, 7H, ArH), 7.96-8.08 (m, 1H, ArH), 10.16-10.26 (m, 1H, CHO), 11.40-11.57 (m, 1H, ArOH).

### Example 86: (2S)-Isopropyl 2-(((4-formyl-5-hydroxy-6-methylpyridin-3-yl)methoxy)(phenoxy)phosphorylamino)-3-(4-hydroxyphenyl)propanoate (16xxx)

¹H NMR (299.992 MHz, chloroform-d) δ1.05-1.24 (m, 6H, 2CH₃), 2.50 (s, 3H, ArCH₃), 2.74-3.04 (m, 2H, CH₂), 3.56-3.78 (m, 1H, CH), 3.91-4.26 (m, 1H, NH), 4.72-5.28 (m, 3H, CH, CH₂), 6.57-6.76 (m, 2H, ArH), 6.87-7.02 (m, 2H, ArH), 7.02-7.18 (m, 3H, ArH), 7.19-7.32 (m, 2H, ArH), 7.88-8.00 (m, 1H, ArH), 10.07-10.20 (m, 1H, CHO), 11.44 (br, 1H, OH).

### Example 87: (2S)-1-Methylpiperidin-4-yl 2-(((4-formyl-5-hydroxy-6-methylpyridin-3-yl)methoxy)(phenoxy)phosphorylamino)propanoate (16xxxi)

¹H NMR (299.992 MHz, methanol-d₄) δ1.41-1.25 (m, 3H, CH₃), 2.01-1.69 (m, 4H, 2CH₂), 2.53-3.39 (m, 6H, 2CH₂), 2.9-2.57 (m, 4H, 2CH₂), 4.03-3.88 (m, 1H, CH), 4.98-4.75 (m, 1H, CH), 5.28-5.18 (m, 1H, CH₂), 5.51-5.41 (m, 1H, CH₂), 7.40-7.14 (m, 5H, ArH), 7.96-7.87 (m, 1H, ArH), 10.45-10.40 (m, 1H, CHO).

### Example 88: (2S)-2-Methoxyethyl 2-(((4-formyl-5-hydroxy-6-methylpyridin-3-yl)methoxy)(phenoxy)phosphorylamino)propanoate (16xxxii)

¹H NMR (299.992 MHz, chloroform-d) 1.38 (m, 3H, CH₃), 2.54 (s, 3H, ArCH₃), 3.33 (s, 3H, OCH₃), 3.59-3.50 (m, 2H, CH₂), 3.95-4.11 (m, 1H, CH), 4.17-4.22 (m, 2H, CH₂), 5.37-5.28 (m, 2H, CH₂), 7.11-7.28 (m, 5H, ArH), 8.20 (s, 1H, ArH), 10.32 (s, 1H), 11.49 (s, 1H).

### Example 89: (2S)-Methyl 1-(((4-formyl-5-hydroxy-6-methylpyridin-3-yl)methoxy)(phenoxy)phosphoryl)pyrrolidine-2-carboxylate (16xxxiii)

¹H NMR (299.992 MHz, chloroform-d) δ1.75-2.21 (m, 4H, Proline), 2.54 (s, 3H, ArCH₃), 3.18-3.52 (m, 3H. Proline), 3.58-3.76 (m, 3H, COOCH₃), 4.18-4.42 (m, 1H, CH), 5.24-5.59 (m, 3H, CH₂, NH), 7.04-7.21 (m, 3H, ArH), 7.22-7.34 (m, 2H, ArH), 8.05-8.19 (m, 1H, ArH), 10.24-10.42 (m, 1H, CHO), 11.46-11.56 (m, 1H, ArOH)

### Example 90: (2S)-5,6,7,8-Tetrahydronaphthalen-2-yl 2-(((4-formyl-5-hydroxy-6-methylpyridin-3-yl)methoxy)(phenoxy)phosphorylamino)propanoate (16xxxiv)

¹H NMR (299.992 MHz, chloroform-d) δ1.49-1.56 (s, 3H, CH₃), 1.74-1.81 (m, 4H, 2CH₂), 2.52-2.55 (m, 3H, CH₃), 2.69-2.77 (m, 4H, 2CH₂), 4.07-4.15 (m, 2H, NH, CH), 5.34-5.43 (m, 2H, CH₂), 6.65-6.71 (m, 2H, ArH), 6.77-6.85 (m, 1H, ArH), 6.98-7.04 (m, 1H, ArH), 7.08-7.22 (m, 4H, ArH), 7.99-8.19 (m, 1H, ArH), 10.24-10.38 (m, 1H, ArH), 11.40-11.54 (m, 1H, CHO).

### Example 91: Pyridoxal Prodrug 16 with Deuterated Methylene and Methyl Groups (16xxxv)

¹H NMR (299.992 MHz, chloroform-d) δ1.05-1.28 (m, 6H, 2CH₃), 1.27-1.41 (m, 3H, CH₃), 3.55-3.77 (m, 1H, CH), 3.82-4.02 (m, 1H, NH), 4.04-4.16 (m, 1H, CH), 4.88-5.05 (m, 2H, CH), 7.08-7.22 (m, 3H, ArH), 7.27-7.42 (m, 2H, ArH), 8.05-8.13 (s, 1H, ArH), 11.49 (br, 1H, ArOH).

### Example 92: (S)-Isopropyl 2-((S)-((4-((E)-(cyclopentylimino)methyl)-5-hydroxy-6-methylpyridin-3-yl)methoxy)(phenoxy)phosphorylamino)propanoate (16xxxvi)

¹H NMR (299.992 MHz, chloroform-d) δ1.19-1.49 (m, 9H, 3CH₃), 1.67-2.16 (M, 8H, 4CH₂), 2.63 (s, 3H, ArCH₃), 3.69-4.13 (m, 3H, 2CH, NH), 5.00-5.15 (m, 1H, CH), 5.23-5.39 (m, 2H, CH₂), 7.18-7.43 (m, 5H, ArH), 8.04 (S, 1H, ArH), 8.83 (S, 1H, CH).

### Example 93: (2S)-Isopropyl 2-(((4-formyl-5-hydroxy-6-methylpyridin-3-yl)methoxy)(p-tolyloxy)phosphorylamino)propanoate (16xxxvii)

¹H NMR (299.992 MHz, chloroform-d) δ1.25-1.17 (m, 6H, 2CH₃), 1.38-1.30 (m, 3H, CH₃), 2.30 (m, 3H, ArCH₃), 2.54 (s, 3H, ArCH₃), 4.03-3.53 (m, 2H, NH, CH), 5.05-4.93 (m, 1H, CH), 5.41-5.26 (m, 2H, CH₂), 7.09-6.96 (m, 4H, ArH), 8.11-8.06 (m, 1H, ArH), 10.33-10.27 (m, 1H, CHO), 11.49 (s, 1H, ArOH).

### Example 94: (2S)-Benzyl 2-(((4-((E)-(4-cyanophenethylimino)methyl)-5-hydroxy-6-methylpyridin-3-yl)methoxy)(phenoxy)phosphorylamino)propanoate (16xxxviii)

¹H NMR (299.992 MHz, chloroform-d) δ1.37-1.50 (m, 3H, CH₃), 2.57-2.67 (m, 3H, ArCH₃), 3.07-3.19 (m, 2H, CH₂), 3.61-4.23 (m, 4H, CH, NH, CH₂), 5.09-5.34 (m, 4H, 2CH₂), 7.09-7.54 (m, 12H, ArH), 7.58-7.75 (m, 2H, ArH), 7.94-8.10 (m, 1H, ArH), 8.68-8.89 (m, 1H, ArH), 13.91 (br, 1H, ArOH).

### Example 95: (2S)-Isopropyl 2-((4-bromo-2-fluorophenoxy)((4-formyl-5-hydroxy-6-methylpyridin-3-yl)methoxy)phosphorylamino)propanoate (16xxxix)

¹H NMR (299.992 MHz, Chloroform-d) δ1.40-1.17 (m, 9H, 3CH₃), 2.57-2.53 (m, 3H, ArCH₃), 4.03-3.67 (m, 2H, NH, CH), 5.06-4.92 (m, 1H, CH), 5.44-5.34 (m, 2H, CH₂), 7.30-7.15 (m, 3H, ArH), 8.13-8.06 (m, 1H, ArH), 10.40-10.33 (m, 1H, CHO), 11.51 (s, 1H, ArOH).

### Example 96: (2S)-4-Chlorophenyl 2-(((4-formyl-5-hydroxy-6-methylpyridin-3-yl)methoxy)(phenoxy)phosphorylamino)propanoate (16xi)

¹H NMR (299.992 MHz, dimethyl sulfoxide-d₆/methanol-d₄) δ1.33-1.46 (m, 3H, CH₃), 2.58-2.66 (br, 3H, ArCH₃), 3.87-3.99 (m, 1H, CH), 5.14-5.34 (m, 2H, CH₂), 6.09 & 8.27 (s & s, 1H, CHO), 6.71-6.80 (m, 2H, ArH), 7.08-7.23 (m, 6H, ArH), 7.29-7.39 (m, 2H, ArH), 8.12-8.18 (m, 1H, ArH).

### Example 97: P5P analog 16: single ascending dose study (FIGS. 3-5)

### Method

Formulation—P5P analog 16 was formulated in 1% methylcellulose in water on the day of dosing.

Animals and group—17 CD ♂ rats with an approx. weigh of ˜250 g were used. Animals were split into 4 groups+1 extra (n=4 for each group). Animals were allowed a 5-day acclimatization period in animal facility. The animal room environment was controlled (temperature 22 f 0.2° C.; relative humidity 55 f 25%; 12 hours light/dark cycle, and 12 air changes per hour). A standard certified commercial rodent chow was provided to the animals ad libitum. Procedures involving the care and use of animals in this study were reviewed and approved by the Institutional Animal Care and Use Committee (IACUC) prior to conduct. One day prior to dosing on D-1, Rats were divided in 4 groups (Table 6). Animals were identified and bled for pre-dose blood. Rats in all groups were fasted overnight (12-14 hours) with access of water.

On day 0, animals in groups 1-2-3 were weighed, and dosed by oral gavage as described in Table 6. Each remaining dosing solutions were stirred (or slightly vortexed), a 50 ul sample were collected and placed in separate tube and both vials immediately frozen on dry ice. Food tray were returned to animals two hours post dosing.

Cage-side observation were done upon dosing, at the end of day, and 24 hrs post dosing (record adverse effect if observed). Animal were weighed again at 24 hrs post dosing.

TABLE 6
Groups of rats in single ascending dose study

| | | | | Dose vol. | Dose |
|---|---|---|---|---|---|
| Group | Compound | n | Route | (mL/kg) | (mg/kg) |
| 1 | 16 | 4 | PO | 10 | 10 |
| 2 | 16 | 4 | PO | 10 | 30 |
| 3 | 16 | 4 | PO | 10 | 100 |
| 4 | 16 | 4 | PO | 10 | 300 |

Blood was collected at indicated time points for all groups at: Pre-dose, 15′, 30′, 1 h, 2 h, 4 h, 6 h, 8 h, 24 h.

About 150 μL of blood was collected at indicated time points below using K₂EDTA tubes. Blood was kept on ice until centrifugation at 4° C. at 6000 RCF for 10 min. Plasma was collected and kept on dry ice until transferred at −80° C.

### Results

1. Oral administration—When dosed by oral gavage at 10 mg/kg, 30 mg/kg, 100 mg/kg and 300 mg/kg both the Cₘₐₓ and exposure (AUC) of the P5P plasma profile increased with increasing dose. Cₘₐₓ values associated with these dosed were: 290, 454, 2105 and 6621 ng/ml respectively. The corresponding exposure values for these same doses were: 3673, 6853, 38288 and 86198 ng/ml*hr. When plotted against dose, these Cₘₐₓ and exposure values were linearly related with R2 values greater than 0.99.

2. There were no adverse reactions seen in any of the animals at any dose tested.

### Example 98: P5P Analog 16: Pharmacokinetic Study 1 (FIGS. 6-8)

### Method

Formulation—Both P5P (pyridoxal-5-phosphate) and P5P analog 16 were formulated in phosphate buffered saline (PBS) as homogenous solutions on the day of dosing.

Animals and group—13 CD ♂ rats with an approx. weigh of ˜250 g were used. Animals were split into 4 groups+1 extra (n=3 for each group). Animals were allowed a 5-day acclimatization period in animal facility. The animal room environment was controlled (temperature 22 f 0.2° C.; relative humidity 55 f 25%; 12 hours light/dark cycle, and 12 air changes per hour). A standard certified commercial rodent chow was provided to the animals ad libitum. Procedures involving the care and use of animals in this study were reviewed and approved by the Institutional Animal Care and Use Committee (IACUC) prior to conduct. One day prior to dosing on D-1, Rats were divided in 4 groups (Table 7). Rats in Group 3 and 4 were fasted overnight (12-14 hours) with access to water.

On day 0, animals were identified, weighted, and dosed by intravenous (IV) injection or oral gavage (PO) as described in Table 7. Food tray were returned to Group 3 and 4, 2 hrs post dosing.

TABLE 7
Groups and treatment in pharmacokinetic study 1.

| | | | | Dose Vol | Dose |
|---|---|---|---|---|---|
| Group | Compound | n | Route | (mL/Kg) | (mg/kg) |
| 1 | P5P | 3 | IV | 1 | 1 |
| 2 | 16 | 3 | IV | 1 | 1 |
| 3 | P5P | 3 | PO | 10 | 10 |
| 4 | 16 | 3 | PO | 10 | 10 |

Blood was collected at indicated time points below for all groups: Pre-dose, 5′, 15′, 30′, 1 h, 2 h, 4 h, 6 h, 8 h, 24 h.

About 150 μL of blood was collected at indicated time points below using K₂EDTA tubes. Blood was kept on ice until centrifugation at 4° C. at 6000 RCF for 10 min. Plasma was collected and kept on dry ice until transferred at −80° C.

### Results

1. Intravenous (IV) dosing—When dosed IV at 1 mg/kg, the plasma concentration-time curve demonstrated that P5P had a Cₘₐₓ value of 3373 ng/ml occurring at 0.1 hours after dosing. The plasma P5P was cleared relatively quickly with essentially no compound left after 4 hours. The overall exposure of P5P was approximately 2200 ng/ml*hr. A small amount of pyridoxal (above baseline values) was detected in the plasma with a total exposure of 770 ng/ml*hr.

With IV administration, P5P analog 16 showed a smaller Cₘₐₓ value of 1019 ng/ml and was also cleared rapidly with the prodrug no longer detectable in the plasma after 6 hours. Both P5P and pyridoxal were detected in the plasma with both compounds showing a similar Cₘₐₓ value of approximately 200 ng/ml. Further, the P5P levels were somewhat sustained.

2. Oral administration—When dosed by oral gavage at 10 mg/kg, P5P was not detected in the plasma over the 24-hour assessment period. A large spike in pyridoxal was seen in the plasma having a Cₘₐₓ value of 3668 ng/ml.

Upon oral dosing of P5P analog 16, the P5P analog 16 was detectable in the plasma and showed a Cₘₐₓ value of 1345 ng/ml with the peak occurring at 0.5 hours after dosing. In addition, significant P5P concentrations were detected in the plasma. The P5P plasma profile after P5P analog 16 oral dosing showed a Cmax value of 445 ng/ml occurring at 1 hour post dose. This P5P plasma profile showed a half-life of approximately 7 hours. Pyridoxal showed a similar plasma profile.

### Example 99: P5P Analogs 16 and 16v.: Pharmacokinetic Study 2 (FIGS. 9—

### Method

Formulation—Both P5P analog 16 and P5P analog 16v were formulated in 40% hydroxypropyl-β-cyclodextrin in water as homogenous solutions on the day of dosing.

Animals and group—13 CD ♂ rats with an approx. weigh of ˜250 g were used. Animals were split into 4 groups+1 extra (n=3 for each group). Animals were allowed a 5-day acclimatization period in animal facility. The animal room environment was controlled (temperature 22 f 0.2° C.; relative humidity 55 f 25%; 12 hours light/dark cycle, and 12 air changes per hour). A standard certified commercial rodent chow was provided to the animals ad libitum. Procedures involving the care and use of animals in this study were reviewed and approved by the Institutional Animal Care and Use Committee (IACUC) prior to conduct. One day prior to dosing on D-1, Rats were divided in 4 groups (Table 8). Rats in Group 3 and 4 were fasted overnight (12-14 hours) with access to water.

On day 0, animals were identified, weighted, and dosed by intravenous (IV) injection or oral gavage (PO) as described in Table 8. Food tray were returned to Group 3 and 4, 2 hrs post dosing.

TABLE 8
Group and treatment in pharmacokinetic study 2.

| | | | | Dose vol. | Dose |
|---|---|---|---|---|---|
| Group | Compound | n | Route | (mL/kg) | (mg/kg) |
| 1 | 16v | 3 | IV | 1 | 1 |
| 2 | 16 | 3 | IV | 1 | 1 |
| 3 | 16v | 3 | PO | 10 | 10 |
| 4 | 16 | 3 | PO | 10 | 10 |

Blood was collected at indicated time points below for all groups: Pre-dose, 5′, 15′, 30′, 1 h, 2 h, 4 h, 6 h, 8 h, 24 h.

About 150 μL of blood was collected at indicated time points below using K₂EDTA tubes. Blood was kept on ice until centrifugation at 4° C. at 6000 RCF for 10 min. Plasma was collected and kept on dry ice until transferred at −80° C.

### Results

Intravenous (IV) dosing—When dosed IV at 1 mg/kg, the plasma concentration-time curve for P5P analog 16v demonstrated a small Cₘₐₓ value of only 285 ng/ml occurring at 0.1 hours after dosing. Conversely, the plasma P5P profile showed that a large amount of P5P was released into the plasma after dosing the P5P analog. The PLP profile showed a Cₘₐₓ of 1162 ng/ml, a Tₘₐₓ of 0.3 hours and a half-life of 8.5 hours. A smaller amount of pyridoxal was also formed and detected in the plasma.

With IV administration, P5P analog 16 showed a similar profile to the previous study with similar amounts of P5P and pyridoxal being present in the plasma.

Oral administration—When dosed by oral gavage at 10 mg/kg, P5P analog 16v showed large differences in the amount of the P5P analog and P5P between the individual rats. On average there was very little P5P analog seen in the plasma after oral dosing (Cₘₐₓ=198 ng/ml). On the other hand, P5P showed a similar average plasma profile as P5P analog 16 with a Cₘₐₓ that was somewhat larger (329 ng/ml vs 238 ng/ml). The P5P exposure when given as P5P analog 16v was 3892 ng/ml*hr compared to 3042 ng/ml*hr when given as P5P analog 16. When looking at the individual rat data it can be seen that this larger value was due to a single rat.

The concentration-time curves for the oral dosing of P5P analog 16 showed lowed amounts of the P5P analog but similar amounts of P5P to the previous study.

### Example 100: P5P Analog 16vi and Compound II.I.: Pharmacokinetic Study 3 (FIGS. 11-13)

Compound II.I. is a P5P analog not encompassed by the compound of Formula I.

### Method

Formulation—Compound II.I. was formulated in 0.9% saline while P5P analog 16vi was formulated in 40% hydroxypropyl-β-cyclodextrin in water as a homogenous solution on the day of dosing.

Animals and group—13 CD ♂ rats with an approx. weigh of ˜250 g were used. Animals were split into 4 groups+1 extra (n=3 for each group). Animals were allowed a 5-day acclimatization period in animal facility. The animal room environment was controlled (temperature 22 f 0.2° C.; relative humidity 55 f 25%; 12 hours light/dark cycle, and 12 air changes per hour). A standard certified commercial rodent chow was provided to the animals ad libitum. Procedures involving the care and use of animals in this study were reviewed and approved by the Institutional Animal Care and Use Committee (IACUC) prior to conduct. One day prior to dosing on D-1, Rats were divided in 4 groups (Table 9). Rats in Group 3 and 4 were fasted overnight (12-14 hours) with access to water.

On day 0, animals were identified, weighted, and dosed by intravenous (IV) injection or oral gavage (PO) as described in Table 9. Food tray were returned to Group 3 and 4, 2 hrs post dosing.

TABLE 9
Groups and treatment in pharmacokinetic study 3.

| | | | | Dose vol. | Dose |
|---|---|---|---|---|---|
| Group | Compound | n | Route | (mL/Kg) | (mg/kg) |
| 1 | Compound II.I. | 3 | IV | 1 | 1 |
| 2 | 16vi | 3 | IV | 1 | 1 |
| 3 | Compound II.I. | 3 | PO | 10 | 10 |
| 4 | 16vi | 3 | PO | 10 | 10 |

Blood was collected at indicated time points below for all groups: Pre-dose, 5′, 15′, 30′, 1 h, 2 h, 4 h, 6 h, 8 h, 24 h.

About 150 μL of blood was collected at indicated time points below using K₂EDTA tubes. Blood was kept on ice until centrifugation at 4° C. at 6000 RCF for 10 min. Plasma was collected and kept on dry ice until transferred at −80° C.

### Results

Intravenous (IV) dosing—When Compound II.I. was dosed IV at 1 mg/kg, very little of Compound II.I. or P5P was detected in the plasma. The Compound II.I. had a Cₘₐₓ of 777 ng/ml and an overall exposure of 346 ng/ml*hr. The resultant P5P from dosing this prodrug had an exposure of 1534 ng/ml*hr.

IV dosing with the P5P analog 16vi showed a similar rapid clearance of the P5P analog 16vi but somewhat better P5P levels with an exposure of 3666 ng/ml*hr and a half-life of 10 hours.

Oral administration—When dosed by oral gavage at 10 mg/kg, Compound II.I. showed Compound II.I. and P5P profiles similar to P5P analog 16i. When P5P analog 16vi was dosed orally at 10 mg/kg, the P5P analog 16vi showed a Cₘₐₓ value of 1652 ng/ml and an exposure of 1592 ng/ml*hr. The resultant P5P plasma profile had a Cmax of 190 ng/ml, a half-life of 6.5 hours and an overall exposure of 1549 ng/ml*hr.

When dosed orally, P5P analog 16vi nor P5P were detected in the plasma at any significant levels.

### Example 101: P5P Analog 16vi and Compound II.I.: Pharmacokinetic Study 4 (FIGS. 14-17)

### Method

Formulation—Prodrugs 16i (S_{P} isomer), 16i (R_{P} isomer) and 16i′ (16i with D-amino acid racemate) were formulated in phosphate buffered saline (PBS) as a homogenous solution on the day of dosing.

Animals and group—19 CD ♂ rats with an approx. weigh of ˜250 g were used. Animals were split into 6 groups+1 extra (n=3 for each group). Animals were allowed a 5-day acclimatization period in animal facility. The animal room environment was controlled (temperature 22 f 0.2° C.; relative humidity 55 f 25%; 12 hours light/dark cycle, and 12 air changes per hour). A standard certified commercial rodent chow was provided to the animals ad libitum. Procedures involving the care and use of animals in this study were reviewed and approved by the Institutional Animal Care and Use Committee (IACUC) prior to conduct. One day prior to dosing on D-1, Rats were divided in 6 groups (Table 1). Animals were identified and bled for pre-dose blood. Rats in Group 4, 5 and 6 were fasted overnight (12-14 hours) with access of water.

On day 0, animals were weighted, and dosed by IV injection or by oral gavage as described in Table 10. Food tray were returned to Group 4, 5 and 6 two hours post dosing.

TABLE 10
Groups and treatment in pharmacokinetic study 4.

| | | | | dose | |
|---|---|---|---|---|---|
| Group | Compound | n | route | volume | dose |
| 1 | 16i (S, isomer) | 3 | IV | 1 mL/kg | 1 mg/kg |
| 2 | 16ii (R isomer) | 3 | IV | 1 mL/kg | 1 mg/kg |
| 3 | 16iii (racemic with D amino acid) | 3 | IV | 1 mL/kg | 1 mg/kg |
| 4 | 16i | 3 | PO | 10 mL/kg | 10 mg/kg |
| 5 | 16ii | 3 | PO | 10 mL/kg | 10 mg/kg |
| 6 | 16iii | 3 | PO | 10 mL/kg | 10 mg/kg |

Blood was collected at indicated time points below for all groups: Pre-dose, 5′, 15′, 30′, 1 h, 2 h, 4 h, 6 h, 8 h, 24 h.

About 150 μL of blood was collected at indicated time points below using K₂EDTA tubes. Blood was kept on ice until centrifugation at 4° C. at 6000 RCF for 10 min. Plasma was collected and kept on dry ice until transferred at −80° C.

### Results

Intravenous (IV) dosing—IV dosing of the 2 diastereomers of prodrug 16 (S_{P} and R_{P} isomers) resulted in similar conversion to PLP with the S_{P} isomer prodrug showing a slight advantage. When S_{P} isomer was given IV, the Cmax and exposure of PLP were 200 ng/ml and 2962 ng/ml*hr respectively. When R_{P} isomer was given IV, the Cmax and exposure values were 177 ng/ml and 1886 ng/ml*hr respectively.

The D-amino acid prodrug 16iii did not convert well to PLP when dosed IV showing a Cmax value of less than 100 ng/ml.

Oral administration—When dosed by oral gavage at 10 mg/kg, both diastereomers produced a near identical PLP plasma profiles. Comparing S_{P} isomer vs R_{P} isomer, Cmax values were 472 vs 409 ng/ml, half-life values were 7.6 vs 7 hours and exposures values were 5892 vs 4673 ng/ml*hr.

The amount of PLP detected in the plasma when animals were dosed with prodrug 16iii was small with an overall exposure of 645 ng/ml*hr.

Given the variability between animals, there was no significant difference between the two diastereomers of prodrug 16 (ie. prodrugs S_{P} and R_{P}). Further, the D-amino acid prodrug 16iii did not show a better PLP plasma profile compared to prodrug 16—the L-amino acid version of this prodrug.

### Plasma Conversion Assay

The ability of plasma enzymes to convert the various prodrugs into P5P was tested by incubating the prodrugs in either rat (Sprague Dawley) whole blood or plasma. Rat blood or plasma (Innovative Research Inc) was spiked with 10 mM (DMSO) of the prodrugs for a final concentration of 10 uM. The blood or plasma was then sampled at various time points and analyzed for the presence of P5P.

Blood (4 ml) was pipetted into a 15 ml Falcon tube and incubated in a 37 degree/5% CO₂ incubator for approximately 30 minutes before spiking with 10 mM (4 uL) prodrug. The Falcon tube was gently vortexed on low speed and the blood pipetted into 5 separate Eppendorf tubes (˜250 uL). At various time points (up to 6 hours), the Eppendorf tubes were centrifuged at 554 RCF and 50 μL of supernatant removed to a separate Eppendorf tube that was flash frozen and stored at −80° C. until analysis.

Plasma samples were treated in a similar way such that 1 ml of plasma was spiked (1 uL) with prodrug and vortexed before 50 ul was pipetted into Eppendorf tubes that were flash frozen at various time points. Plasma samples were also stored at −80° C. until analysis.

The peak amount of amount of P5P generated from the various prodrugs was normalized to the amount of P5P converted from 16 included in each experiment.

### Analysis (LC/MS)—LC-MS/MS Conditions for P5P Analysis

Instrument: BRUKER EVOQ
MS Conditions
ESI positive mode in MRM
Source temperature 120° C.
Desolvation temperature 400° C.
Capillary voltage 3500 V

| Compound | | |
|---|---|---|
| name | Parent(m/z) | Daughter(m/z) |
| P5P | 247.9 | 150.0 |

### LC Conditions

Mobile phase—A: 0.3% formic acid in water,
Mobile phase-β: 0.1% formic acid in acetonitrile
HPLC Column: ACQUITY UPLC HSS T3 1.8 μm, 2.1×100 mm column

### Gradient

| | Total time | Flow rate | | |
|---|---|---|---|---|
| Step | (min) | (mL/min) | A % | B % |
| 1 | Initial | 0.3 | 80 | 20 |
| 2 | 1.4 | 0.3 | 50 | 50 |
| 3 | 2 | 0.3 | 80 | 20 |
| 4 | 3 | 0.3 | 80 | 20 |

### Sample Preparation

Aliquot 50 μL of rat plasma sample, add 950 μL of trichloro acetic acid 50 g/L.
Vortex for 1 minute.
Centrifuge at 19000 RCF for 45 mins
Supernatant injected (20 μL) to LC-MS/MS for analysis.

Results of the ability of plasma enzymes to convert the various prodrugs into P5P, performed in separate tests, in comparison to 16 is provided in Table 11 below.

TABLE 11
Results of tests for conversion of various analogs
into P5P in comparison to 16i.

| | P5P formed | relative P5P |
|---|---|---|
| Compound | (uM) | formed (%) |
| 16i | 4.2 | 100.0 |
| 14 | 0 | 0.0 |
| 14i | 0.17 | 4.0 |
| 16i | 5.7 | 100.0 |
| 15i | 0.15 | 2.6 |
| 16xix | 2.3 | 40.4 |
| 16i | 4.3 | 100.0 |
| 15vi | 0 | 0.0 |
| 15x | 0 | 0.0 |
| 16i | 3.8 | 100.0 |
| 15xxxiv | 0 | 0.0 |
| 16iv | 1.8 | 47.4 |
| 16i | 2.6 | 100.0 |
| 15ii | 0 | 0.0 |
| 14ii | 0 | 0.0 |
| 16i | 1.6 | 100.0 |
| 15vii | 0 | 0.0 |
| 16x | 1.95 | 121.9 |
| 16i | 5.1 | 100.0 |
| 16xviii | 5.8 | 113.7 |
| 15xviii | 0.5 | 9.8 |
| 15xiii | 0.8 | 15.7 |
| 16vii | 3.6 | 70.6 |
| 16i | 10.5 | 100.0 |
| 14iii | 0.7 | 6.7 |
| 15xi | 1.1 | 10.5 |
| 16ix | 10.3 | 98.1 |
| 15iv | 0.65 | 6.2 |
| 16xiv | unstable in DMSO | — |
| 16i | 5.4 | 100.0 |
| 16viii | 5.4 | 100.0 |
| 16viii (2^{nd} sample) | 5.2 | 96.3 |
| 15iii | 0.4 | 7.4 |
| 16xv | unstable in DMSO | — |
| 15xix | 0.4 | 7.4 |
| 16i | 4.1 | 100.0 |
| 16xxii | 6.3 | 153.7 |
| 14vii | 0.5 | 12.2 |
| 16xxix | 2.4 | 58.5 |
| 16xxviii | 0.4 | 9.8 |
| 14ix | 0.4 | 6.3 |
| 16i | 3.6 | 100.0 |
| 15xvii | 0.3 | 8.3 |
| 14vi | 0.2 | 5.6 |
| 16xx | 1.4 | 38.9 |
| 14xiv | 0.3 | 8.3 |
| 14iv | 2 | 55.6 |
| 16i | 3.8 | 100.0 |
| 16vii | 0.4 | 10.5 |
| 14xii | 0.2 | 5.3 |
| 15xiv | 0.1 | 2.6 |
| 16xi | 0.6 | 15.8 |
| 16xiii | 0.3 | 7.9 |
| 16i | 2.7 | 100.0 |
| 15xii | 0.2 | 7.4 |
| 14iv | 0.3 | 11.1 |
| 14v | 0.3 | 11.1 |
| 16xxiii | 2 | 74.1 |
| 14x | 0.3 | 11.1 |
| 16i | 1.7 | 100.0 |
| 16xxiv | 2.2 | 129.4 |
| 14viii | 0.3 | 17.6 |
| 16xxi | 0.3 | 17.6 |
| 14xi | 0.3 | 17.6 |
| 14xiii | 0.3 | 17.6 |
| 16i | 2.9 | 100.0 |
| 16xxvii | 2 | 69.0 |
| 16xxx | 2.2 | 75.9 |
| 14xv | 0.3 | 10.3 |
| 14xvi | 0.3 | 10.3 |
| 16xxxi | 0.4 | 13.8 |
| 16i | 3.4 | 100.0 |
| 16xxxii | 4.9 | 144.1 |
| 16xxxiii | 0.4 | 11.8 |
| 16xxxiv | 4.9 | 144.1 |
| 16xxxviii | 0.5 | 14.7 |
| 14xvii | 6.6 | 194.1 |
| 16i | 2.5 | 100.0 |
| 15xxxi | 0.5 | 20.0 |
| 16xxxvi | 3.8 | 152.0 |
| 16xxxvii | 0.3 | 12.0 |
| 14xviii | 2.4 | 96.0 |
| 16xxxix | 2.3 | 92.0 |
| 16i | 2.1 | 100.0 |
| 16xL | 3.6 | 171.4 |
| 14iv | 0.3 | 14.3 |

Certain adaptations and modifications of the described embodiments can be made. Therefore, the above discussed embodiments are considered to be illustrative and not restrictive.

1. A compound, its stereoisomer, salt, hydrate, solvate, isotope or crystalline form thereof, represented by formula I: 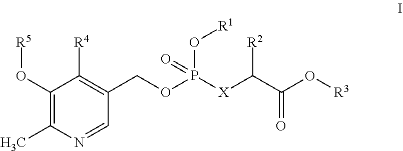
wherein
X is NH or N;
R¹ is H or a C₁₋₁₅ substituent having one or more heteroatoms;
R² is H or a C₁₋₁₅ substituent having one or more heteroatoms, or when X is N, R² and N form a cyclic structure having C₃₋₁₅ atoms having one or more heteroatoms;
R³ is H or a C₁₋₁₅ substituent having one or more heteroatoms;
R⁴ is 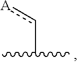
wherein [symbol] is a single or a double bond, and wherein
when [symbol] is a single bond,
A is NH₂, NH-PG_{N}, 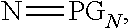
OH, O-PG_{O1}, where PG_{N} is a protecting group bonded to a nitrogen, and PG_{O1} is a first protecting group bonded to an oxygen;
when [symbol] is a double bond
A is O; and
R⁵ is H or PG_{O2}, wherein PG_{O2} is a second protecting group bonded to oxygen, or PG_{O1} and PG_{O2} together form a diol protecting group.

2. The compound, its stereoisomer, salt, hydrate, solvate, isotope or crystalline form thereof, represented by Formula I, as defined in claim 1, wherein:
(i) R¹ is —C₆H₅, —C₆H₄Cl, —C₆H₃BrF, —C₆H₃Cl₂, —C₆D₅, —C₇H₇, —C₇H₄OF₃, —C₇H₇O, —C₉H₉, —C₁₀H₁₁ or —C₁₀H₇;
(ii) R² is —H, -D, —CH₃, —C₂H₂F₃, —CH₂CH₂CH₂—, —C₄H₉, —C₅H₉, —C₆H₅, —C₇H₇, —C₇H₇O, —C₁₄H₁₂O or —C₁₄H₁₆NO₂;
(iii) R³, is —CH₃, —C₃H₇, —C₃HD₆, —C₃D₇, —C₅H₉, —C₆H₄Cl, —C₆H₇, —C₆H₁₁, —C₆H₁₂N, —C₆H₁₃, —C₇H₇, —C₁₀H₇ or —C₁₀H₁₁;
(iv) PG_{N} is fluorenylmethoxycarbonyl protecting group (Fmoc), tert-butoxycarbonyl (BOC), carbobenzyloxy (Cbz), trifluoroacetamide, phthalimide, trityl (Tr), monomethoxytrityl (MMT), dimethoxytrityl (DMT), or benzylideneamine;
(v) each PG_{O1} and PG_{O2} independently is benzyl (Bn), t-butyldimethylsilyl (TBDMS), t-butyldiphenylsilyl (TBDPS), acetyl (Ac), pivaloyl (Piv), or Benzyl (Bz), trityl (Tr), monomethoxytrityl (MMT), dimethoxytrityl (DMT), or PG_{O1} and PG_{O2} together form 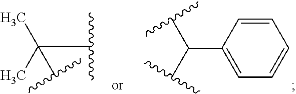
or
(vi) one or more of the H is replaced by D.

3-7. (canceled)

8. The compound as defined in claim 1, wherein the compound is: 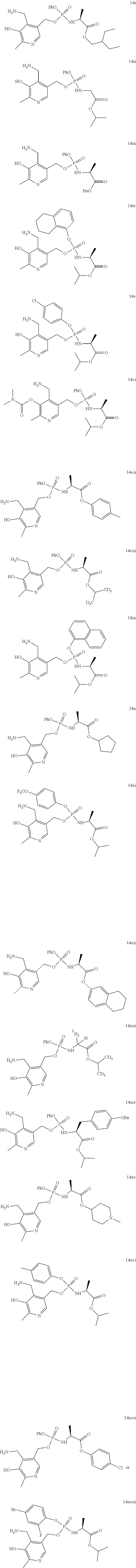
its stereoisomer, salt, hydrate, solvate, isotope or crystalline form thereof.

9. The compound as defined in claim 1, wherein the compound is: 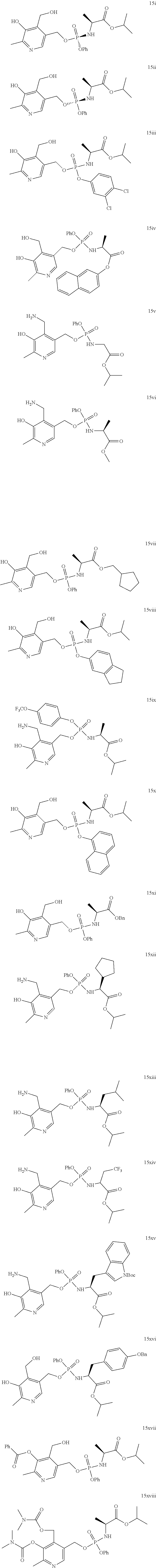 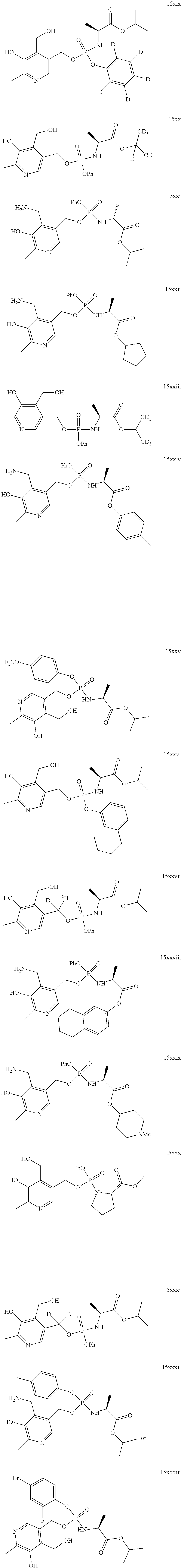
its stereoisomer, salt, hydrate, solvate, isotope or crystalline form thereof.

10. The compound as defined in claim 1, wherein the compound is: [chemical structure]
its stereoisomer, salt, hydrate, solvate, isotope or crystalline form thereof.

11. The compound as defined in claim 1, wherein the compound is of formula ea 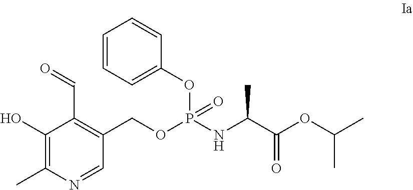 [SMILES: Cc1ncc(COP(=O)(N[C@@H](C)C(=O)OC(C)C)Oc2ccccc2)c(C=O)c1O]
its stereoisomer, salt, hydrate, solvate, isotope or crystalline form thereof.

12. A process for preparation of a compound, its stereoisomer, salt, hydrate, solvate, isotope or crystalline form thereof, of Formula I: 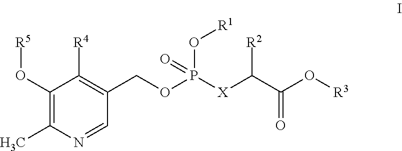
the process comprising the step of reacting the compound of Formula II with a compound of Formula III, in the presence of a base, to form the compound of Formula I 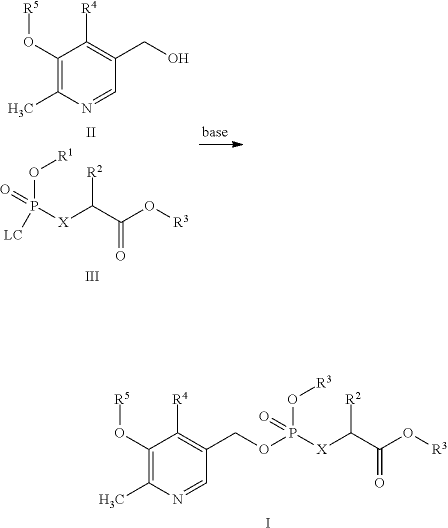
wherein
X is NH or N;
R¹ is H or a C₁₋₁₅ substituent having one or more heteroatoms;
R² is H or a C₁₋₁₅ substituent having one or more heteroatoms, or when X is N, R² and N form a cyclic structure having C₂₋₁₅ atoms having one or more heteroatoms;
R³ is H or a C₁₋₁₅ substituent having one or more heteroatoms;
LG is a leaving group;
R⁴ is 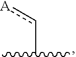
wherein [symbol] is a single or a double bond, and wherein
when [symbol] is a single bond,
A is NH₂, NH-PG_{N}, 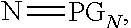
OH, O-PG_{O1}, where PG_{N} is a protecting group bonded to a nitrogen, and PG_{O1} is a first protecting group bonded to an oxygen;
when [symbol] is a double bond
A is O;
R⁵ is H or PG_{O2}, wherein PG_{O2} is a second protecting group bonded to oxygen, or PG_{O1} and PG_{O2} together form a diol protecting group.

13. The process as defined in claim 12,
(i) further comprising the step of deprotection to remove any protecting groups in the compound of Formula I; or
(ii) wherein when [symbol] is a single bond and A is OH, further comprising the step of oxidation of the compound to form a compound where [symbol] is a double bond and A is O.

14. (canceled)

15. The process as defined claim 12, further comprising the steps of: 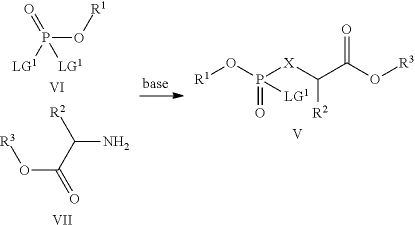
reacting the compound of Formula VII with a compound of Formula VI, in the presence of a base, to form the compound of Formula V,
wherein LG¹ is a leaving group, and is the same or different from the leaving group LG.

16. The process as defined in claim 15, wherein when LG¹ is different from LG, further comprising the step of: 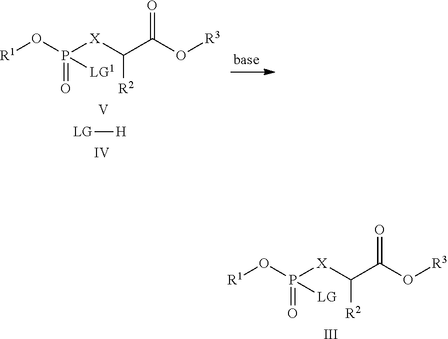
reacting the compound of Formula V with the compound of Formula IV, in the presence of a base, to form the compound of Formula III.

17. A composition comprising:
a carrier, diluent or excipient, and
a compound, its stereoisomer, salt, hydrate, solvate, isotope or crystalline form thereof, represented by formula I: 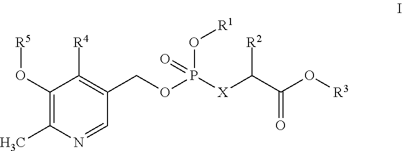
wherein
X is NH or N;
R¹ is H or a C₁₋₁₅ substituent having one or more heteroatoms;
R² is H or a C₁₋₁₅ substituent having one or more heteroatoms, or when X is N, R² and N form a cyclic structure having C₂₋₁₅ atoms having one or more heteroatoms;
R³ is H or a C₁₋₁₅ substituent having one or more heteroatoms;
R⁴ is 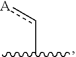
wherein [symbol] is a single or a double bond, and wherein
when [symbol] is a single bond,
A is NH₂, NH-PG_{N}, 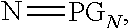
OH, O-PG_{O1}, where PG_{N} is a protecting group bonded to a nitrogen, and PG_{O1} is a first protecting group bonded to an oxygen;
when [symbol] is a double bond
A is O; and
R⁵ is H or PG_{O2}, wherein PG_{O2} is a second protecting group bonded to oxygen, or PG_{O1} and PG_{O2} together form a diol protecting group.

18. The composition of claim 17, wherein in the compound, its stereoisomer, salt, hydrate, solvate, isotope or crystalline form thereof, represented by Formula I,
(i) R¹ is —C₆H₅, —C₆H₄Cl, —C₆H₃BrF, —C₆H₃Cl₂, —C₆D₅, —C₇H₇, —C₇H₄OF₃, —C₇H₇O, —C₉H₉, —C₁₀H₁₁ or —C₁₀H₇;
(ii) R² is —H, -D, —CH₃, —C₂H₂F₃, —CH₂CH₂CH₂—, —C₄H₉, —C₅H₉, —C₆H₅, —C₇H₇, —C₇H₇O, —C₁₄H₂O or —C₁₄H₁₆NO₂;
(iii) R³ is —CH₃, —C₃H₇, —C₃HD₆, —C₃D₇, —C₅H₉, —C₆H₄Cl, —C₆H₇, —C₆H₁₁, —C₆H₁₂N, —C₆H₁₃, —C₇H₇, —C₁₀H₇ or —C₁₀H₁₁;
(iv) PG_{N} is fluorenylmethoxycarbonyl protecting group (Fmoc), tert-butoxycarbonyl (BOC), carbobenzyloxy (Cbz), trifluoroacetamide, phthalimide, trityl (Tr), monomethoxytrityl (MMT), dimethoxytrityl (DMT), or benzylideneamine;
(v) each PG_{O1} and PG_{O2} independently is benzyl (Bn), t-butyldimethylsilyl (TBDMS), t-butyldiphenylsilyl (TBDPS), acetyl (Ac), pivaloyl (Piv), or Benzyl (Bz), trityl (Tr), monomethoxytrityl (MMT), dimethoxytrityl (DMT), or
PG_{O1} and PG_{O2} together form 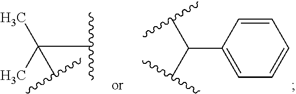
or
(vi) one or more of the H is replaced by D.

19-23. (canceled)

24. The composition of claim 17, wherein in the compound is 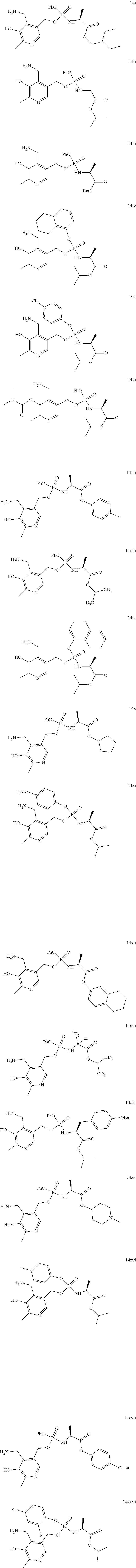
its stereoisomer, salt, hydrate, solvate, isotope or crystalline form thereof.

25. The composition of claim 17, wherein in the compound is 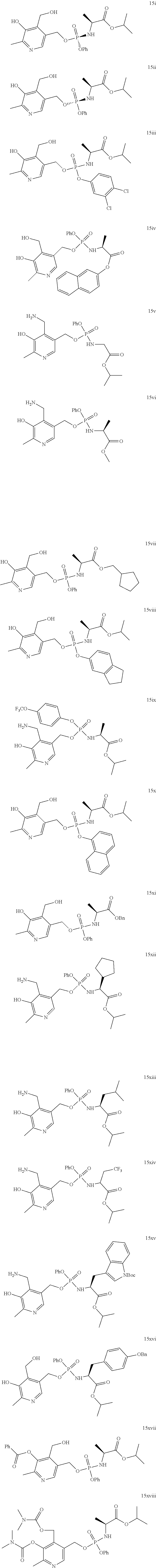 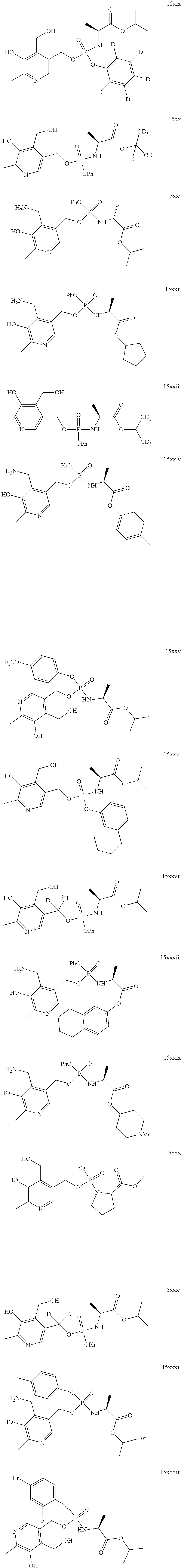
its stereoisomer, salt, hydrate, solvate, isotope or crystalline form thereof.

26. The composition of claim 17, wherein in the compound is [chemical structure]
its stereoisomer, salt, hydrate, solvate, isotope or crystalline form thereof.

27. The composition of claim 17, wherein in the compound is the compound of Formula Ia 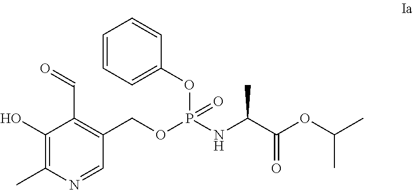 [SMILES: Cc1ncc(COP(=O)(N[C@@H](C)C(=O)OC(C)C)Oc2ccccc2)c(C=O)c1O]
its stereoisomer, salt, hydrate, solvate, isotope or crystalline form thereof.

28-38. (canceled)

39. A method of medical treatment or prophylaxis of a disease, comprising administering, to a subject in need thereof, a compound, its stereoisomer, salt, hydrate, solvate, isotope or crystalline form thereof, represented by formula I: 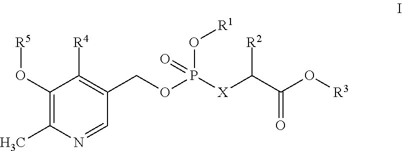
wherein
X is NH or N;
R¹ is H or a C₁₋₁₅ substituent having one or more heteroatoms;
R² is H or a C₁₋₁₅ substituent having one or more heteroatoms, or when X is N, R² and N form a cyclic structure having C₂₋₁₅ atoms having one or more heteroatoms;
R³ is H or a C₁₋₁₅ substituent having one or more heteroatoms;
R⁴ is 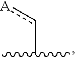
wherein [symbol] is a single or a double bond, and wherein
when [symbol] is a single bond,
A is NH₂, NH-PG_{N}, 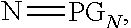
OH, O-PG_{O1}, where PG_{N} is a protecting group bonded to a nitrogen, and PG_{O1} is a first protecting group bonded to an oxygen;
when [symbol] is a double bond
A is O; and
R⁵ is H or PG_{O2}, wherein PG_{O2} is a second protecting group bonded to oxygen, or PG_{O1} and PG_{O2} together form a diol protecting group.

40. The method of claim 39, wherein in the compound, its stereoisomer, salt, hydrate, solvate, isotope or crystalline form thereof, represented by Formula I,
(i) R¹ is —C₆H₅, —C₆H₄Cl, —C₆H₃BrF, —C₆H₃Cl₂, —C₆D₅, —C₇H₇, —C₇H₄OF₃, —C₇H₇O, —C₉H₉, —C₁₀H₁₁ or —C₁₀H₇;
(ii) R² is —H, -D, —CH₃, —C₂H₂F₃, —CH₂CH₂CH₂—, —C₄H₉, —C₅H₉, —C₆H₅, —C₇H₇, —C₇H₇O, —C₁₄H₁₂O or —C₁₄H₁₆NO₂;
(iii) R³ is —CH₃, —C₃H₇, —C₃HD₆, —C₃D₇, —C₅H₉, —C₆H₄Cl, —C₆H₇, —C₆H₁₁, —C₆H₁₂N, —C₆H₁₃, —C₇H₇, —C₁₀H₇ or —C₁₀H₁₁;
(iv) PG_{N} is fluorenylmethoxycarbonyl protecting group (Fmoc), tert-butoxycarbonyl (BOC), carbobenzyloxy (Cbz), trifluoroacetamide, phthalimide, trityl (Tr), monomethoxytrityl (MMT), dimethoxytrityl (DMT), or benzylideneamine;
(v) each PG_{O1} and PG_{O2} independently is benzyl (Bn), t-butyldimethylsilyl (TBDMS), t-butyldiphenylsilyl (TBDPS), acetyl (Ac), pivaloyl (Piv), or Benzyl (Bz), trityl (Tr), monomethoxytrityl (MMT), dimethoxytrityl (DMT), or
PG_{O1} and PG_{O2} together form 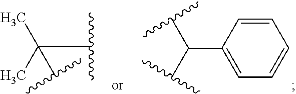
or
(vi) one or more of the H is replaced by D.

41-45. (canceled)

46. The method of claim 39, wherein the compound is 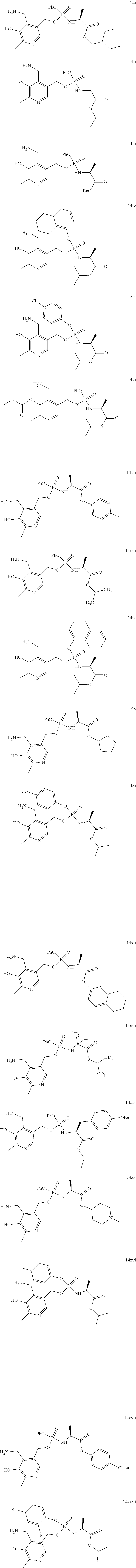
its stereoisomer, salt, hydrate, solvate, isotope or crystalline form thereof.

47. The method of claim 39, wherein the compound is 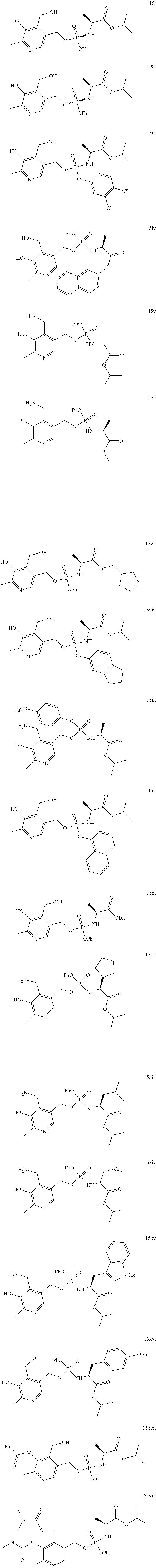 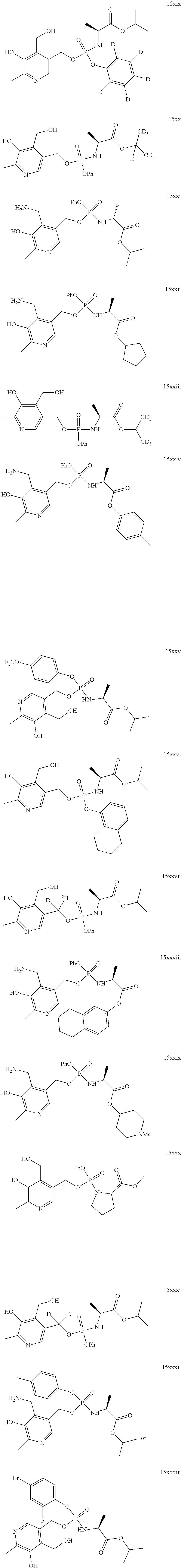
its stereoisomer, salt, hydrate, solvate, isotope or crystalline form thereof.

48. The method of claim 39, wherein the compound is [chemical structure]
its stereoisomer, salt, hydrate, solvate, isotope or crystalline form thereof.

49. The method of claim 39, wherein the compound is represented by Formula Ia 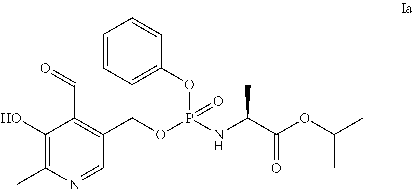 [SMILES: Cc1ncc(COP(=O)(N[C@@H](C)C(=O)OC(C)C)Oc2ccccc2)c(C=O)c1O]
its stereoisomer, salt, hydrate, solvate, isotope or crystalline form thereof.
